SEQUENCE LISTING (150 sequences)

SEQ ID NO: 1 - protein, 560 aa, Artificial Sequence
  Synthetic polypeptide
METDTLLLWVLLLWVPGSTGAGGSDYKDDDDKGGSSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 20 aa, Artificial Sequence
  Synthetic polypeptide
METDTLLLWVLLLWVPGSTG

SEQ ID NO: 3 - protein, 8 aa, Artificial Sequence
  Synthetic polypeptide
DYKDDDDK

SEQ ID NO: 4 - protein, 114 aa, Artificial Sequence
  Synthetic polypeptide
SPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 5 - protein, 229 aa, Artificial Sequence
  Synthetic polypeptide
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 28 aa, Artificial Sequence
  Synthetic polypeptide
MFWVLVVVGGVLACYSLLVTVAFIIFWV

SEQ ID NO: 7 - protein, 42 aa, Artificial Sequence
  Synthetic polypeptide
KRGRKKLLYIFKQPFMRPVQTTQEEDGCSCRFPEEEEGGCEL

SEQ ID NO: 8 - protein, 112 aa, Artificial Sequence
  Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 606 aa, Artificial Sequence
  Synthetic polypeptide
METDTLLLWVLLLWVPGSTGAGGSDYKDDDDKGGSSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 20 aa, Artificial Sequence
  Synthetic polypeptide
GGGGSGGGGSGGGGSGGGGS

SEQ ID NO: 11 - protein, 243 aa, Artificial Sequence
  Synthetic polypeptide
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIK

SEQ ID NO: 12 - protein, 12 aa, Artificial Sequence
  Synthetic polypeptide
ESKYGPPCPPCP

SEQ ID NO: 13 - protein, 560 aa, Artificial Sequence
  Synthetic polypeptide
METDTLLLWVLLLWVPGSTGAGGSDYKDDDDKGGSSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRRGRRRLLYIFRQPFMRPVQTTQEEDGCSCRFPEEEEGGCELRVRFSRSADAPAYQQGQNQLYNELNLGRREEYDVLDRRRGRDPEMGGRPRRRNPQEGLYNELQRDRMAEAYSEIGMRGERRRGRGHDGLYQGLSTATRDTYDALHMQALPPR

SEQ ID NO: 14 - protein, 42 aa, Artificial Sequence
  Synthetic polypeptide
RRGRRRLLYIFRQPFMRPVQTTQEEDGCSCRFPEEEEGGCEL

SEQ ID NO: 15 - protein, 112 aa, Artificial Sequence
  Synthetic polypeptide
RVRFSRSADAPAYQQGQNQLYNELNLGRREEYDVLDRRRGRDPEMGGRPRRRNPQEGLYNELQRDRMAEAYSEIGMRGERRRGRGHDGLYQGLSTATRDTYDALHMQALPPR

SEQ ID NO: 16 - protein, 606 aa, Artificial Sequence
  Synthetic polypeptide
METDTLLLWVLLLWVPGSTGAGGSDYKDDDDKGGSSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVRRGRRRLLYIFRQPFMRPVQTTQEEDGCSCRFPEEEEGGCELRVRFSRSADAPAYQQGQNQLYNELNLGRREEYDVLDRRRGRDPEMGGRPRRRNPQEGLYNELQRDRMAEAYSEIGMRGERRRGRGHDGLYQGLSTATRDTYDALHMQALPPR

SEQ ID NO: 17 - protein, 562 aa, Artificial Sequence
  Synthetic polypeptide
METDTLLLWVLLLWVPGSTGAGGSDYKDDDDKGGSSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 41 aa, Artificial Sequence
  Synthetic polypeptide
RSKRSRGGHSDYMNMTPRRPGPTRKHYQPYAPPRDFAAYRS

SEQ ID NO: 19 - protein, 568 aa, Artificial Sequence
  Synthetic polypeptide
METDTLLLWVLLLWVPGSTGAGGSDYKDDDDKGGSLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 122 aa, Artificial Sequence
  Synthetic polypeptide
LTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 21 - protein, 614 aa, Artificial Sequence
  Synthetic polypeptide
METDTLLLWVLLLWVPGSTGAGGSDYKDDDDKGGSLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 570 aa, Artificial Sequence
  Synthetic polypeptide
METDTLLLWVLLLWVPGSTGAGGSDYKDDDDKGGSLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 616 aa, Artificial Sequence
  Synthetic polypeptide
METDTLLLWVLLLWVPGSTGAGGSDYKDDDDKGGSLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 19 aa, Artificial Sequence
  Synthetic polypeptide
EGRGSLLTCGDVEENPGPR

SEQ ID NO: 25 - protein, 608 aa, Artificial Sequence
  Synthetic polypeptide
METDTLLLWVLLLWVPGSTGAGGSDYKDDDDKGGSSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 246 aa, Artificial Sequence
  Synthetic polypeptide
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGSTSGSGKPGSGEGSTKGDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRA

SEQ ID NO: 27 - protein, 244 aa, Artificial Sequence
  Synthetic polypeptide
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGSTSGSGKPGSGEGSTKGDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 28 - protein, 15 aa, Artificial Sequence
  Synthetic polypeptide
GGGGSGGGGSGGGGS

SEQ ID NO: 29 - protein, 120 aa, Artificial Sequence
  Synthetic polypeptide
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSS

SEQ ID NO: 30 - protein, 108 aa, Artificial Sequence
  Synthetic polypeptide
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIK

SEQ ID NO: 31 - protein, 5 aa, Artificial Sequence
  Synthetic polypeptide
SNVIS

SEQ ID NO: 32 - protein, 17 aa, Artificial Sequence
  Synthetic polypeptide
GVIPIVDIANYAQRFKG

SEQ ID NO: 33 - protein, 11 aa, Artificial Sequence
  Synthetic polypeptide
PRAFVLDAMDY

SEQ ID NO: 34 - protein, 12 aa, Artificial Sequence
  Synthetic polypeptide
RASQSLGSSYLA

SEQ ID NO: 35 - protein, 7 aa, Artificial Sequence
  Synthetic polypeptide
GASSRAP

SEQ ID NO: 36 - protein, 9 aa, Artificial Sequence
  Synthetic polypeptide
QQYADSPIT

SEQ ID NO: 37 - protein, 217 aa, Artificial Sequence
  Synthetic polypeptide
APEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 114 aa, Artificial Sequence
  Synthetic polypeptide
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSS

SEQ ID NO: 39 - protein, 112 aa, Artificial Sequence
  Synthetic polypeptide
DVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 40 - protein, 5 aa, Artificial Sequence
  Synthetic polypeptide
DYYIH

SEQ ID NO: 41 - protein, 17 aa, Artificial Sequence
  Synthetic polypeptide
RIDPENDETKYGPIFQG

SEQ ID NO: 42 - protein, 5 aa, Artificial Sequence
  Synthetic polypeptide
RGGVY

SEQ ID NO: 43 - protein, 16 aa, Artificial Sequence
  Synthetic polypeptide
KSSQSLLDSDGKTYLN

SEQ ID NO: 44 - protein, 7 aa, Artificial Sequence
  Synthetic polypeptide
LVSKLDS

SEQ ID NO: 45 - protein, 9 aa, Artificial Sequence
  Synthetic polypeptide
WQGTHFPGT

SEQ ID NO: 46 - protein, 113 aa, Artificial Sequence [sequence omitted]

SEQ ID NO: 47 - protein, 115 aa, Artificial Sequence
  Synthetic polypeptide
DVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRA

SEQ ID NO: 48 - protein, 5 aa, Artificial Sequence
  Synthetic polypeptide
GYNMN

SEQ ID NO: 49 - protein, 17 aa, Artificial Sequence
  Synthetic polypeptide
AIDPYYGGTSYNQKFKG

SEQ ID NO: 50 - protein, 4 aa, Artificial Sequence
  Synthetic polypeptide
GMEY

SEQ ID NO: 51 - protein, 16 aa, Artificial Sequence
  Synthetic polypeptide
RSSQSLVHRNGNTYLH

SEQ ID NO: 52 - protein, 7 aa, Artificial Sequence
  Synthetic polypeptide
KVSNRFS

SEQ ID NO: 53 - protein, 10 aa, Artificial Sequence
  Synthetic polypeptide
SQSTHVPPLT

SEQ ID NO: 54 - protein, 124 aa, Artificial Sequence
  Synthetic polypeptide
EVQLVESGGGLVQPGGSLRLSCAASGYAFTNYLIEWVRQAPGKGLEWVGVINPGSGGSNYNEKFKGRATISADNSKNTLYLQMNSLRAEDTAVYYCARSGGFYFDYWGQGTLVTVSSASTKGPS

SEQ ID NO: 55 - protein, 116 aa, Artificial Sequence
  Synthetic polypeptide
DIQMTQSPSSLSASVGDRVTITCRASQSVLYSSNQKNYLAWYQQKPGKAPKLLIYWASTRESGVPSRFSGSGSGTDFTLTISSLQPEDFATYYCHQYLSSDTFGQGTKVEIKRTVA

SEQ ID NO: 56 - protein, 10 aa, Artificial Sequence
  Synthetic polypeptide
GYAFTNYLIE

SEQ ID NO: 57 - protein, 17 aa, Artificial Sequence
  Synthetic polypeptide
VINPGSGGSNYNEKFKG

SEQ ID NO: 58 - protein, 8 aa, Artificial Sequence
  Synthetic polypeptide
SGGFYFDY

SEQ ID NO: 59 - protein, 17 aa, Artificial Sequence
  Synthetic polypeptide
RASQSVLYSSNQKNYLA

SEQ ID NO: 60 - protein, 7 aa, Artificial Sequence
  Synthetic polypeptide
WASTRES

SEQ ID NO: 61 - protein, 8 aa, Artificial Sequence
  Synthetic polypeptide
HQYLSSDT

SEQ ID NO: 62 - protein, 123 aa, Artificial Sequence
  Synthetic polypeptide
EVQLVESGGGVVQPGRSLRLSCAASGFTFSSYGMHWVRQAPGKELEWVAVISYDGSIKYYADSVKGRFTISRDNSKNTLYLQMNSLRAEDTAVYYCARTGEYSGYDTDPQYSWGQGTTVTVSS

SEQ ID NO: 63 - protein, 107 aa, Artificial Sequence
  Synthetic polypeptide
EIVLTQSPSSLSASVGDRVTITCRSSQGIGDDLGWYQQKPGKAPILLIYGTSTLQSGVPSRFSGSGSGTDFTLTINSLQPEDFATYYCLQDSNYPLTFGGGTRLEIK

SEQ ID NO: 64 - protein, 5 aa, Artificial Sequence
  Synthetic polypeptide
SYGMH

SEQ ID NO: 65 - protein, 17 aa, Artificial Sequence
  Synthetic polypeptide
VISYDGSIKYYADSVKG

SEQ ID NO: 66 - protein, 14 aa, Artificial Sequence
  Synthetic polypeptide
TGEYSGYDTDPQYS

SEQ ID NO: 67 - protein, 11 aa, Artificial Sequence
  Synthetic polypeptide
RSSQGIGDDLG

SEQ ID NO: 68 - protein, 7 aa, Artificial Sequence
  Synthetic polypeptide
GTSTLQS

SEQ ID NO: 69 - protein, 9 aa, Artificial Sequence
  Synthetic polypeptide
LQDSNYPLT

SEQ ID NO: 70 - protein, 5 aa, Artificial Sequence
  Synthetic polypeptide
DKTHT

SEQ ID NO: 71 - protein, 4 aa, Artificial Sequence
  Synthetic polypeptide
CPPC

SEQ ID NO: 72 - protein, 15 aa, Artificial Sequence
  Synthetic polypeptide
CPEPKSCDTPPPCPR

SEQ ID NO: 73 - protein, 12 aa, Artificial Sequence
  Synthetic polypeptide
ELKTPLGDTTHT

SEQ ID NO: 74 - protein, 10 aa, Artificial Sequence
  Synthetic polypeptide
KSCDKTHTCP

SEQ ID NO: 75 - protein, 7 aa, Artificial Sequence
  Synthetic polypeptide
KCCVDCP

SEQ ID NO: 76 - protein, 7 aa, Artificial Sequence
  Synthetic polypeptide
KYGPPCP

SEQ ID NO: 77 - protein, 15 aa, Artificial Sequence
  Synthetic polypeptide
EPKSCDKTHTCPPCP

SEQ ID NO: 78 - protein, 17 aa, Artificial Sequence
  Synthetic polypeptide
ELKTPLGDTTHTCPRCP

SEQ ID NO: 79 - protein, 12 aa, Artificial Sequence
  Synthetic polypeptide
SPNMVPHAHHAQ

SEQ ID NO: 80 - protein, 12 aa, Artificial Sequence
  Synthetic polypeptide
ESKYGPPCPPCP

SEQ ID NO: 81 - protein, 15 aa, Artificial Sequence
  Synthetic polypeptide
EPKSCDKTYTCPPCP

SEQ ID NO: 82 - protein, 45 aa, Artificial Sequence
  Synthetic polypeptide
TTTPAPRPPTPAPTIASQPLSLRPEACRPAAGGAVHTRGLDFACD

SEQ ID NO: 83 - protein, 110 aa, Artificial Sequence
  Synthetic polypeptide
APEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAK

SEQ ID NO: 84 - protein, 107 aa, Artificial Sequence
  Synthetic polypeptide
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 85 - protein, 27 aa, Artificial Sequence
  Synthetic polypeptide
FWVLVVVGGVLACYSLLVTVAFIIFWV

SEQ ID NO: 86 - protein, 23 aa, Artificial Sequence
  Synthetic polypeptide
LGLLVAGVLVLLVSLGVAIHLCC

SEQ ID NO: 87 - protein, 25 aa, Artificial Sequence
  Synthetic polypeptide
ALIVLGGVAGLLLFIGLGIFFCVRC

SEQ ID NO: 88 - protein, 23 aa, Artificial Sequence
  Synthetic polypeptide
LCYLLDGILFIYGVILTALFLRV

SEQ ID NO: 89 - protein, 26 aa, Artificial Sequence
  Synthetic polypeptide
WVLVVVGGVLACYSLLVTVAFIIFWV

SEQ ID NO: 90 - protein, 26 aa, Artificial Sequence
  Synthetic polypeptide
VAAILGLGLVLGLLGPLAILLALYLL

SEQ ID NO: 91 - protein, 24 aa, Artificial Sequence
  Synthetic polypeptide
ALPAALAVISFLLGLGLGVACVLA

SEQ ID NO: 92 - protein, 113 aa, Artificial Sequence
  Synthetic polypeptide
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 93 - protein, 112 aa, Artificial Sequence
  Synthetic polypeptide
MGGLEPCSRLLLLPLLLAVSGLRPVQAQAQSDCSCSTVSPGVLAGIVMGDLVLTVLIALAVYFLGRLVPRGRGAAEATRKQRITETESPYQELQGQRSDVYSDLNTQRPYYK

SEQ ID NO: 94 - protein, 102 aa, Artificial Sequence
  Synthetic polypeptide
MGGLEPCSRLLLLPLLLAVSDCSCSTVSPGVLAGIVMGDLVLTVLIALAVYFLGRLVPRGRGAAEAATRKQRITETESPYQELQGQRSDVYSDLNTQRPYYK

SEQ ID NO: 95 - protein, 101 aa, Artificial Sequence
  Synthetic polypeptide
MGGLEPCSRLLLLPLLLAVSDCSCSTVSPGVLAGIVMGDLVLTVLIALAVYFLGRLVPRGRGAAEATRKQRITETESPYQELQGQRSDVYSDLNTQRPYYK

SEQ ID NO: 96 - protein, 86 aa, Artificial Sequence
  Synthetic polypeptide
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 97 - protein, 21 aa, Artificial Sequence
  Synthetic polypeptide
DGVYTGLSTRNQETYETLKHE

SEQ ID NO: 98 - protein, 171 aa, Artificial Sequence
  Synthetic polypeptide
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 99 - protein, 127 aa, Artificial Sequence
  Synthetic polypeptide
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 100 - protein, 21 aa, Artificial Sequence
  Synthetic polypeptide
DQVYQPLRDRDDAQYSHLGGN

SEQ ID NO: 101 - protein, 207 aa, Artificial Sequence
  Synthetic polypeptide
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 102 - protein, 21 aa, Artificial Sequence
  Synthetic polypeptide
NPDYEPIRKGQRDLYSGLNQR

SEQ ID NO: 103 - protein, 182 aa, Artificial Sequence
  Synthetic polypeptide
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 104 - protein, 21 aa, Artificial Sequence
  Synthetic polypeptide
DQLYQPLKDREDDQYSHLQGN

SEQ ID NO: 105 - protein, 163 aa, Artificial Sequence
  Synthetic polypeptide
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 164 aa, Artificial Sequence
  Synthetic polypeptide
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 107 - protein, 21 aa, Artificial Sequence
  Synthetic polypeptide
NQLYNELNLGRREEYDVLDKR

SEQ ID NO: 108 - protein, 22 aa, Artificial Sequence
  Synthetic polypeptide
EGLYNELQKDKMAEAYSEIGMK

SEQ ID NO: 109 - protein, 21 aa, Artificial Sequence
  Synthetic polypeptide
DGLYQGLSTATKDTYDALHMQ

SEQ ID NO: 110 - protein, 226 aa, Artificial Sequence
  Synthetic polypeptide
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 111 - protein, 188 aa, Artificial Sequence
  Synthetic polypeptide
MPGGPGVLQALPATIFLLFLLSAVYLGPGCQALWMHKVPASLMVSLGEDAHFQCPHNSSNNANVTWWRVLHGNYTWPPEFLGPGEDPNEPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 112 - protein, 21 aa, Artificial Sequence
  Synthetic polypeptide
ENLYEGLNLDDCSMYEDISRG

SEQ ID NO: 113 - protein, 68 aa, Artificial Sequence
  Synthetic polypeptide
FWVLVVVGGVLACYSLLVTVAFIIFWVRSKRSRLLHSDYMNMTPRRPGPTRKHYQPYAPPRDFAAYRS

SEQ ID NO: 114 - protein, 44 aa, Artificial Sequence
  Synthetic polypeptide
FWVRSKRSRLLHSDYMNMTPRRPGPTRKHYQPYAPPRDFAAYRS

SEQ ID NO: 115 - protein, 35 aa, Artificial Sequence
  Synthetic polypeptide
TKKKYSSSVHDPNGEYMFMRAVNTAKKSRLTDVTL

SEQ ID NO: 116 - protein, 37 aa, Artificial Sequence
  Synthetic polypeptide
RRDQRLPPDAHKPPGGGSFRTPIQEEQADAHSTLAKI

SEQ ID NO: 117 - protein, 114 aa, Artificial Sequence
  Synthetic polypeptide
CCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 118 - protein, 49 aa, Artificial Sequence
  Synthetic polypeptide
HQRRKYRSNKGESPVEPAEPCRYSCPREEEGSTIPIQEDYRKPEPACSP

SEQ ID NO: 119 - protein, 187 aa, Artificial Sequence
  Synthetic polypeptide
RRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 120 - protein, 54 aa, Artificial Sequence
  Synthetic polypeptide
HIWQLRSQCMWPRETQLLLEVPPSTEDARSCQFPEEERGERSAEEKGRLGDLWV

SEQ ID NO: 121 - protein, 60 aa, Artificial Sequence
  Synthetic polypeptide
CVKRRKPRGDVVKVIVSVQRKRQEAEGEATVIEALQAPPDVTTVAVEETIPSFTGRSPNH

SEQ ID NO: 122 - protein, 9 aa, Artificial Sequence
  Synthetic polypeptide
YPYDVPDYA

SEQ ID NO: 123 - protein, 10 aa, Artificial Sequence
  Synthetic polypeptide
EQKLISEEDL

SEQ ID NO: 124 - protein, 5 aa, Artificial Sequence
  Synthetic polypeptide
GSSSG

SEQ ID NO: 125 - protein, 4 aa, Artificial Sequence
  Synthetic polypeptide
GGSG

SEQ ID NO: 126 - protein, 5 aa, Artificial Sequence
  Synthetic polypeptide
GGSGG

SEQ ID NO: 127 - protein, 5 aa, Artificial Sequence
  Synthetic polypeptide
GSGSG

SEQ ID NO: 128 - protein, 5 aa, Artificial Sequence
  Synthetic polypeptide
GSGGG

SEQ ID NO: 129 - protein, 5 aa, Artificial Sequence
  Synthetic polypeptide
GGGSG

SEQ ID NO: 130 - protein, 5 aa, Artificial Sequence
  Synthetic polypeptide
EAAAK

SEQ ID NO: 131 - protein, 792 aa, Artificial Sequence
  Synthetic polypeptide
METDTLLLWVLLLWVPGSTGAGGSDYKDDDDKGGSSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMGRGLLRGLWPLHIVLWTRIASTIPPYPYDVPDYAHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 132 - protein, 568 aa, Artificial Sequence
  Synthetic polypeptide
METDTLLLWVLLLWVPGSTGAGGSDYKDDDDKGGSLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRRGRRRLLYIFRQPFMRPVQTTQEEDGCSCRFPEEEEGGCELRVRFSRSADAPAYQQGQNQLYNELNLGRREEYDVLDRRRGRDPEMGGRPRRRNPQEGLYNELQRDRMAEAYSEIGMRGERRRGRGHDGLYQGLSTATRDTYDALHMQALPPR

SEQ ID NO: 133 - protein, 614 aa, Artificial Sequence
  Synthetic polypeptide
METDTLLLWVLLLWVPGSTGAGGSDYKDDDDKGGSLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVRRGRRRLLYIFRQPFMRPVQTTQEEDGCSCRFPEEEEGGCELRVRFSRSADAPAYQQGQNQLYNELNLGRREEYDVLDRRRGRDPEMGGRPRRRNPQEGLYNELQRDRMAEAYSEIGMRGERRRGRGHDGLYQGLSTATRDTYDALHMQALPPR

SEQ ID NO: 134 - protein, 800 aa, Artificial Sequence
  Synthetic polypeptide
METDTLLLWVLLLWVPGSTGAGGSDYKDDDDKGGSLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMGRGLLRGLWPLHIVLWTRIASTIPPYPYDVPDYAHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 135 - protein, 794 aa, Artificial Sequence
  Synthetic polypeptide
METDTLLLWVLLLWVPGSTGAGGSDYKDDDDKGGSSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMGRGLLRGLWPLHIVLWTRIASTIPPYPYDVPDYAHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 136 - protein, 545 aa, Artificial Sequence
  Synthetic polypeptide
METDTLLLWVLLLWVPGSTGSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 591 aa, Artificial Sequence
  Synthetic polypeptide
METDTLLLWVLLLWVPGSTGSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 545 aa, Artificial Sequence
  Synthetic polypeptide
METDTLLLWVLLLWVPGSTGSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRRGRRRLLYIFRQPFMRPVQTTQEEDGCSCRFPEEEEGGCELRVRFSRSADAPAYQQGQNQLYNELNLGRREEYDVLDRRRGRDPEMGGRPRRRNPQEGLYNELQRDRMAEAYSEIGMRGERRRGRGHDGLYQGLSTATRDTYDALHMQALPPR

SEQ ID NO: 139 - protein, 591 aa, Artificial Sequence
  Synthetic polypeptide
METDTLLLWVLLLWVPGSTGSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVRRGRRRLLYIFRQPFMRPVQTTQEEDGCSCRFPEEEEGGCELRVRFSRSADAPAYQQGQNQLYNELNLGRREEYDVLDRRRGRDPEMGGRPRRRNPQEGLYNELQRDRMAEAYSEIGMRGERRRGRGHDGLYQGLSTATRDTYDALHMQALPPR

SEQ ID NO: 140 - protein, 547 aa, Artificial Sequence
  Synthetic polypeptide
METDTLLLWVLLLWVPGSTGSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 593 aa, Artificial Sequence
  Synthetic polypeptide
METDTLLLWVLLLWVPGSTGSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - protein, 553 aa, Artificial Sequence
  Synthetic polypeptide
METDTLLLWVLLLWVPGSTGLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - protein, 599 aa, Artificial Sequence
  Synthetic polypeptide
METDTLLLWVLLLWVPGSTGLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 144 - protein, 555 aa, Artificial Sequence
  Synthetic polypeptide
METDTLLLWVLLLWVPGSTGLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 145 - protein, 601 aa, Artificial Sequence
  Synthetic polypeptide
METDTLLLWVLLLWVPGSTGLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 146 - protein, 553 aa, Artificial Sequence
  Synthetic polypeptide
METDTLLLWVLLLWVPGSTGLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRRGRRRLLYIFRQPFMRPVQTTQEEDGCSCRFPEEEEGGCELRVRFSRSADAPAYQQGQNQLYNELNLGRREEYDVLDRRRGRDPEMGGRPRRRNPQEGLYNELQRDRMAEAYSEIGMRGERRRGRGHDGLYQGLSTATRDTYDALHMQALPPR

SEQ ID NO: 147 - protein, 599 aa, Artificial Sequence
  Synthetic polypeptide
METDTLLLWVLLLWVPGSTGLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVRRGRRRLLYIFRQPFMRPVQTTQEEDGCSCRFPEEEEGGCELRVRFSRSADAPAYQQGQNQLYNELNLGRREEYDVLDRRRGRDPEMGGRPRRRNPQEGLYNELQRDRMAEAYSEIGMRGERRRGRGHDGLYQGLSTATRDTYDALHMQALPPR

SEQ ID NO: 148 - protein, 5 aa, Artificial Sequence
  Synthetic polypeptide
GSGGS

SEQ ID NO: 149 - protein, 4 aa, Artificial Sequence
  Synthetic polypeptide
GGGS

SEQ ID NO: 150 - protein, 5 aa, Artificial Sequence
  Synthetic polypeptide
GGGGS

(19) United States
(12) Patent Application Publication
(10) Pub. No.: US 2023/0322894 A1
(43) Pub. Date: Oct. 12, 2023

(54) METHODS AND COMPOSITIONS FOR TREATING GLIOBLASTOMA

(71) Applicant: THE REGENTS OF THE UNIVERSITY OF CALIFORNIA, Oakland, CA (US)

(72) Inventors: Yvonne Yu-Hsuan CHEN, Los Angeles, CA (US); Andrew J. HOU, Los Angeles, CA (US)

(73) Assignee: THE REGENTS OF THE UNIVERSITY OF CALIFORNIA, Oakland, CA (US)

(21) Appl. No.: 18/042,985

(22) PCT Filed: Aug. 25, 2021
(86) PCT No.: PCT/US2021/047604

§ 371 Date: Feb. 24, 2023

Related U.S. Application Data
(60) Provisional application No. 63/070,550, filed on Aug. 26, 2020.
(60) Provisional application No. 63/171,187, filed on Apr. 6, 2021.
(60) Provisional application No. 63/218,761, filed on Jul. 6, 2021.

(51) Int. Cl. C07K 14/725; C07K 14/54; A61P 35/00

(52) U.S. Cl. CPC C07K 14/7051; C07K 14/5437; A61P 35/00; C07K 2319/02; C07K 2319/03; C07K 2319/33; C07K 2319/43

(57) ABSTRACT

The current disclosure provides for novel multi-specific CAR molecules for the treatment of glioblastoma (also called GBM or glioblastoma multiforme). This disclosure also describes nucleic acids encoding for the polypeptides, expression vectors comprising the nucleic acids, cells and/or populations of cells expressing the polypeptides and/or comprising the nucleic acids or expression vectors of the disclosure, and compositions comprising the polypeptides, nucleic acids, or cells.

This application claims the benefit of priority of U.S. Provisional Application No. 63/070,550, filed Aug. 26, 2020, U.S. Provisional Application No. 63/171,187, filed Apr. 6, 2021, and U.S. Provisional Application No. 63/218,761, filed Jul. 6, 2021, each of which are hereby incorporated by reference in their entirety.

This invention was made with government support under Grant No. CA211015, awarded by National Institutes of Health. The government has certain rights in the invention.

### BACKGROUND

### II. Field of the Invention

This invention relates generally to the fields of molecular biology and immunotherapy.

### III. Background

Glioblastoma multiforme (GBM) is the most common type of primary brain tumors in adults, and the median survival period has remained at 12-16 months from the time of diagnosis over the past few decades. Conventional therapies such as surgery, chemotherapy, and radiation almost invariably fail to eradicate tumor, resulting in relapse within weeks or months. Consequently, GBM has been an active area of research for new treatment options such as adoptive T-cell therapy. Two major challenges have limited the efficacy of T-cell therapy for GBM thus far. First, the GBM tumor microenvironment is strongly immunosuppressive, characterized by a high level of transforming growth factor beta (TGF-β), which simultaneously promote tumor growth and potently suppress the function of T cells. Second, GBM tumors are highly heterogeneous in antigen expression, thus T cells engineered to target a single antigen are generally unable to recognize and eradicate all tumor cells present. Therefore, there is a need in the art for improved GBM therapies.

### SUMMARY OF THE DISCLOSURE

The current disclosure provides a need in the art by providing for novel multi-specific CAR molecules for the treatment of glioblastoma (also called GBM or glioblastoma multiforme). Accordingly, aspects of the disclosure relate to a polypeptide comprising a multi-specific chimeric antigen receptor comprising a IL13 polypeptide of SEQ ID NO:4 or 20, a glioblastoma antigen binding region, a peptide spacer, a transmembrane domain, and a cytoplasmic region comprising a co-stimulatory region and a primary intracellular signaling domain; wherein the glioblastoma antigen binding region comprises a GD2 or EGFRvIII binding region.

A further aspect relates to a polypeptide comprising a multi-specific chimeric antigen receptor (CAR) comprising an IL13 polypeptide of SEQ ID NO:4 or 20, a TGF-β binding region, a peptide spacer, a transmembrane domain, and a cytoplasmic region comprising a co-stimulatory region and a primary intracellular signaling domain.

Yet further aspects relate to a polypeptide comprising a multi-specific chimeric antigen receptor (CAR) comprising a glioblastoma antigen binding region, a TGF-β binding region, a peptide spacer, a transmembrane domain, and a cytoplasmic region comprising a co-stimulatory region and a primary intracellular signaling domain; wherein the glioblastoma antigen binding region comprises an anti-GD2 scFv having a variable heavy (VH) and variable light (VL) region, wherein the VH region comprises SEQ ID NO:48 (HCDR1), SEQ ID NO:49 (HCDR2); and SEQ ID NO:50 (HCDR3) and the VL region comprises SEQ ID NO:51 (LCDR1), SEQ ID NO:52 (LCDR2); and SEQ ID NO:53 (LCDR3). Further aspects relate to a polypeptide comprising a multi-specific chimeric antigen receptor (CAR) comprising a glioblastoma antigen binding region, a TGF-β binding region, a peptide spacer, a transmembrane domain, and a cytoplasmic region comprising a co-stimulatory region and a primary intracellular signaling domain; wherein the glioblastoma antigen binding region comprises an anti-GD2 scFv having a variable heavy (VH) and variable light (VL) region, wherein the VH region comprises the HCDR1, HCDR2; and HCDR3 from the VH of SEQ ID NO:46 and the VL region comprises LCDR1, LCDR2; and LCDR3 from the VL of SEQ ID NO:47.

Also provided is a polypeptide comprising a multi-specific chimeric antigen receptor (CAR) comprising a glioblastoma antigen binding region, a TGF-β binding region, a peptide spacer, a transmembrane domain, and a cytoplasmic region comprising a co-stimulatory region and a primary intracellular signaling domain; wherein the glioblastoma antigen binding region comprises an EGFRvIII binding region.

This disclosure also describes nucleic acids encoding for the polypeptides of the disclosure, expression vectors comprising the nucleic acids of the disclosure, cells and/or populations of cells expressing the polypeptides of the disclosure and/or comprising the nucleic acids or expression vectors of the disclosure, and compositions comprising the polypeptides, nucleic acids, or cells of the disclosure. The compositions may be in the form of a pharmaceutically acceptable formulation.

Aspects of the disclosure also relate to a method of making a cell that expresses a polypeptide comprising introducing into a cell a nucleic acid of the disclosure. Further method aspects relate to a method for stimulating an immune response or for treating glioblastoma in a subject, the method comprising administering to the subject an effective amount of a composition, cell, or polypeptide of the disclosure. Also provided is a method for expanding therapeutic T cells in vitro, the method comprising contacting an in vitro T cell of the disclosure with a composition comprising TGF-β.

Aspects of the disclosure relate to polypeptides comprising one or more glioblastoma antigen binding regions. In some aspects, the glioblastoma antigen binding region comprises a GD2 binding region. The structure of GD2 is known in the art. GD2 is a disialoganglioside belonging to b-series ganglioside. It comprises five monosaccharides linked to ceramide, with the carbohydrate sequence of GalNAcβ1-4(NeuAcα2-8NeuAcα2-3)Galβ1-4Glcβ1-1. GD2 binding regions, such as anti-GD2 antibody binding regions are known in the art. In some aspects, the GD2 binding region comprises an anti-GD2 scFv having a variable heavy (VH) and variable light (VL) region, wherein the VH region comprises SEQ ID NO:48 (HCDR1), SEQ ID NO:49 (HCDR2); and SEQ ID NO:50 (HCDR3) and the VL region comprises SEQ ID NO:51 (LCDR1), SEQ ID NO:52 (LCDR2); and SEQ ID NO:53 (LCDR3).

In some aspects, the glioblastoma antigen binding region comprises a EGFRvIII antigen binding region. EGFRvIII is a variant of EGFR that lacks amino acids 6-273, and deletion of those 268 amino acids creates a junction site with a new glycine residue between amino acids 5 and 274. In some aspects, the EGFRvIII binding region comprises an anti-EGFRvIII scFv having a variable heavy (VH) and variable light (VL) region, wherein the VH region comprises SEQ ID NO:40 (HCDR1), SEQ ID NO:41 (HCDR2); and SEQ ID NO:42 (HCDR3) and the VL region comprises SEQ ID NO:43 (LCDR1), SEQ ID NO:44 (LCDR2); and SEQ ID NO:45 (LCDR3).

In some aspects, the polypeptides comprise a TGF-β binding region. In some aspects, the TGF-β binding region comprises a scFv having a variable heavy (VH) and variable light (VL) region, wherein the VH region comprises SEQ ID NO:31 (HCDR1), SEQ ID NO:32 (HCDR2); and SEQ ID NO:33 (HCDR3) and the VL region comprises SEQ ID NO:34 (LCDR1), SEQ ID NO:35 (LCDR2); and SEQ ID NO:36 (LCDR3).

In some aspects, LCDR1 of a GD2, EGRvIII, or TGF-β binding region comprises an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:51, 43, or 34, respectively. In some aspects, LCDR2 of a GD2, EGRvIII, or TGF-β binding region comprises an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:52, 44, or 35, respectively. In some aspects, LCDR3 of a GD2, EGRvIII, or TGF-β binding region comprises an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:53, 45, or 36, respectively. In some aspects, HCDR1 of a GD2, EGRvIII, or TGF-β binding region comprises an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:48, 40, or 31, respectively. In some aspects, HCDR2 of a GD2, EGRvIII, or TGF-β binding region comprises an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:49, 41, or 32, respectively. In some aspects, HCDR3 of a GD2, EGRvIII, or TGF-β binding region comprises an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:50, 42, or 33, respectively.

In some aspects, the GD2 binding region comprises a VH with an amino acid sequence having at least 80% sequence identity to SEQ ID NO:46 and/or a VL with an amino acid sequence having at least 80% sequence identity to SEQ ID NO:47. In some aspects, the GD2 binding region comprises a VH with the amino acid sequence of SEQ ID NO:46 and/or a VL with the amino acid sequence of SEQ ID NO:47. In some aspects, the GD2 binding region comprises a VH with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:46 and/or a VL with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:47. The GD2 binding region may also be one that comprises an anti-GD2 scFv having an amino acid sequence with at least 80% sequence identity to SEQ ID NO:26. In some aspects, the GD2 binding region comprises an anti-GD2 scFv having the amino acid sequence of SEQ ID NO:26. Other aspects include GD2 binding regions that comprise an anti-GD2 scFv having an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:26. In some aspects, the GD2 binding region comprises a binding region that binds to membrane-bound GD2 antigen. In some aspects, the GD2 binding region comprises a binding region that binds to soluble GD2 antigen. In some aspects, the GD2 binding region comprises a binding region that binds to membrane-bound and soluble GD2 antigen.

In some aspects, the polypeptides comprise a TGF-β binding region. In some aspects, the TGF-β binding region comprises a scFv having a variable heavy (VH) and variable light (VL) region, wherein the VH region comprises SEQ ID NO:56 (HCDR1), SEQ ID NO:57 (HCDR2); and SEQ ID NO:58 (HCDR3) and the VL region comprises SEQ ID NO:59 (LCDR1), SEQ ID NO:60 (LCDR2); and SEQ ID NO:61 (LCDR3). In some aspects, the polypeptides comprise a TGF-β binding region. In some aspects, the TGF-β binding region comprises a scFv having a variable heavy (VH) and variable light (VL) region, wherein the VH region comprises SEQ ID NO:64 (HCDR1), SEQ ID NO:65 (HCDR2); and SEQ ID NO:66 (HCDR3) and the VL region comprises SEQ ID NO:67 (LCDR1), SEQ ID NO:68 (LCDR2); and SEQ ID NO:69 (LCDR3).

In some aspects, LCDR1 of a TGF-β binding region comprises an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:59 or 67. In some aspects, LCDR2 of a TGF-β binding region comprises an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:60 or 68. In some aspects, LCDR3 of a TGF-β binding region comprises an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:61 or 69. In some aspects, HCDR1 of a TGF-β binding region comprises an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:56 or 64. In some aspects, HCDR2 of a TGF-β binding region comprises an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:57 or 65. In some aspects, HCDR3 of a TGF-β binding region comprises an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:58 or 66. In some aspects, the TGF-β binding region comprises a VH with an amino acid sequence having at least 80% sequence identity to SEQ ID NO:54 and/or a VL with an amino acid sequence having at least 80% sequence identity to SEQ ID NO:55. In some aspects, the TGF-β binding region comprises a VH with the amino acid sequence of SEQ ID NO:54 and/or a VL with the amino acid sequence of SEQ ID NO:55. In some aspects, the TGF-β binding region comprises a VH with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:54 and/or a VL with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:55. In some aspects, the TGF-β binding region comprises a VH with an amino acid sequence having at least 80% sequence identity to SEQ ID NO:62 and/or a VL with an amino acid sequence having at least 80% sequence identity to SEQ ID NO:63. In some aspects, the TGF-β binding region comprises a VH with the amino acid sequence of SEQ ID NO:62 and/or a VL with the amino acid sequence of SEQ ID NO:63. In some aspects, the TGF-β binding region comprises a VH with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:62 and/or a VL with an amino acid sequence having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:63.

In some aspects, LCDR1 of a TGF-β binding region comprises an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:34. In some aspects, LCDR2 of a TGF-β binding region comprises an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:35. In some aspects, LCDR3 of a TGF-β binding region comprises an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:36. In some aspects, HCDR1 of a TGF-β binding region comprises an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:31. In some aspects, HCDR2 of a TGF-β binding region comprises an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:32. In some aspects, HCDR3 of a TGF-β binding region comprises an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:33. In some aspects, the TGF-β binding region comprises a VH with an amino acid sequence having at least 80% sequence identity to SEQ ID NO:29 and/or a VL with an amino acid sequence having at least 80% sequence identity to SEQ ID NO:30. In some aspects, the TGF-β binding region comprises a VH with the amino acid sequence of SEQ ID NO:29 and/or a VL with the amino acid sequence of SEQ ID NO:30. In some aspects, the TGF-β binding region comprises a VH with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:29 and/or a VL with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:30.

In some aspects, the EGFRvIII binding region comprises a VH with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:38 and/or a VL with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:39. In some aspects, the EGFRvIII binding region comprises a VH with an amino acid sequence having at least 80% sequence identity to SEQ ID NO:38 and/or a VL with an amino acid sequence having at least 80% sequence identity to SEQ ID NO:39. In some aspects, the EGFRvIII binding region comprises a VH with the amino acid sequence of SEQ ID NO:38 and/or a VL with the amino acid sequence of SEQ ID NO:39. In some aspects, the EGFRvIII binding region comprises a VH with the amino acid sequence of SEQ ID NO:38 and/or a VL with the amino acid sequence of SEQ ID NO:39. In some aspects, the EGFRvIII binding region comprises an anti-EGFRvIII scFv having an amino acid sequence with at least 80% sequence identity to SEQ ID NO:27. In some aspects, the EGFRvIII binding region comprises an anti-EGFRvIII scFv having an amino acid sequence with or with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:27. In some aspects, the EGFRvIII binding region comprises an anti-EGFRvIII scFv having the amino acid sequence of SEQ ID NO:27. In some aspects, the EGFRvIII binding region comprises a binding region that binds to membrane-bound EGFRvIII antigen. In some aspects, the EGFRvIII binding region comprises a binding region that binds to soluble EGFRvIII antigen. In some aspects, the EGFRvIII binding region comprises a binding region that binds to membrane-bound and soluble EGFRvIII antigen.

Aspects of the disclosure include polypeptides and CARs comprising a TGF-β binding region. In some aspects, the TGF-β binding region comprises a VH with an amino acid sequence having at least 80% sequence identity to SEQ ID NO:29 and/or a VL with an amino acid sequence having at least 80% sequence identity to SEQ ID NO:30. In some aspects, the TGF-β binding region comprises a VH with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:29 and/or a VL with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:30. In some aspects, the TGF-β binding region comprises a VH with the amino acid sequence of SEQ ID NO:29 and/or a VL with the amino acid sequence of SEQ ID NO:30. In some aspects, the TGF-β binding region comprises an anti-TGF-β scFv having an amino acid sequence with at least 80% sequence identity to SEQ ID NO:11. In some aspects, the TGF-β binding region comprises an anti-TGF-β scFv having an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:11. In some aspects, the TGF-β binding region comprises an anti-TGF-β scFv having the amino acid sequence of SEQ ID NO:11.

The GD2 binding region may comprise an anti-GD2 scFv having a variable heavy (VH) and variable light (VL) region, wherein the VH region comprises the HCDR1, HCDR2; and HCDR3 from the VH of SEQ ID NO:46 and the VL region comprises LCDR1, LCDR2; and LCDR3 from the VL of SEQ ID NO:47. The EGFRvIII binding region may comprise an anti-EGFRvIII scFv having a variable heavy (VH) and variable light (VL) region, wherein the VH region comprises the HCDR1, HCDR2; and HCDR3 from the VH of SEQ ID NO:38 and the VL region comprises LCDR1, LCDR2; and LCDR3 from the VL of SEQ ID NO:39. The TGF-β binding region may comprise a scFv having a variable heavy (VH) and variable light (VL) region, wherein the VH region comprises the HCDR1, HCDR2; and HCDR3 from the VH of SEQ ID NO:29 and the VL region comprises LCDR1, LCDR2; and LCDR3 from the VL of SEQ ID NO:30. The TGF-β binding region may comprise a scFv having a variable heavy (VH) and variable light (VL) region, wherein the VH region comprises the HCDR1, HCDR2; and HCDR3 from the VH of SEQ ID NO:54 and the VL region comprises LCDR1, LCDR2; and LCDR3 from the VL of SEQ ID NO:55. The TGF-β binding region may comprise a scFv having a variable heavy (VH) and variable light (VL) region, wherein the VH region comprises the HCDR1, HCDR2; and HCDR3 from the VH of SEQ ID NO:62 and the VL region comprises LCDR1, LCDR2; and LCDR3 from the VL of SEQ ID NO:63. In some aspects, the HCDR1, HCDR2, HCDR3, LCDR1, LCDR2, and LCDR3 is determined by the Kabat method. In some aspects, the HCDR1, HCDR2, HCDR3, LCDR1, LCDR2, and LCDR3 is determined by the IMGT method. In some aspects, the HCDR1, HCDR2, HCDR3, LCDR1, LCDR2, and LCDR3 is determined by the Chothia method. In some aspects, the HCDR1, HCDR2, HCDR3, LCDR1, LCDR2, and LCDR3 is determined by the paratome method.

“Single-chain Fv” or “scFv” antibody fragments comprise at least a portion of the VH and VL domains of an antibody, such as the CDRs of each, wherein these domains are present in a single polypeptide chain. It is contemplated that an scFv includes a CDR1, CDR2, and/or CDR3 of a heavy chain variable region and a CDR1, CDR2, and/or CDR3 of a light chain variable region in some aspects. It is further contemplated that a CDR1, CDR2, or CDR3 may comprise or consist of a sequence set forth in a SEQ ID NO provided herein as CDR1, CDR2, or CDR3, respectively. A CDR may also comprise 1, 2, 3, 4, 5, 6, 7, 8, 9, 10, 11, 12, 13, 14, 15, 16, 17, 18, 19, 20, 21, 22, 23, or more contiguous amino acid residues (or any range derivable therein) flanking one or both sides of a particular CDR sequence; therefore, there may be one or more additional amino acids at the N-terminal or C-terminal end of a particular CDR sequence, such as those shown in SEQ ID NOS:31-36, 40-45, 48-53, 56-61, and 64-69.

Aspects of the disclosure also relate to multi-specific polypeptides comprising an IL13 polypeptide and a glioblastoma antigen binding region. In some aspects, the polypeptide comprises a chimeric antigen receptor (CAR), wherein the CAR comprises in order from amino-proximal end to carboxy-proximal end: an IL13 polypeptide, a glioblastoma antigen binding region, a peptide spacer, a transmembrane domain, and a cytoplasmic region comprising a co-stimulatory region and a primary intracellular signaling domain.

It is contemplated that the IL13 polypeptide may be amino proximal to the glioblastoma antigen binding region. In other aspects, the IL13 polypeptide may be carboxy proximal to the glioblastoma antigen binding region. The TGF-β binding region may be amino proximal to the glioblastoma antigen binding region or the TGF-β binding region may be carboxy proximal to the glioblastoma antigen binding region. The IL13 polypeptide may be amino proximal to the TGF-β binding region or the IL13 polypeptide may be carboxy proximal to the TGF-β binding region. It is contemplated that the TGF-β binding region may be adjacent to the IL13 polypeptide, meaning that there are no intervening binding regions, although any two binding regions that are adjacent may be separated by a linker region. The IL13 polypeptide may be adjacent to the glioblastoma antigen binding region, or the glioblastoma antigen binding region may be adjacent to the TGF-β binding region.

The polypeptide may comprise or further comprise one or more linkers separating regions. For example, the polypeptide may comprise a linker between two binding regions, such as a linker between the IL13 polypeptide and the glioblastoma antigen binding region. The polypeptide may comprise a linker between the TGF-β binding region and the glioblastoma antigen binding region, and/or between the IL13 polypeptide and the TGF-β binding region. In certain aspects, the polypeptide comprises a tri-specific CAR comprising TGF-β binding region. The tri-specific CAR may comprise a TGF-β binding region, an IL13 polypeptide, and a glioblastoma antigen binding region.

In some aspects, the IL13Rα binding region is a IL13Rα2 binding region. In some aspects, the IL13 polypeptide excludes an IL13 polypeptide consisting of amino acids 3-114 of SEQ ID NO:4. In some aspects, the IL13 polypeptide excludes an IL13 polypeptide consisting of amino acids 11-122 of SEQ ID NO:4. In some aspects, the IL13 polypeptide comprises the C-terminal 112 amino acids of SEQ ID NO:4 or 20 and at least one additional amino acid at the N terminus. In some aspects, the IL13 polypeptide comprises the C-terminal 112 amino acids of SEQ ID NO:4 or 20 and at least, at most, or exactly 1, 2, 3, 4, 5, 6, 7, 8, 9, or 10 additional amino acids at the N-terminus. Additionally or alternatively, in some aspects, the IL13 polypeptide comprises the C-terminal 112 amino acids of SEQ ID NO:4 or 20 and at least, at most, or exactly 1, 2, 3, 4, 5, 6, 7, 8, 9, or 10 additional amino acids at the C-terminus.

In further aspects, a CAR molecule also comprises a tag that can be used to sort and/or identify the CAR molecule in a host cell. In some aspects, the tag is further defined as a therapeutic control. In some aspects, the tag or therapeutic control is less than a full-length polypeptide and is truncated. For instance, to remove one or more functional domains from the tag. In certain aspects, the truncated protein is EGFR (EGFRt), which can be used to detect expression of the CAR. In another aspect, the truncated protein is truncated low-affinity nerve growth factor receptor or (dNGFR). In other aspects, the tag is colorimetric or fluorescent. In some aspects, the tag may be separated from the CAR by a cleavage site.

In some aspects, the VH is amino proximal to the VL. In some aspects, the VH is carboxy proximal to the VL. A first region is carboxy proximal to a second region when the first region is attached to the carboxy terminus of the second region. There may be further intervening amino acid residues between the first and second regions. Thus, the regions need not be immediately adjacent, unless specifically specified as not having intervening amino acid residues. The term “amino-proximal” is similarly defined in that a first region is amino-proximal to a second region when the first region is attached to the amino terminus of the second region. Similarly, there may be further intervening amino acid residues between the first and second regions unless stated otherwise.

In a particular aspect, the CAR comprises in order from amino-proximal end to carboxy-proximal end: an IL13 polypeptide, a glioblastoma antigen binding region, a TGF-β binding region, a peptide spacer, a transmembrane domain, and a cytoplasmic region comprising a co-stimulatory region and a primary intracellular signaling domain.

The linker between two regions of the polypeptide, such as between two binding regions or between a VH and VL of the same binding region, may be a linker that comprises glycine and serine amino acids. In some aspects, the linker comprises or consists of a polypeptide with the amino acid sequence of SEQ ID NO:10 or 28. In some aspects, the linker is 4-40 amino acids in length. In some aspects, the linker is, is at least, is at most, or is about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) amino acid residues in length. In some aspects, the linker comprises at least 4 glycine and/or serine residues. In some aspects, the linker comprises at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) glycine and/or serine residues. In some aspects, the linker comprises (GGGGS)ₙ, wherein n is 1, 2, 3, 4, 5, 6, 7, 8, 9, or 10 (or any derivable range therein). In some aspects, the linker comprises, or consists of, the amino acid sequence: (EAAAK)ₙ, wherein n is 1, 2, 3, 4, 5, 6, 7, 8, 9, or 10 (or any derivable range therein).

The polypeptide may further comprise a second chimeric antigen receptor (CAR) comprising at least one antigen binding region, a second peptide spacer, a second transmembrane domain, and a second cytoplasmic region comprising a second co-stimulatory region and a second primary intracellular signaling domain. The second CAR may be a mono-specific or multi-specific CAR, such as a bi-specific or tri-specific CAR. In certain aspects, the second CAR comprises an antigen binding region to TGF-β. The first CAR and the second CAR may be separated by one or more peptide cleavage site(s). The peptide cleavage site may be a peptide cleavage site known in the art, such as a Furin cleavage site or a 2A cleavage site. The 2A cleavage site may comprise one or more of a P2A, F2A, E2A, or T2A cleavage site. In some aspects, the peptide cleavage site comprises a T2A cleavage site. The T2A cleavage site may comprise an amino acid sequence of SEQ ID NO:24. Aspects in which the cleavage site has at least 80, 81, 82, 83, 84, 85, 86, 87, 88, 89, 90, 91, 92, 93, 94, 95, 96, 97, 98, 99, or 100% (or any derivable range therein) sequence identity to SEQ ID NO:24 are also contemplated.

The CAR of the disclosure may comprise or further comprise a torsional linker between the transmembrane domain and the cytoplasmic region. In some aspects, the torsional linker comprises or consists of 1, 2, 3, 4, 5, 6, 7, 8, 9, or 10 amino acid residues (or any derivable range therein). In some aspects, the amino acid residues comprise or consist of alanine residues. In some aspects, the torsional linker comprises at least, at most, or exactly 1, 2, 3, 4, 5, 6, 7, 8, 9, or 10 (or any derivable range therein) alanine residues. In some aspects, the torsional linker comprises at least, at most, or exactly 1, 2, 3, 4, 5, 6, 7, 8, 9, or 10 (or any derivable range therein) contiguous alanine residues. In some aspects, the torsional linker consists of 2 or 4 alanine residues. In some aspects, the torsional linker comprises at least, at most, or exactly 1, 2, 3, 4, 5, 6, 7, 8, 9, or 10 (or any derivable range therein) contiguous alanine residues. In some aspects, the torsional linker consists of 2 alanine residues.

The CAR of the disclosure may include a peptide spacer between the antigen binding domains and the transmembrane domain. Similarly, the second CAR of the disclosure may include a peptide spacer is between the antigen binding domains and the second transmembrane domain of the second CAR. The peptide spacer or second peptide spacer may comprise an IgG4 hinge region. In some aspects, the IgG4 hinge region comprises a polypeptide having an amino acid sequence with at least 80% sequence identity to SEQ ID NO:12. In some aspects, the IgG4 hinge region comprises a polypeptide having an amino acid sequence with or with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:12. In some aspects, the IgG4 hinge region comprises a polypeptide having the amino acid sequence of SEQ ID NO:12. In some aspects, the IgG4 hinge region comprises a polypeptide having an amino acid sequence with at least 80% sequence identity to SEQ ID NO:5. In some aspects, the IgG4 hinge region comprises a polypeptide having an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:5. In some aspects, the IgG4 hinge region comprises a polypeptide having the amino acid sequence of SEQ ID NO:5. In some aspects, the peptide spacer or second peptide spacer comprises or further comprises an IgG4 CH2 and CH3 region. In some aspects, the peptide spacer or second peptide spacer comprises or further comprises an IgG4 CH2 and CH3 region. In some aspects, the IgG4 CH2 and CH3 region comprises a polypeptide having an amino acid sequence with at least 80% sequence identity to SEQ ID NO:37. In some aspects, the IgG4 CH2 and CH3 region comprises a polypeptide having an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:37. In some aspects, the IgG4 CH2 and CH3 region comprises a polypeptide having the amino acid sequence of SEQ ID NO:37. In some aspects, the CH2 region comprises L235E and/or N297Q substitutions. The peptide spacer may be between 8 and 1000 amino acids in length. In some aspects, the peptide spacer is between 8 and 500 amino acids in length. In some aspects, the peptide spacer is between 100-300 amino acids in length. In some aspects, the peptide spacer has fewer than 100 amino acids. In some aspects, the peptide spacer is at least, at most, or exactly, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 amino acids (or any derivable range therein).

The transmembrane domain or second transmembrane domain may comprise the transmembrane domain from the CD28 protein. In some aspects, the transmembrane domain or second transmembrane domain comprises a transmembrane domain having an amino acid sequence with at least 80% sequence identity to SEQ ID NO:6. In some aspects, the transmembrane domain or second transmembrane domain comprises a transmembrane domain having an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:6. In some aspects, the transmembrane domain or second transmembrane domain comprises a transmembrane domain having the amino acid sequence of SEQ ID NO:6. In some aspects, the transmembrane domain is an alpha or beta chain of the T cell receptor, CD28, CD3ε (epsilon), CD45, CD4, CD5, CD8, CD9, CD16, CD22, CD33, CD37, CD64, CD80, CD86, CD123, CD134, CD137 or CD154 transmembrane domain.

The co-stimulatory region or second co-stimulatory region in the peptides and CARs described herein may comprise the co-stimulatory region from the 4-1BB protein or from the CD28 protein. In some aspects, the co-stimulatory region or second co-stimulatory region comprises a co-stimulatory region having an amino acid sequence with at least 80% sequence identity to SEQ ID NO:7, 14, or 18. In some aspects, the co-stimulatory region or second co-stimulatory region comprises a co-stimulatory region having an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:7, 14, or 18. In some aspects, the co-stimulatory region or second co-stimulatory region comprises a co-stimulatory region having the amino acid sequence of SEQ ID NO:7, 14, or 18. In some aspects, the cytoplasmic region comprises two costimulatory domains. In some aspects, the one or more costimulatory domain(s) comprise a costimulatory domain from one or more of 4-1BB (CD137), CD28, IL-15Rα, OX40, CD2, CD27, CDS, ICAM-1, LFA-1 (CD11a/CD18), and/or ICOS (CD278). In some aspects, the one or more costimulatory domains comprise a costimulatory domain from CD28 or a costimulatory domain derived from CD28.

The primary intracellular signaling domain or second primary intracellular signaling domain of the polypeptides and CARs described herein may comprise an intracellular signaling domain from the CD3ζ protein. In some aspects, the primary intracellular signaling domain or second primary intracellular signaling domain comprises an intracellular signaling domain having an amino acid sequence with at least 80% sequence identity to SEQ ID NO:8 or 15. In some aspects, the primary intracellular signaling domain or second primary intracellular signaling domain comprises an intracellular signaling domain having an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:8 or 15. In some aspects, the primary intracellular signaling domain or second primary intracellular signaling domain comprises an intracellular signaling domain having the amino acid sequence of SEQ ID NO:8 or 15.

The polypeptides may comprise an amino acid sequence of one of SEQ ID NOS:136-147 or an amino acid sequence having at least 80% sequence identity to one of SEQ ID NOS:136-147. The polypeptides may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:136-147. The polypeptides may comprise an amino acid sequence of one of SEQ ID NOS:1, 9, 13, 16, 17, 19, 21-23, and 25 or an amino acid sequence having at least 80% sequence identity to one of SEQ ID NOS:1, 9, 13, 16, 17, 19, 21-23, and 25. The polypeptides may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:1, 9, 13, 16, 17, 19, 21-23, and 25.

The polypeptides of the disclosure may comprise or further comprise one or more molecular tag(s). In some aspects, the one or more molecular tags comprise FLAG and/or HA tag. The polypeptides of the disclosure may comprise or further comprise one or more signal sequence(s). In some aspects, the signal sequence(s) comprise an amino acid sequence with at least 80% sequence identity to SEQ ID NO:2. In some aspects, the signal sequence(s) comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:2. In some aspects, the signal sequence(s) comprise the amino acid sequence of SEQ ID NO:2. The polypeptides may also exclude a FLAG tag in the CARs of the disclosure.

Aspects of the disclosure also describe nucleic acids encoding the polypeptides and expression vectors comprising the nucleic acid. The expression construct may be a viral vector, such as a retroviral vector or a vector derived from a retrovirus. In some aspects, the viral vector is a lentiviral vector or a vector derived from a lentivirus. Aspects relate to a lentivirus vector comprising a sequence encoding a polypeptide of the disclosure. Aspects also relate to viral particles comprising nucleic acids of the disclosure. In some aspects, the expression vector, such as the viral vector has integrated into the host cell's genome. The cell may be ex vivo. It is also contemplated that the cell is in vivo. Aspects of the disclosure also relate to cells expressing a polypeptide of the disclosure and/or comprising a nucleic acid of the disclosure. Cells of the disclosure may comprise or further comprise a nucleic acid encoding a second CAR. Cells of the disclosure may comprise or further comprise or express a polypeptide comprising a second CAR.

The cells of the disclosure may comprise a a nucleic acid that encodes for or comprise an expressed first polypeptide with a bi-specific CAR comprising an IL13Rα binding region and a TGF-beta binding region and a second polypeptide having a CAR comprising a GD2 binding region. The cells of the disclosure may comprise a a nucleic acid that encodes for or comprise an expressed first polypeptide with a bi-specific CAR comprising an IL13Rα binding region and a TGF-beta binding region and a second polypeptide having a CAR comprising a EGFRvIII binding region. The cells of the disclosure may comprise a a nucleic acid that encodes for or comprise an expressed first polypeptide with a bi-specific CAR comprising an IL13Rα binding region and a GD2 binding region and a second polypeptide having a CAR comprising a TGF-beta binding region. The cells of the disclosure may comprise a a nucleic acid that encodes for or comprise an expressed first polypeptide with a bi-specific CAR comprising an IL13Rα binding region and a EGFRvIII binding region and a second polypeptide having a CAR comprising a TGF-beta binding region. The cells of the disclosure may comprise a a nucleic acid that encodes for or comprise an expressed first polypeptide with a bi-specific CAR comprising a TGF-beta binding region and a EGFRvIII binding region and a second polypeptide having a CAR comprising an IL13Rα binding region. The cells of the disclosure may comprise a a nucleic acid that encodes for or comprise an expressed first polypeptide with a bi-specific CAR comprising a TGF-beta binding region and a GD2 binding region and a second polypeptide having a CAR comprising an IL13Rα binding region. The cells of the disclosure may comprise a a nucleic acid that encodes for or comprise an expressed first polypeptide with a bi-specific CAR comprising a TGF-beta binding region and a GD2 binding region and a second polypeptide having a bi-specific CAR comprising an IL13Rα binding region and a EGFRvIII binding region. The cells of the disclosure may comprise a a nucleic acid that encodes for or comprise an expressed first polypeptide with a bi-specific CAR comprising a TGF-beta binding region and a EGFRvIII binding region and a second polypeptide having a bi-specific CAR comprising an IL13Rα binding region and a GD2 binding region. The cells of the disclosure may comprise a a nucleic acid that encodes for or comprise an expressed first polypeptide with a bi-specific CAR comprising an IL13Rα binding region and a TGFbeta binding region and a second polypeptide having a bi-specific CAR comprising a EGFRvIII binding region and a GD2 binding region.

Nucleic acids comprising a sequence that encodes the polypeptides disclosed herein, and portions thereof, are provided in aspects. A nucleic acid may comprise RNA or DNA. In certain aspects, the nucleic acid is an expression construct. In some aspects, the expression construct is a vector. In certain aspects, the vector is a viral vector. The viral vector is a retroviral vector or derived from a retrovirus in particular aspects. In some aspects, the retroviral vector comprises a lentiviral vector or is derived from a lentivirus. It is noted that a viral vector is an integrating nucleic acid in certain aspects. Additionally, a nucleic acid may be a molecule involved in gene editing such that a nucleic acid (such as a guide RNA) encoding a CAR is used to incorporate a CAR-coding sequence into a particular locus of the genome, such as the TRAC gene. This may involve a gene editing system such as CRISPR/Cas9. A nucleic acid, polynucleotide, or polynucleotide region (or a polypeptide or polypeptide region) has a certain percentage (for example, 60%, 65%, 70%, 75%, 80%, 85%, 90%, 95%, 98% or 99%—or any range derivable therein) of “sequence identity” or “homology” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. It is contemplated that a nucleic acid may have such sequence identity or homology to any nucleic acid SEQ ID NO provided herein.

In other aspects, there is a cell or a population of cells comprising a nucleic acid that encodes all or part of any polypeptide discussed herein. In certain aspects, a cell or population of cells contains within its genome a sequence encoding any of the polypeptides described herein. This includes, but is not limited to, a lentivirus or retrovirus that has integrated into the cell's genome. In some aspects, a cell or population of cells expresses all or part of any CAR discussed herein, including, but not limited to those with the amino acid sequence of any of and/or comprising the amino acid sequence of any of SEQ ID NOS:1-147. Progeny (F1, F2, and beyond) of cells in which a nucleic acid encoding a polypeptide was introduced are included in the cells or populations of cells disclosed herein. In some aspects, a cell or population of cells is a T cell, a natural killer (NK) cell, a natural killer T cell (NKT), an invariant natural killer T cell (iNKT), stem cell, lymphoid progenitor cell, peripheral blood mononuclear cell (PBMC), hematopoietic stem and progenitor cell (HSPC), hematopoietic stem cell (HSC), CD34+ cell, peripheral blood stem cell (PBSC), bone marrow cell, fetal liver cell, embryonic stem cell, cord blood cell, induced pluripotent stem cell (iPS cell). Specific aspects concern a cell that is a T cell or an NK cell. In some aspects, T cell comprises a naïve memory T cell. In some aspects, the naïve memory T cell comprises a CD4+ or CD8+ T cell. In some aspects, the cells are a population of cells comprising both CD4+ and CD8+ T cells. In some aspects, the cells are a population of cells comprising naïve memory T cells comprising CD4+ and CD8+ T cells. In some aspects, the T cell comprises a T cell from a population of CD14 depeleted, CD25 depleted, and/or CD62L enriched PBMCs.

In some aspects, the disclosure relates to a cell comprising one or more polypeptides described herein. In some aspects, the cell is an immune cell. In some aspects, the cell is a progenitor cell or stem cell. In some aspects, the progenitor or stem cell is in vitro differentiated into an immune cell. In some aspects, the cell is a T cell. In some aspects, the cell is a CD4+ or CD8+ T cell. In some aspects, the cell is a natural killer cell. In some aspects, the cell is ex vivo. The term immune cells includes cells of the immune system that are involved in defending the body against both infectious disease and foreign materials. Immune cells may include, for example, neutrophils, eosinophils, basophils, natural killer cells, lymphocytes such as B cells and T cells, and monocytes. T cells may include, for example, CD4+, CD8+, T helper cells, cytotoxic T cells, γδ T cells, regulatory T cells, suppressor T cells, and natural killer T cells. In a specific aspect, the T cell is a regulatory T cell.

In some aspects, the population of cells comprise 10³-10⁸ cells. In some aspects, the the population is about, is at least about, or is at most about 10², 10³, 10⁴, 10⁵, 10⁶, 10⁷, 10⁸, 10⁹, 10¹⁰, 10¹¹, 10¹² cells (or any range derivable therein). In certain aspects, cells are autologous with respect to a patient who will receive them. In other aspects, cells are not autologous and may be allogenic.

In some aspects of the disclosure, method aspects relate to wherein the cell is infected with a virus encoding a polypeptide of the disclosure. Further aspects relate to a virus comprising a polypeptide and/or nucleic acid of the disclosure. In some aspects, the virus comprises lentivirus or a lentiviral-derived virus or vector. In some aspects, the cell is a T cell, a natural killer (NK) cell, a natural killer T cell (NKT), an invariant natural killer T cell (iNKT), stem cell, lymphoid progenitor cell, peripheral blood mononuclear cell (PBMC), bone marrow cell, fetal liver cell, embryonic stem cell, cord blood cell, induced pluripotent stem cell (iPS cell). In some aspects, the cell is a T cell or an NK cell. In some aspects, the T cell comprises a naïve memory T cell. In some aspects, the naïve memory T cell comprises a CD4+ or CD8+ T cell. In some aspects, the cell is not yet a T cell or NK cell, the method further comprising culturing the cell under conditions that promote the differentiation of the cell into a T cell or an NK cell. In some aspects, the methods further comprise culturing the cell under conditions to expand the cell before and or after introducing the nucleic acid into the cell. In some aspects, the cell is cultured with serum-free medium.

Additional methods concern treating a patient with glioblastoma comprising administering to the patient an effective amount of the composition comprising a cell population expressing a polypeptide of the disclosure. In some aspects, a patient has relapsed or recurrent cancer. Further aspects include a step of administering an additional therapy to the patient. The patient may be one that has been diagnosed with glioblastoma and/or a glioblastoma that has GD+ or EGFRvIII+ cells, as described herein. The patient may be one that has been determined to have glioblastoma and/or a glioblastoma that has GD+ or EGFRvIII+ cells, as described herein. In some aspects, the subject is one that is at risk of having glioblastoma and/or GD2+ or EGFRvIII+ glioblastoma. In some aspects, the patient has been previously treated to the cancer. In some aspects, the patient has been determined to be resistant to the previous treatment. The previous treatment may be a cancer therapeutic described herein, such as those described as additional therapies. Further aspects include a step of administering chemotherapy and/or radiation to the patient. In some aspects, the additional therapy comprises an immunotherapy. In some aspects, the additional therapy comprises an additional therapy described herein. In some aspects, the immunotherapy comprises immune checkpoint inhibitor therapy. In some aspects, the immunotherapy comprises an immunotherapy described herein. In some aspects, the immune checkpoint inhibitor therapy comprises a PD-1 inhibitor and/or CTLA-4 inhibitor. In some aspects, the immune checkpoint inhibitor therapy comprises one or more inhibitors of one or more immune checkpoint proteins described herein.

In some aspects, the cancer comprises a GD2+ cancer, wherein a GD2+ cancer is one that comprises GD2+ cells or comprises at least 0.5, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, or 90% GD2+ cancer cells in a population of tumor cells.

In some aspects, the cancer comprises a EGFRvIII+ cancer, wherein a EGFRvIII+ cancer is one that comprises EGFRvIII+ cells or comprises at least 0.5, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, or 90% EGFRvIII+ cancer cells in a population of tumor cells.

In some aspects, the cancer comprises glioblastoma. The CAR polypeptides of the current disclosure may have a region, domain, linker, spacer, or other portion thereof that comprises or consists of an amino acid sequence that is at least, at most, or exactly 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical (or any range derivable therein) to all or a portion of the amino acid sequences described herein. In certain aspects, a CAR polypeptide comprises or consists of an amino acid sequence that is, is at least, is at most, or exactly 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% identical (or any range derivable therein) to any one of SEQ ID NOS:1-147.

In aspects of the disclosure the method may comprise stimulating an immune response, wherein stimulating an immune response comprises increasing expression and/or secretion of immune stimulating cytokines and/or molecules. In some aspects, the immune stimulating cytokines and/or molecules are one or more of TNF-α, IFN-β, IFN-γ, IL-1, IL-2, IL-4, IL-6, IL-8, IL-10, IL-12, IL-18 and granulocyte-macrophage colony stimulating factor. In some aspects, stimulating an immune response comprises increasing proliferation of immune cells. The immune cells may be T cells. In some aspects, the cells are ex vivo. The cell may also be in vivo in a subject in need of immune stimulation. The subject may be one that produces endogenous TGF-β and/or an excess of endogenous TGF-β. An increase in expression or proliferation as described herein may be at least, at most, or exactly 1, 2, 3, 4, 5, 6, 7, 8, 9, 10, 50, 100, 200, 300, 500, or 1000 fold increase over a base-line expression level such as a control (non-disease, non-TGF-β or non-antigen binding polypeptide control). In some aspects, the methods are for treating a person with an indication, wherein the indication is characterized by a pathogenic level of expression of TGF-β.

The subject may be a mammal, such as a human, rat, mouse, or non-human primate. In a particular aspect, the subject is a human. The subject may also be a goat, pig, horse, cat, or dog. The route of administration of the compositions, polypeptides, cells, and nucleic acids of the disclosure may be a route of administration described herein. In some aspects, the compositions are administered intraventricularly, intracerebroventricularly, intratumorally, intravenously, or into a tumor resection cavity. In some aspects, the compositions are formulated for intraventricular, intracerebroventricular, intratumoral, or intravenous administration or for administration into a tumor resection cavity.

In some aspects, the method further comprises administering TGF-β to the subject. In compositions of the disclosure, the composition may comprise 1-50 ng/mL of TGF-β. In some aspects, the composition comprises at least, at most, or about 0.5, 1, 1.5, 2, 2.5, 3, 3.5, 4, 4.5, 5, 5.5, 6, 6.5, 7, 7.5, 8, 8.5, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, or 100 ng/mL of TGF-β (or any range derivable therein).

In some aspects, the composition further comprises IL-2. In some aspects, the composition comprises 20-400 U/mL of IL-2. In some aspects, the composition comprises at least, at most, or about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600 U/mL of IL-2 (or any range derivable therein). In some aspects, the composition further comprises IL-15. In some aspects, the composition comprises 0.1-10 ng/mL of IL-15. In some aspects, the composition comprises at least, at most, or about 0.05, 0.06, 0.07, 0.08, 0.09, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, 3.00, 3.05, 3.10, 3.15, 3.20, 3.25, 3.30, 3.35, 3.40, 3.45, 3.50, 3.55, 3.60, 3.65, 3.70, 3.75, 3.80, 3.85, 3.90, 3.95, 4.00, 4.05, 4.10, 4.15, 4.20, 4.25, 4.30, 4.35, 4.40, 4.45, 4.50, 4.55, 4.60, 4.65, 4.70, 4.75, 4.80, 4.85, 490, 495, 5.00, 5.05, 5.10, 5.15, 5.20, 5.25, 5.30, 5.35, 5.40, 5.45, 5.50, 5.55, 5.60, 5.65, 5.70, 5.75, 580, 5.85, 5.90, 5.95, 6.00, 6.5, 7, 7.5, 8, 8.5, 9, 9.5, 10, 11, 12, 13, 14, 15, 16, 17, 18, 19, 20, 30, 40, or 50 ng/mL of IL-15 (or any range derivable therein). In some aspects, the composition comprises or further comprises IL-7, IL-12, and/or IL-21. In some aspects, the composition comprises at least, at most, or about 0.05, 0.06, 0.07, 0.08, 0.09, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, 3.00, 3.05, 3.10, 3.15, 3.20, 3.25, 3.30, 3.35, 3.40, 3.45, 3.50, 3.55, 3.60, 3.65, 3.70, 3.75, 3.80, 3.85, 3.90, 3.95, 4.00, 4.05, 4.10, 4.15, 4.20, 4.25, 4.30, 4.35, 4.40, 4.45, 4.50, 4.55, 4.60, 4.65, 4.70, 4.75, 4.80, 4.85, 490, 495, 5.00, 5.05, 5.10, 5.15, 5.20, 5.25, 5.30, 5.35, 5.40, 5.45, 5.50, 5.55, 5.60, 5.65, 5.70, 5.75, 580, 5.85, 5.90, 5.95, 6.00, 6.5, 7, 7.5, 8, 8.5, 9, 9.5, 10, 11, 12, 13, 14, 15, 16, 17, 18, 19, 20, 30, 40, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600 pg/mL, ng/mL, μg/mL, or mg/ml of IL-7, IL-12, and/or IL-21 (or any range derivable therein).

In some aspects, the method further comprises contacting the cells with feeder cells. In some aspects, the feeder cells are irradiated. Feeder cells or support cells can include, for example, fibroblasts, mouse embryonic fibroblasts, JK1 cells, SNL 76/7 cells, human fetal skin cells, human fibroblasts, and human foreskin fibroblasts.

In some aspects, the method excludes contacting T cells with feeder cells. In some cases, the excluded feeder cells are from a different animal species as the T cells.

In certain aspects, polypeptides described throughout this disclosure are isolated, meaning they are not found in the cellular milieu. In some cases, they are purified, which means it is mostly if not completely separated from polypeptides having a different amino acid sequence and/or chemical formula.

The present disclosure provides, in some aspects, a method for treating a subject with cancer comprising administering to the subject an effective amount of a population of cells or pharmaceutical composition comprising a chimeric polypeptide or nucleic acid encoding a chimeric polypeptide.

“Treatment” or “Treating” may refer to any treatment of a disease in a mammal, including: (i) suppressing the disease, that is, causing the clinical symptoms of the disease not to develop by administration of a protective composition after the inductive event but prior to the clinical appearance or reappearance of the disease; (ii) inhibiting the disease, that is, arresting the development of clinical symptoms by administration of a protective composition after their initial appearance; and/or (iii) relieving the disease, that is, causing the regression of clinical symptoms by administration of a protective composition after their initial appearance. In some aspects, the treatment may exclude prevention of the disease.

Use of the one or more sequences or compositions may be employed based on any of the methods described herein. Other aspects and embodiments are discussed throughout this application. Any embodiment or aspect discussed with respect to one aspect of the disclosure applies to other aspects of the disclosure as well and vice versa. For example, any step in a method described herein can apply to any other method. Moreover, any method described herein may have an exclusion of any step or combination of steps. The embodiments in the Example section are understood to be embodiments that are applicable to all aspects of the technology described herein.

It is specifically contemplated that any method, composition, cell, polypeptide, or nucleic acid embodiment or aspect described herein may be used interchangeably and in combination with each other. Furthermore, it is contemplated that embodiments and aspects of the disclosure may specifically exclude an embodiment or aspect described herein.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: FLAG (CAR) Surface Expression (No Antigen Stimulation). Averages of triplicates are shown, with error bars representing ±1 standard deviation. Each set of two bars represents data, from left to right, of the SP and Full IL13R construct.

FIG. 2A-D: (2A) CD69 Activation Marker Expression after 21-hr Stimulation. (2B) CD25 Activation Marker Expression after 21-hr Stimulation. (2C) FLAG (CAR) Surface Expression after 21-hr Stimulation. (2D) FLAG (CAR) Surface Expression after 21-hr Stimulation. PBT106 NS is a tumor line that expresses IL-13Rα2. Averages of triplicates are shown, with error bars representing ±1 standard deviation. Each set of three bars represents the data, from left to right, of 1) media only; 2) 5 ng/mL TGF-β; and 3) 100% IL13Rα2+PBT 106 NS.

FIG. 3A-F: (3A-3B) Viable Tumor Count after 92-hr Coincubation. (3C-3D) Viable T-cell Count after 92-hr Coincubation (3E-3F) CTV Dilution among T Cells after 92-hr Coincubation. T cells were stained with CellTrace Violet (CTV) dye, which dilutes with each T-cell division. Therefore, the lower the CTV MFI, the more times the T cells have divided. Averages of triplicates are shown, with error bars representing ±1 standard deviation. Each set of 16 bars represents the data, from left to right, of 1) SP-IL13Rα2.BBz; 2) Full-IL13Rα2.BBz; 3) SP-IL13Rα2/TGF-β.BBz; 4) Full-IL13Rα2/TGF-β.BBz; 5) SP-IL13Rα2.BBz KR; 6) Full-IL13Rα2.BBz KR; 7) SP-IL13Rα2/TGF-β.BBz KR; 8) SP-IL13Rα2.28z; 9) Full-IL13Rα2.28z; 10) SP-IL13Rα2/TGF-β.28z; 11) Full-IL13Rα2/TGF-β.28z; 12) SP-IL13Rα2/TGF-β.BBz+GD2.AA.28z; 13) Full-IL13Rα2/TGF-β.BBz+GD2.AA.28z; 14) SP-IL13Rα2.BBz+ TGF-β DNR; 15) Full-IL13Rα2.BBz+ TGF-β DNR; and 16) scFv-less CAR.

FIG. 4. NOD/scid/γ^{-/-} (NSG) mice were intracranially engrafted with 2.5×10\hat{ }5 PBT106 glioblastoma multiforme (GBM) neurosphere cells that stably express firefly luciferase. Tumor-bearing mice were treated with 0.5×10\hat{ }6 CAR+ cells 7 days after tumor injection. Tumor progression was monitored by bioluminescence imaging. Each trace represents one mouse, with “x” marking time of sacrifice for mice that reached the humane end point. Survival is shown in Kaplan-Meier curve.

### DETAILED DESCRIPTION

### I. Definitions

The peptides of the disclosure relate to peptides comprising chimeric antigen receptors, or CARs. CARs are engineered receptors, which are capable of grafting an arbitrary specificity onto an immune effector cell. In some cases, these receptors are used to graft the specificity of a monoclonal antibody onto a T cell. The receptors are called chimeric because they are composed of parts from different sources.

The terms “protein,” “polypeptide,” and “peptide” are used interchangeably herein when referring to a gene product.

“Homology,” or “identity” refers to sequence similarity between two peptides or between two nucleic acid molecules. Identity can be determined by comparing a position in each sequence which may be aligned for purposes of comparison. When a position in the compared sequence is occupied by the same base or amino acid, then the molecules share sequence identity at that position. A degree of identity between sequences is a function of the number of matching or homologous positions shared by the sequences. An “unrelated” or “non-homologous” sequence shares less than 60% identity, less than 50% identity, less than 40% identity, less than 30% identity, or less than 25% identity, with one of the sequences of the current disclosure.

The terms “amino proximal,” “N-terminus,” “amino terminus,” and the like as used herein are used to refer to order of the regions of the polypeptide. Furthermore, when something is N-terminal or amino proximal to a region it is not necessarily at the terminus (or end) of the entire polypeptide, but just at the N-terminus of the region or domain. Similarly, the terms “carboxy proximal,” “C-terminus,” “carboxy terminus,” and the like as used herein is used to refer to order of the regions of the polypeptide, and when something is C-terminal or carboxy proximal to a region it is not necessarily at the terminus (or end) of the entire polypeptide, but just at the C-terminus of the region or domain.

The terms “polynucleotide,” “nucleic acid,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment and aspect of this invention that is a polynucleotide encompasses both the double-stranded form and each of two complementary single-stranded forms known or predicted to make up the double-stranded form.

A “gene,” “polynucleotide,” “coding region,” “sequence,” “segment,” “fragment,” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “antibody” includes monoclonal antibodies, polyclonal antibodies, dimers, multimers, multispecific antibodies and antibody fragments that may be human, mouse, humanized, chimeric, or derived from another species. A “monoclonal antibody” is an antibody obtained from a population of substantially homogeneous antibodies that is being directed against a specific antigenic site.

“Antibody or functional fragment thereof” means an immunoglobulin molecule that specifically binds to, or is immunologically reactive with a particular antigen or epitope, and includes both polyclonal and monoclonal antibodies. The term antibody includes genetically engineered or otherwise modified forms of immunoglobulins, such as intrabodies, peptibodies, chimeric antibodies, fully human antibodies, humanized antibodies, and heteroconjugate antibodies (e.g., bispecific antibodies, diabodies, triabodies, and tetrabodies). The term functional antibody fragment includes antigen binding fragments of antibodies, including e.g., Fab′, F(ab′)2, Fab, Fv, rlgG, and scFv fragments. The term scFv refers to a single chain Fv antibody in which the variable domains of the heavy chain and of the light chain of a traditional two chain antibody have been joined to form one chain.

As used herein, the term “binding affinity” refers to the equilibrium constant for the reversible binding of two agents and is expressed as a dissociation constant (Kd). Binding affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more (or any derivable range therein), than the binding affinity of an antibody for unrelated amino acid sequences. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges.

“Individual, “subject,” and “patient” are used interchangeably and can refer to a human or non-human.

The terms “lower,” “reduced,” “reduction,” “decrease,” or “inhibit” are all used herein generally to mean a decrease by a statistically significant amount. However, for avoidance of doubt, “lower,” “reduced,” “reduction, “decrease,” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased,” “increase,” “enhance,” or “activate” are all used herein to generally mean an increase by a statically significant amount; for the avoidance of any doubt, the terms “increased,” “increase,” “enhance,” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

### II. Polypeptides

A. Signal Peptide

Polypeptides of the present disclosure may comprise a signal peptide. A “signal peptide” refers to a peptide sequence that directs the transport and localization of the protein within a cell, e.g., to a certain cell organelle (such as the endoplasmic reticulum) and/or the cell surface. In some aspects, a signal peptide directs the nascent protein into the endoplasmic reticulum. This is essential if a receptor is to be glycosylated and anchored in the cell membrane. Generally, the signal peptide natively attached to the amino-terminal most component is used (e.g. in an scFv with orientation light chain-linker-heavy chain, the native signal of the light-chain is used).

In some aspects, the signal peptide is cleaved after passage of the endoplasmic reticulum (ER), i.e., is a cleavable signal peptide. In some aspects, a restriction site is at the carboxy end of the signal peptide to facilitate cleavage.

B. Antigen Binding Domain

Polypeptides of the present disclosure may comprise one or more antigen binding domains. An “antigen binding domain” describes a region of a polypeptide capable of binding to an antigen under appropriate conditions. In some aspects, an antigen binding domain is a single-chain variable fragment (scFv) based on one or more antibodies (e.g., CD20 antibodies). In some aspects, an antigen binding domain comprise a variable heavy (VH) region and a variable light (VL) region, with the VH and VL regions being on the same polypeptide. In some aspects, the antigen binding domain comprises a linker between the VH and VL regions. A linker may enable the antigen binding domain to form a desired structure for antigen binding.

The variable regions of the antigen-binding domains of the polypeptides of the disclosure can be modified by mutating amino acid residues within the VH and/or VL CDR 1, CDR 2 and/or CDR 3 regions to improve one or more binding properties (e.g., affinity) of the antibody. The term “CDR” refers to a complementarity-determining region that is based on a part of the variable chains in immunoglobulins (antibodies) and T cell receptors, generated by B cells and T cells respectively, where these molecules bind to their specific antigen. Since most sequence variation associated with immunoglobulins and T cell receptors is found in the CDRs, these regions are sometimes referred to as hypervariable regions. Mutations may be introduced by site-directed mutagenesis or PCR-mediated mutagenesis and the effect on antibody binding, or other functional property of interest, can be evaluated in appropriate in vitro or in vivo assays. Preferably conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

It is also contemplated that the antigen binding domain may be multi-specific or multivalent by multimerizing the antigen binding domain with VH and VL region pairs that bind either the same antigen (multi-valent) or a different antigen (multi-specific).

The binding affinity of the antigen binding region, such as the variable regions (heavy chain and/or light chain variable region), or of the CDRs may be at least 10⁻⁵M, 10⁻⁶M, 10⁻⁷M, 10⁻⁸M, 10⁻⁹M, 10⁻¹⁰M, 10⁻¹¹M, 10⁻¹²M, or 10⁻¹³M. In some aspects, the KD of the antigen binding region, such as the variable regions (heavy chain and/or light chain variable region), or of the CDRs may be at least 10⁻⁵M, 10⁻⁶M, 10⁻⁷M, 10⁻⁸M, 10⁻⁹M, 10⁻¹⁰M, 10⁻¹¹M, 10⁻¹²M, or 10⁻¹³M (or any derivable range therein).

Binding affinity, KA, or KD can be determined by methods known in the art such as by surface plasmon resonance (SRP)-based biosensors, by kinetic exclusion assay (KinExA), by optical scanner for microarray detection based on polarization-modulated oblique-incidence reflectivity difference (OI-RD), or by ELISA.

In some aspects, the polypeptide comprising the humanized binding region has equal, better, or at least 80, 81, 82, 83, 84, 85, 86, 87, 88, 89, 90, 91, 92, 93, 94, 95, 96, 97, 98, 99, 100, 101, 102, 103, 104, 104, 106, 106, 108, 109, 110, 115, or 120% binding affinity and/or expression level in host cells, compared to a polypeptide comprising a non-humanized binding region, such as a binding region from a mouse.

In some aspects, the framework regions, such as FR1, FR2, FR3, and/or FR4 of a human framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a mouse framework.

In some aspects, the framework regions, such as FR1, FR2, FR3, and/or FR4 of a mouse framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a human framework.

The substitution may be at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 of FR1, FR2, FR3, or FR4 of a heavy or light chain variable region.

C. Peptide Spacer

A peptide spacer, such as an extracellular spacer may link an antigen-binding domain to a transmembrane domain. In some aspects, a peptide spacer is flexible enough to allow the antigen-binding domain to orient in different directions to facilitate antigen binding. In one aspect, the spacer comprises the hinge region from IgG. In some aspects, the spacer comprises or further comprises the CH2CH3 region of immunoglobulin and portions of CD3. In some aspects, the CH2CH3 region may have L235E/N297Q or L235D/N297Q modifications, or at least 75%, at least 80%, at least 85%, at least 90%, at least 95%, at least 98%, or 100% amino acid sequence identity of the CH2CH3 region. In some aspects, the spacer is from IgG4. An extracellular spacer may comprise a hinge region.

As used herein, the term “hinge” refers to a flexible polypeptide connector region (also referred to herein as “hinge region”) providing structural flexibility and spacing to flanking polypeptide regions and can consist of natural or synthetic polypeptides. A “hinge” derived from an immunoglobulin (e.g., IgG1) is generally defined as stretching from Glu216 to Pro230 of human IgG1 (Burton (1985) Molec. Immunol., 22: 161-206). Hinge regions of other IgG isotypes may be aligned with the IgG1 sequence by placing the first and last cysteine residues forming inter-heavy chain disulfide (S-S) bonds in the same positions. The hinge region may be of natural occurrence or non-natural occurrence, including but not limited to an altered hinge region as described in U.S. Pat. No. 5,677,425, incorporated by reference herein. The hinge region can include a complete hinge region derived from an antibody of a different class or subclass from that of the CH1 domain. The term “hinge” can also include regions derived from CD8 and other receptors that provide a similar function in providing flexibility and spacing to flanking regions.

The extracellular spacer can have a length of at least, at most, or exactly 4, 5, 6, 7, 8, 9, 10, 12, 15, 16, 17, 18, 19, 20, 20, 25, 30, 35, 40, 45, 50, 75, 100, 110, 119, 120, 130, 140, 150, 160, 170, 180, 190, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, 290, 300, 325, 350, or 400 amino acids (or any derivable range therein). In some aspects, the extracellular spacer consists of or comprises a hinge region from an immunoglobulin (e.g. IgG). Immunoglobulin hinge region amino acid sequences are known in the art; see, e.g., Tan et al. (1990) Proc. Natl. Acad. Sci. USA 87: 162; and Huck et al. (1986) Nucl. Acids Res.

The length of an extracellular spacer may have effects on the CAR's signaling activity and/or the CAR-T cells' expansion properties in response to antigen-stimulated CAR signaling. In some aspects, a shorter spacer such as less than 50, 45, 40, 30, 35, 30, 25, 20, 15, 14, 13, 12, 11, or 10 amino acids is used. In some aspects, a longer spacer, such as one that is at least 50, 60, 70, 80, 90, 100, 110, 120, 130, 140, 150, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, or 290 amino acids may have the advantage of increased expansion in vivo or in vitro.

As non-limiting examples, an immunoglobulin hinge region can include one of the following amino acid sequences:

TABLE
Exemplary Hinge Regions

| SEQUENCE | SEQ ID NO: |
|---|---|
| DKTHT | 70 |
| CPPC | 71 |
| CPEPKSCDTPPPCPR | 72 |
| ELKTPLGDTTHT | 73 |
| KSCDKTHTCP | 74 |
| KCCVDCP | 75 |
| KYGPPCP | 76 |
| EPKSCDKTHTCPPCP | 77 |
| ELKTPLGDTTH | 78 |
| TCPRCP | |
| SPNMVPHAHHAQ | 79 |
| ESKYGPPCPPCP | 80 |
| EPKSCDKTYTCPPCP | 81 |

The extracellular spacer can comprise an amino acid sequence of a human IgG1, IgG2, IgG3, or IgG4, hinge region. The extracellular spacer may also include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO:81).

The extracellular spacer can comprise an amino acid sequence derived from human CD8; e.g., the hinge region can comprise the amino acid sequence: TTTPAPRPPTPAPTIASQPLSLRPEACRPAAGGAVHTRGLDFACD (SEQ ID NO:82), or a variant thereof.

The extracellular spacer may comprise or further comprise a CH2 region. An exemplary CH2 region is APEFEGGPSVFLFPPKPKDTLMISRTPEVTCVVVDVS QEDPEVQFNWYVDGVEVHNA KTKPREEQFQSTYRVVSVLTVLHQDWLNGKEYKCKVSNKGLPSSIEKTISKAK (SEQ ID NO:83). The extracellular spacer may comprise or further comprise a CH3 region. An exemplary CH3 region is GQPREPQVYTLPPSQEEMTKNQVSLTCLVKGFYPSDIAVEWESNGQPENNYKTTPPV LDSDGSFFLYSRLTVDKSRWQEGNVFSCSVMHEALHNHYTQKSLSLSLGK (SEQ ID NO:84).

When the extracellular spacer comprises multiple parts, there may be anywhere from 0-50 amino acids in between the various parts. For example, there may be at least, at most, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 amino acids (or any derivable range therein) between the hinge and the CH2 or CH3 region or between the CH2 and CH3 region when both are present. In some aspects, the extracellular spacer consists essentially of a hinge, CH2, and/or CH3 region, meaning that the hinge, CH2, and/or CH3 region is the only identifiable region present and all other domains or regions are excluded, but further amino acids not part of an identifiable region may be present.

D. Transmembrane Domain

Polypeptides of the present disclosure may comprise a transmembrane domain. In some aspects, a transmembrane domain is a hydrophobic alpha helix that spans the membrane. Different transmembrane domains may result in different receptor stability.

In some aspects, the transmembrane domain is interposed between the extracellular spacer and the cytoplasmic region. In some aspects, the transmembrane domain is interposed between the extracellular spacer and one or more costimulatory regions. In some aspects, a linker is between the transmembrane domain and the one or more costimulatory regions.

Any transmembrane domain that provides for insertion of a polypeptide into the cell membrane of a eukaryotic (e.g., mammalian) cell may be suitable for use. In some aspects, the transmembrane domain is derived from CD28, CD8, CD4, CD3-zeta, CD134, or CD7.

Exemplary transmembrane domains useful in any of the aspects of the disclosure include those in the table below:

TABLE
Exemplary transmembrane domain sequences

| | | SEQ ID |
|---|---|---|
| Description | Sequence | NO: |
| CD28- | FWVLVVVGGVLACYSLLVTVA | 85 |
| derived | FIIFWV | |
| CD8 beta | LGLLVAGVLVLLVSLGVAIHL | 86 |
| derived | CC | |
| CD4 | ALIVLGGVAGLLLFIGLGIF | 87 |
| derived | FCVRC | |
| CD3 zeta | LCYLLDGILFIYGVILTALF | 88 |
| derived | LRV | |
| CD28 | WVLVVVGGVLACYSLLVTVA | 89 |
| derived | FIIFWV | |
| CD134 | VAAILGLGLVLGLLGPLAILL | 90 |
| (OX40) | ALYLL | |
| derived | | |
| CD7 | ALPAALAVISFLLGLGLGVAC | 91 |
| derived | VLA | |

E. Cytoplasmic Region

After antigen recognition, receptors of the present disclosure may cluster and a signal transmitted to the cell through the cytoplasmic region. In some aspects, the costimulatory domains described herein are part of the cytoplasmic region. In some aspects, the cytoplasmic region comprises an intracellular signaling domain. An intracellular signaling domain may comprise a primary signaling domain and one or more costimulatory domains.

Cytoplasmic regions and/or costimulatory regions suitable for use in the polypeptides of the disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation by way of binding of the antigen to the antigen binding domain. In some aspects, the cytoplasmic region includes at least one (e.g., one, two, three, four, five, six, etc.) ITAM motif as described herein. In some aspects, the cytoplasmic region includes DAP10/CD28 type signaling chains.

Cytoplasmic regions suitable for use in the polypeptides of the disclosure include immunoreceptor tyrosine-based activation motif (ITAM)-containing intracellular signaling polypeptides. An ITAM motif is YX1X2(L/I), where X1 and X2 are independently any amino acid. In some cases, the cytoplasmic region comprises 1, 2, 3, 4, or 5 ITAM motifs. In some cases, an ITAM motif is repeated twice in an endodomain, where the first and second instances of the ITAM motif are separated from one another by 6 to 8 amino acids, e.g., (YX1X2(L/I))(X3)n(YX1X2(L/I)), where n is an integer from 6 to 8, and each of the 6-8 X3 can be any amino acid.

A suitable cytoplasmic region may be an ITAM motif-containing portion that is derived from a polypeptide that contains an ITAM motif. For example, a suitable cytoplasmic region can be an ITAM motif-containing domain from any ITAM motif-containing protein. Thus, a suitable endodomain need not contain the entire sequence of the entire protein from which it is derived. Examples of suitable ITAM motif-containing polypeptides include, but are not limited to: DAP12, DAP10, FCER1G (Fc epsilon receptor I gamma chain); CD3D (CD3 delta); CD3E (CD3 epsilon); CD3G (CD3 gamma); CD3-zeta; and CD79A (antigen receptor complex-associated protein alpha chain).

Exemplary cytoplasmic regions are known in the art. The cytoplasmic regions shown below also provide examples of regions that may be incorporated in a CAR of the disclosure:

In some aspects, a suitable cytoplasmic region can comprise an ITAM motif-containing portion of the full length DAP12 amino acid sequence. In some aspects, the cytoplasmic region is derived from FCER1G (also known as FCRG; Fc epsilon receptor I gamma chain; Fc receptor gamma-chain; fc-epsilon Rl-gamma; fcRgamma; fceRI gamma; high affinity immunoglobulin epsilon receptor subunit gamma; immunoglobulin E receptor, high affinity, gamma chain; etc.). In some aspects, a suitable cytoplasmic region can comprise an ITAM motif-containing portion of the full length FCER1G amino acid sequence.

In some aspects, the cytoplasmic region is derived from T cell surface glycoprotein CD3 delta chain (also known as CD3D; CD3-DELTA; T3D; CD3 antigen, delta subunit; CD3 delta; CD3δ; CD3d antigen, delta polypeptide (TiT3 complex); OKT3, delta chain; T cell receptor T3 delta chain; T cell surface glycoprotein CD3 delta chain; etc.). In some aspects, a suitable cytoplasmic region can comprise an ITAM motif-containing portion of the full length CD3 delta amino acid sequence. In some aspects, the cytoplasmic region is derived from T cell surface glycoprotein CD3 epsilon chain (also known as CD3e, CD3ε; T cell surface antigen T3/Leu-4 epsilon chain, T cell surface glycoprotein CD3 epsilon chain, AI504783, CD3, CD3-epsilon, T3e, etc.). In some aspects, a suitable cytoplasmic region can comprise an ITAM motif-containing portion of the full length CD3 epsilon amino acid sequence. In some aspects, the cytoplasmic region is derived from T cell surface glycoprotein CD3 gamma chain (also known as CD3G, CD3γ, T cell receptor T3 gamma chain, CD3-GAMMA, T3G, gamma polypeptide (TiT3 complex), etc.). In some aspects, a suitable cytoplasmic region can comprise an ITAM motif-containing portion of the full length CD3 gamma amino acid sequence. In some aspects, the cytoplasmic region is derived from T cell surface glycoprotein CD3 zeta chain (also known as CD3Z, CD3, T cell receptor T3 zeta chain, CD247, CD3-ZETA, CD3H, CD3Q, T3Z, TCRZ, etc.). In some aspects, a suitable cytoplasmic region can comprise an ITAM motif-containing portion of the full length CD3 zeta amino acid sequence.

In some aspects, the cytoplasmic region is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In some aspects, a suitable cytoplasmic region can comprise an ITAM motif-containing portion of the full length CD79A amino acid sequence.

Specific exemplary cytoplasmic regions are known in the art and further shown in the table below.

TABLE
Cytoplasmic Regions

| | SEQ |
|---|---|
| | ID |
| SEQUENCE | NO: |
| MGGLEPCSRLLLLPLLLAVSGLRPV | 92 |
| QAQAQSDCSCSTVSPGVLAGIVMGD | |
| LVLTVLIALAVYFLGRLVPRGRGAA | |
| EAATRKQRITETESPYQELQGQRSD | |
| VYSDLNTQRPYYK | |
| MGGLEPCSRLLLLPLLLAVSGLRPV | 93 |
| QAQAQSDCSCSTVSPGVLAGIVMGD | |
| LVLTVLIALAVYFLGRLVPRGRGAA | |
| EATRKQRITETESPYQELQGQRSDV | |
| YSDLNTQRPYYK | |
| MGGLEPCSRLLLLPLLLAVSDCSCS | 94 |
| TVSPGVLAGIVMGDLVLTVLIALAV | |
| YFLGRLVPRGRGAAEAATRKQRITE | |
| TESPYQELQGQRSDVYSDLNTQRPY | |
| YK | |
| MGGLEPCSRLLLLPLLLAVSDCSCS | 95 |
| TVSPGVLAGIVMGDLVLTVLIALAV | |
| YFLGRLVPRGRGAAEATRKQRITET | |
| ESPYQELQGQRSDVYSDLNTQRPYY | |
| K | |
| MIPAVVLLLLLLVEQAAALGEPQLC | 96 |
| YILDAILFLYGIVLTLLYCRLKIQV | |
| RKAAITSYEKSDGVYTGLSTRNQET | |
| YETLKHEKPPQ | |
| DGVYTGLSTRNQETYETLKHE | 97 |
| MEHSTFLSGLVLATLLSQVSPFKIP | 98 |
| IEELEDRVFVNCNTSITWVEGTVGT | |
| LLSDITRLDLGKRILDPRGIYRCNG | |
| TDIYKDKESTVQVHYRMCQSCVELD | |
| PATVAGIIVTDVIATLLLALGVFCF | |
| AGHETGRLSGAADTQALLRNDQVYQ | |
| PLRDRDDAQYSHLGGNWARNK | |
| MEHSTFLSGLVLATLLSQVSPFKIP | 99 |
| IEELEDRVFVNCNTSITWVEGTVGT | |
| LLSDITRLDLGKRILDPRGIYRCNG | |
| TDIYKDKESTVQVHYRTADTQALLR | |
| NDQVYQPLRDRDDAQYSHLGGNWAR | |
| NK | |
| DQVYQPLRDRDDAQYSHLGGN | 100 |
| MQSGTHWRVLGLCLLSVGVWGQDGN | 101 |
| EEMGGITQTPYKVSISGTTVILTCP | |
| QYPGSEILWQHNDKNIGGDEDDKNI | |
| GSDEDHLSLKEFSELEQSGYYVCYP | |
| RGSKPEDANFYLYLRARVCENCMEM | |
| DVMSVATIVIVDICITGGLLLLVYY | |
| WSKNRKAKAKPVTRGAGAGGRQRGQ | |
| NKERPPPVPNPDYEPIRKGQRDLYS | |
| GLNQRRI | |
| NPDYEPIRKGQRDLYSGLNQR | 102 |
| MEQGKGLAVLILAIILLQGTLAQSI | 103 |
| KGNHLVKVYDYQEDGSVLLTCDAEA | |
| KNITWFKDGKMIGFLTEDKKKWNLG | |
| SNAKDPRGMYQCKGSQNKSKPLQVY | |
| YRMCQNCIELNAATISGFLFAEIVS | |
| IFVLAVGVYFIAGQDGVRQSRASDK | |
| QTLLPNDQLYQPLKDREDDQYSHLQ | |
| GNQLRRN | |
| DQLYQPLKDREDDQYSHLQGN | 104 |
| MKWKALFTAAILQAQLPITEAQSFG | 105 |
| LLDPKLCYLLDGILFIYGVILTALF | |
| LRVKFSRSADAPAYQQGQNQLYNEL | |
| NLGRREEYDVLDKRRGRDPEMGGKP | |
| RRKNPQEGLYNELQKDKMAEAYSEI | |
| GMKGERRRGKGHDGLYQGLSTATKD | |
| TYDALHMQALPPR | |
| MKWKALFTAAILQAQLPITEAQSFG | 106 |
| LLDPKLCYLLDGILFIYGVILTALF | |
| LRVKFSRSADAPAYQQGQNQLYNEL | |
| NLGRREEYDVLDKRRGRDPEMGGKP | |
| QRRKNPQEGLYNELQKDKMAEAYSE | |
| IGMKGERRRGKGHDGLYQGLSTATK | |
| DTYDALHMQALPPR | |
| NQLYNELNLGRREEYDVLDKR | 107 |
| EGLYNELQKDKMAEAYSEIGMK | 108 |
| DGLYQGLSTATKDTYDALHMQ | 109 |
| MPGGPGVLQALPATIFLLFLLSAVY | 110 |
| LGPGCQALWMHKVPASLMVSLGEDA | |
| HFQCPHNSSNNANVTWWRVLHGNYT | |
| WPPEFLGPGEDPNGTLIIQNVNKSH | |
| GGIYVCRVQEGNESYQQSCGTYLRV | |
| RQPPPRPFLDMGEGTKNRIITAEGI | |
| ILLFCAVVPGTLLLFRKRWQNEKLG | |
| LDAGDEYEDENLYEGLNLDDCSMYE | |
| DISRGLQGTYQDVGSLNIGDVQLEK | |
| P | |
| MPGGPGVLQALPATIFLLFLLSAVY | 111 |
| LGPGCQALWMHKVPASLMVSLGEDA | |
| HFQCPHNSSNNANVTWWRVLHGNYT | |
| WPPEFLGPGEDPNEPPPRPFLDMGE | |
| GTKNRIITAEGIILLFCAVVPGTLL | |
| LFRKRWQNEKLGLDAGDEYEDENLY | |
| EGLNLDDCSMYEDISRGLQGTYQDV | |
| GSLNIGDVQLEKP | |
| ENLYEGLNLDDCSMYEDISRG | 112 |
| FWVLVVVGGVLACYSLLVTVAFIIF | 113 |
| WVRSKRSRLLHSDYMNMTPRRPGPT | |
| RKHYQPYAPPRDFAAYRS | |

F. Costimulatory Region

Non-limiting examples of suitable costimulatory regions, such as those included in the cytoplasmic region, include, but are not limited to, polypeptides from 4-1BB (CD137), CD28, ICOS, OX-40, BTLA, CD27, CD30, GITR, and HVEM.

A costimulatory region may have a length of at least, at most, or exactly 20, 25, 30, 35, 40, 50, 60, 70, 80, 90, 100, 150, 200, or 300 amino acids or any range derivable therein. In some aspects, the costimulatory region is derived from an intracellular portion of the transmembrane protein 4-1BB (also known as TNFRSF9; CD137; CDw137; ILA; etc.). In some aspects, the costimulatory region is derived from an intracellular portion of the transmembrane protein CD28 (also known as Tp44). In some aspects, the costimulatory region is derived from an intracellular portion of the transmembrane protein ICOS (also known as AILIM, CD278, and CVID1). In some aspects, the costimulatory region is derived from an intracellular portion of the transmembrane protein OX-40 (also known as TNFRSF4, RP5-902P8.3, ACT35, CD134, OX40, TXGP1L). In some aspects, the costimulatory region is derived from an intracellular portion of the transmembrane protein BTLA (also known as BTLA1 and CD272). In some aspects, the costimulatory region is derived from an intracellular portion of the transmembrane protein CD27 (also known as S 152, T14, TNFRSF7, and Tp55). In some aspects, the costimulatory region is derived from an intracellular portion of the transmembrane protein CD30 (also known as TNFRSF8, D1S166E, and Ki-1). In some aspects, the costimulatory region is derived from an intracellular portion of the transmembrane protein GITR (also known as TNFRSF18, RP5-902P8.2, AITR, CD357, and GITR-D). In some aspects, the costimulatory region derived from an intracellular portion of the transmembrane protein HVEM (also known as TNFRSF14, RP3-395M20.6, ATAR, CD270, HVEA, HVEM, LIGHTR, and TR2).

Specific exemplary co-stimulatory domains are represented by the amino acid sequences below:

TABLE
Co-stimulatory domains

| | SEQ ID |
|---|---|
| SEQUENCE | NO: |
| KRGRKKLLYIFKQPFMRPVQTTQEE | 7 |
| DGCSCRFPEEEEGGCEL | |
| FWVRSKRSRLLHSDYMNMTPRRPGP | 114 |
| TRKHYQPYAPPRDFAAYRS | |
| TKKKYSSSVHDPNGEYMFMRAVNTA | 115 |
| KKSRLTDVTL | |
| RRDQRLPPDAHKPPGGGSFRTPIQE | 116 |
| EQADAHSTLAKI | |
| CCLRRHQGKQNELSDTAGREINLVD | 117 |
| AHLKSEQTEASTRQNSQVLLSET | |
| GIYDNDPDLCFRMQEGSEVYSNPCL | |
| EENKPGIVYASLNHSVIGPNSRLA | |
| RNVKEAPTEYASICVRS | |
| HQRRKYRSNKGESPVEPAEPCRYSC | 118 |
| PREEEGSTIPIQEDYRKPEPACSP | |
| RRACRKRIRQKLHLCYPVQTSQPKL | 119 |
| ELVDSRPRRSSTQLRSGASVTEPV | |
| AEERGLMSQPLMETCHSVGAAYLES | |
| LPLQDASPAGGPSSPRDLPEPRV | |
| STEHTNNKIEKIYIMKADTVIVGTV | |
| KAELPEGRGLAGPAEPELEEELEA | |
| DHTPHYPEQETEPPLGSCSDVMLSV | |
| EEEGKEDPLPTAASGK | |
| HIWQLRSQCMWPRETQLLLEVPPST | 120 |
| EDARSCQFPEEERGERSAEEKGR | |
| LGDLWV | |
| CVKRRKPRGDVVKVIVSVQRKRQEA | 121 |
| EGEATVIEALQAPPDVTTVAVE | |
| ETIPSFTGRSPNH | |
| RSKRSRGGHSDYMNMTPRRPGPTRK | 18 |
| HYQPYAPPRDFAAYRS | |

G. Detection Peptides

In some aspects, the polypeptides described herein may further comprise a detection peptide. Suitable detection peptides include hemagglutinin (HA; e.g., YPYDVPDYA (SEQ ID NO:122); FLAG (e.g., DYKDDDDK (SEQ ID NO:3)); c-myc (e.g., EQKLISEEDL (SEQ ID NO:123))), and the like. Other suitable detection peptides are known in the art.

H. Peptide Linkers

In some aspects, the polypeptides of the disclosure include peptide linkers (sometimes referred to as a linker). A peptide linker may be used to separate any of the peptide domain/regions described herein. As an example, a linker may be between the signal peptide and the antigen binding domain, between the VH and VL of the antigen binding domain, between the antigen binding domain and the peptide spacer, between the peptide spacer and the transmembrane domain, flanking the costimulatory region or on the N- or C-region of the costimulatory region, and/or between the transmembrane domain and the endodomain. The peptide linker may have any of a variety of amino acid sequences. Domains and regions can be joined by a peptide linker that is generally of a flexible nature, although other chemical linkages are not excluded. A linker can be a peptide of between about 6 and about 40 amino acids in length, or between about 6 and about 25 amino acids in length. These linkers can be produced by using synthetic, linker-encoding oligonucleotides to couple the proteins.

Peptide linkers with a degree of flexibility can be used. The peptide linkers may have virtually any amino acid sequence, bearing in mind that suitable peptide linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any suitable length, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Example flexible linkers include glycine polymers (G)n, glycine-serine polymers (including, for example, (GS)n, (GSGGS)n, (G4S)n and (GGGS)n, where n is an integer of at least one. In some aspects, n is at least, at most, or exactly 1, 2, 3, 4, 5, 6, 7, 8, 9, or 10 (or any derivable range therein). Glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains. Exemplary spacers can comprise amino acid sequences including, but not limited to, GGSG (SEQ ID NO:125), GGSGG (SEQ ID NO:126), GSGSG (SEQ ID NO:127), GSGGG (SEQ ID NO:128), GGGSG (SEQ ID NO:129), GSSSG (SEQ ID NO:124), SEQ ID NO:10, SEQ ID NO:28, and the like. In some aspects, the linker comprises a repeat, such as a contiguous repeat of one or more of SEQ ID NOS:124-129, 10, and 28, such as a linker comprising an amino acid sequence that corresponds to one of SEQ ID NOS: 124-129, 10, and 28 repeated at least, at most, or exactly 2, 3, 4, 5, 6, 7, 8, 9, or 10 times, or any range derivable therein.

In further aspects, the linker comprises (EAAAK)n (SEQ ID NO:130), wherein n is an integer of at least one. In some aspects, n is at least, at most, or exactly 1, 2, 3, 4, 5, 6, 7, 8, 9, or 10 (or any derivable range therein).

I. Therapeutic Controls

In some aspects of the methods and compositions described herein, the CAR molecule is co-expressed with a therapeutic control.

Therapeutic controls regulate cell proliferation, facilitate cell selection (for example selecting cells which express the chimeric antigen receptors of the invention) or a combination thereof. In one aspect, regulating cell proliferation comprises up-regulating cell proliferation to promote cell propagation. In another aspect, regulating cell proliferation comprises down-regulating cell proliferation so as to reduce or inhibit cell propagation. In some aspects, the agents that serve as therapeutic controls may promote enrichment of cells which express the chimeric antigen receptors which may result in a therapeutic advantage. In some aspects, agents which serve as therapeutic controls may biochemically interact with additional compositions so as to regulate the functioning of the therapeutic controls. For example, EGFRt (a therapeutic control) may biochemically interact with cetuximab so as to regulate the function of EGFRt in selection, tracking, cell ablation or a combination thereof.

Exemplary therapeutic controls include truncated epidermal growth factor receptor (EGFRt), chimeric cytokine receptors (CCR) and/or dihydroxyfolate receptor (DHFR) (e.g., mutant DHFR). The polynucleotides encoding the CAR and the therapeutic control(s) may be linked via IRES sequences or via polynucleotide sequences encoding cleavable linkers. The CARs of the invention are constructed so that they may be expressed in cells, which in turn proliferate in response to the presence of at least one molecule that interacts with at least one antigen-specific targeting region, for instance, an antigen. In further aspects, the therapeutic control comprises a cell-surface protein wherein the protein lacks intracellular signaling domains. It is contemplated that any cell surface protein lacking intracellular signaling or modified (e.g. by truncation) to lack intracellular signaling may be used. Further examples of a therapeutic control include truncated LNGFR, truncated CD19, etc., wherein the truncated proteins lack intracellular signaling domains.

“Co-express” as used herein refers to simultaneous expression of two or more genes. Genes may be nucleic acids encoding, for example, a single protein or a chimeric protein as a single polypeptide chain. For example, the CARs of the disclosure may be co-expressed with a therapeutic control (for example truncated epidermal growth factor (EGFRt)), wherein the CAR is encoded by a first polynucleotide chain and the therapeutic control is encoded by a second polynucleotide chain. In an aspect, the first and second polynucleotide chains are linked by a nucleic acid sequence that encodes a cleavable linker The polynucleotides encoding the CAR and the therapeutic control system may be linked by IRES sequences. Alternately, the CAR and the therapeutic control are encoded by two different polynucleotides that are not linked via a linker but are instead encoded by, for example, two different vectors. Further, the CARs of the disclosure may be co-expressed with a therapeutic control and CCR, a therapeutic control and DHFR (for example mutant DHFR) or a therapeutic control and CCR and DHFR (for example mutant DHFR). The CAR, therapeutic control and CCR may be co-expressed and encoded by first, second and third polynucleotide sequences, respectively, wherein the first, second and third polynucleotide sequences are linked via IRES sequences or sequences encoding cleavable linkers (e.g., T2A). Alternately, these sequences are not linked via linkers but instead are encoded via, for example, separate vectors. The CAR, therapeutic control and DHFR (for example mutant DHFR) may be co-expressed and encoded by first, second and fourth polynucleotide sequences, respectively, wherein the first, second and fourth polynucleotide sequences are linked via IRES sequences or via sequences encoding cleavable linkers. Alternately, these sequences are not linked via linkers but instead encoded via, for example, separate vectors. The CAR, therapeutic control, CCR and DHFR (for example mutant DHFR) may be co-expressed and encoded by first, second, third and fourth polynucleotide sequences, respectively, wherein the first, second, third and fourth polynucleotide sequences are linked via IRES sequences or sequences encoding cleavable linkers. Alternately, these sequences are not linked via linkers but instead are encoded via, for example, separate vectors. If the aforementioned sequences are encoded by separate vectors, these vectors may be simultaneously or sequentially transfected.

Further aspects of the therapeutic controls, CAR molecules, and methods of use for the compositions of the disclosure can be found in U.S. Pat. No. 9,447,194, which is herein incorporated by reference for all purposes.

J. Additional Modifications and Polypeptide Enhancements

Additionally, the polypeptides of the disclosure may be chemically modified. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861).

It is contemplated that a region or fragment of a polypeptide of the disclosure may have an amino acid sequence that has, has at least or has at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 or more amino acid substitutions, contiguous amino acid additions, or contiguous amino acid deletions with respect to any of SEQ ID NOS:1-147. Alternatively, a region or fragment of a polypeptide of the disclosure may have an amino acid sequence that comprises or consists of an amino acid sequence that is, is at least, or is at most 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% (or any range derivable therein) identical to any of SEQ ID NOS:1-147. Moreover, in some aspects, a region or fragment comprises an amino acid region of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more contiguous amino acids starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 in any of SEQ ID NOS:1-147 (where position 1 is at the N-terminus of the SEQ ID NO). The polypeptides of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant amino acids or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 600, or more contiguous amino acids, or any range derivable therein, of any of SEQ ID NOS:1-147.

The polypeptides of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 substitutions (or any range derivable therein).

The substitution may be at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 650 of any of SEQ ID NOS:1-147 (or any derivable range therein) and may be a substitution with any amino acid or may be a substitution with a alanine, arginine, asparagine, aspartic acid, cysteine, glutamine, glutamic acid, glycine, histidine, isoleucine, leusine, lysine, methionine, phenylalanine, proline, serine, threonine, tryptophan, tyrosine, or valine.

The polypeptides described herein may be of a fixed length of at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more amino acids (or any derivable range therein).

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria. It is also contemplated that bacteria containing such a variant may be implemented in compositions and methods. Consequently, a protein need not be isolated.

The term “functionally equivalent codon” is used herein to refer to codons that encode the same amino acid, such as the six codons for arginine or serine, and also refers to codons that encode biologically equivalent amino acids.

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity. Structures such as, for example, an enzymatic catalytic domain or interaction components may have amino acid substituted to maintain such function. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity.

In other aspects, alteration of the function of a polypeptide is intended by introducing one or more substitutions. For example, certain amino acids may be substituted for other amino acids in a protein structure with the intent to modify the interactive binding capacity of interaction components. Structures such as, for example, protein interaction domains, nucleic acid interaction domains, and catalytic sites may have amino acids substituted to alter such function. Since it is the interactive capacity and nature of a protein that defines that protein's biological functional activity, certain amino acid substitutions can be made in a protein sequence, and in its underlying DNA coding sequence, and nevertheless produce a protein with different properties. It is thus contemplated by the inventors that various changes may be made in the DNA sequences of genes with appreciable alteration of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is understood that an amino acid can be substituted for another having a similar hydrophilicity value and still produce a biologically equivalent and immunologically equivalent protein.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

In specific aspects, all or part of proteins described herein can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence that encodes a peptide or polypeptide is inserted into an expression vector, transformed or transfected into an appropriate host cell and cultivated under conditions suitable for expression.

One aspect includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide may be employed. The generation of recombinant expression vectors, and the elements included therein, are discussed herein. Alternatively, the protein to be produced may be an endogenous protein normally synthesized by the cell used for protein production.

### III. Cells

Certain aspects relate to cells comprising polypeptides or nucleic acids of the disclosure. In some aspects the cell is an immune cell or a T cell. “T cell” includes all types of immune cells expressing CD3 including T-helper cells, invariant natural killer T (iNKT) cells, cytotoxic T cells, T-regulatory cells (Treg) gamma-delta T cells, natural-killer (NK) cells, and neutrophils. The T cell may refer to a CD4+ or CD8+ T cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell (e.g., peripheral blood stem cell) or progenitor cell obtained from an individual.

### IV. Methods for Modifying Genomic DNA

In certain aspects, the genomic DNA is modified either to include additional mutations, insertions, or deletions, or to integrate certain molecular constructs of the disclosure so that the constructs are expressed from the genomic DNA. In some aspects, a nucleic acid encoding a polypeptide of the disclosure is integrated into the genomic DNA of a cell. In some aspects, a nucleic acid is integrated into a cell via viral transduction, such as gene transfer by lentiviral or retroviral transduction. In some aspects, genomic DNA is modified by integration of nucleic acid encoding a polypeptide of the present disclosure (e.g., a CAR) into the genome of a host cell via a retroviral vector, a lentiviral vector, or an adeno-associated viral vector.

In some aspects, the integration is targeted integration. In some aspects, targeted integration is achieved through the use of a DNA digesting agent/polynucleotide modification enzyme, such as a site-specific recombinase and/or a targeting endonuclease. The term “DNA digesting agent” refers to an agent that is capable of cleaving bonds (i.e. phosphodiester bonds) between the nucleotide subunits of nucleic acids. One specific target is the TRAC (T cell receptor alpha constant) locus. For instance, cells would first be electroporated with a ribonucleoprotein (RNP) complex consisting of Cas9 protein complexed with a single-guide RNA (sgRNA) targeting the TRAC (T cell receptor alpha constant) locus. Fifteen minutes post electroporation, the cells would be treated with AAV6 carrying the HDR template that encodes for the CAR. In another example, double stranded or single stranded DNA comprises the HDR template and is introduced into the cell via electroporation together with the RNP complex.

Therefore, one aspect, the current disclosure includes targeted integration. One way of achieving this is through the use of an exogenous nucleic acid sequence (i.e., a landing pad) comprising at least one recognition sequence for at least one polynucleotide modification enzyme, such as a site-specific recombinase and/or a targeting endonuclease. Site-specific recombinases are well known in the art, and may be generally referred to as invertases, resolvases, or integrases. Non-limiting examples of site-specific recombinases may include lambda integrase, Cre recombinase, FLP recombinase, gamma-delta resolvase, Tn3 resolvase, ΦC31 integrase, Bxb1-integrase, and R4 integrase. Site-specific recombinases recognize specific recognition sequences (or recognition sites) or variants thereof, all of which are well known in the art. For example, Cre recombinases recognize LoxP sites and FLP recombinases recognize FRT sites.

Contemplated targeting endonucleases include zinc finger nucleases (ZFNs), meganucleases, transcription activator-like effector nucleases (TALENs), CRISPR/Cas-like endonucleases, I-Tevl nucleases or related monomeric hybrids, or artificial targeted DNA double strand break inducing agents. Exemplary targeting endonucleases is further described below. For example, typically, a zinc finger nuclease comprises a DNA binding domain (i.e., zinc finger) and a cleavage domain (i.e., nuclease), both of which are described below. Also included in the definition of polynucleotide modification enzymes are any other useful fusion proteins known to those of skill in the art, such as may comprise a DNA binding domain and a nuclease.

A landing pad sequence is a nucleotide sequence comprising at least one recognition sequence that is selectively bound and modified by a specific polynucleotide modification enzyme such as a site-specific recombinase and/or a targeting endonuclease. In general, the recognition sequence(s) in the landing pad sequence does not exist endogenously in the genome of the cell to be modified. For example, where the cell to be modified is a CHO cell, the recognition sequence in the landing pad sequence is not present in the endogenous CHO genome. The rate of targeted integration may be improved by selecting a recognition sequence for a high efficiency nucleotide modifying enzyme that does not exist endogenously within the genome of the targeted cell. Selection of a recognition sequence that does not exist endogenously also reduces potential off-target integration. In other aspects, use of a recognition sequence that is native in the cell to be modified may be desirable. For example, where multiple recognition sequences are employed in the landing pad sequence, one or more may be exogenous, and one or more may be native.

One of ordinary skill in the art can readily determine sequences bound and cut by site-specific recombinases and/or targeting endonucleases.

Another example of a targeting endonuclease that can be used is an RNA-guided endonuclease comprising at least one nuclear localization signal, which permits entry of the endonuclease into the nuclei of eukaryotic cells. The RNA-guided endonuclease also comprises at least one nuclease domain and at least one domain that interacts with a guiding RNA. An RNA-guided endonuclease is directed to a specific chromosomal sequence by a guiding RNA such that the RNA-guided endonuclease cleaves the specific chromosomal sequence. Since the guiding RNA provides the specificity for the targeted cleavage, the endonuclease of the RNA-guided endonuclease is universal and may be used with different guiding RNAs to cleave different target chromosomal sequences. Discussed in further detail below are exemplary RNA-guided endonuclease proteins. For example, the RNA-guided endonuclease can be a CRISPR/Cas protein or a CRISPR/Cas-like fusion protein, an RNA-guided endonuclease derived from a clustered regularly interspersed short palindromic repeats (CRISPR)/CRISPR-associated (Cas) system.

The targeting endonuclease can also be a meganuclease. Meganucleases are endodeoxyribonucleases characterized by a large recognition site, i.e., the recognition site generally ranges from about 12 base pairs to about 40 base pairs. As a consequence of this requirement, the recognition site generally occurs only once in any given genome. Among meganucleases, the family of homing endonucleases named “LAGLIDADG” has become a valuable tool for the study of genomes and genome engineering. Meganucleases may be targeted to specific chromosomal sequence by modifying their recognition sequence using techniques well known to those skilled in the art. See, for example, Epinat et al., 2003, Nuc. Acid Res., 31(11):2952-62 and Stoddard, 2005, Quarterly Review of Biophysics, pp. 1-47.

Yet another example of a targeting endonuclease that can be used is a transcription activator-like effector (TALE) nuclease. TALEs are transcription factors from the plant pathogen Xanthomonas that may be readily engineered to bind new DNA targets. TALEs or truncated versions thereof may be linked to the catalytic domain of endonucleases such as FokI to create targeting endonuclease called TALE nucleases or TALENs. See, e.g., Sanjana et al., 2012, Nature Protocols 7(1):171-192; Bogdanove A J, Voytas D F., 2011, Science, 333(6051):1843-6; Bradley P, Bogdanove A J, Stoddard B L., 2013, Curr Opin Struct Biol., 23(1):93-9.

### V. Methods

Aspects of the current disclosure relate to methods for treating cancer, such as glioblastoma. In further aspects, the therapeutic receptors (e.g., CARs) described herein may be used for stimulating an immune response. The immune response stimulation may be done in vitro, in vivo, or ex vivo. In some aspects, the therapeutic receptors described herein are for preventing relapse. The method generally involves genetically modifying a mammalian cell with an expression vector, or a DNA, an RNA (e.g., in vitro transcribed RNA), or an adeno-associated virus (AAV) comprising nucleotide sequences encoding a polypeptide of the disclosure or directly transferring the polypeptide to the cell. The cell can be an immune cell (e.g., a T lymphocyte or NK cell), a stem cell, a progenitor cell, etc. In some aspects, the cell is a cell described herein.

In some aspects, the genetic modification is carried out ex vivo. For example, a T lymphocyte, a stem cell, or an NK cell (or cell described herein) is obtained from an individual; and the cell obtained from the individual is genetically modified to express a polypeptide of the disclosure. In some cases, the genetically modified cell is activated ex vivo. In other cases, the genetically modified cell is introduced into an individual (e.g., the individual from whom the cell was obtained); and the genetically modified cell is activated in vivo.

### VI. Additional Therapies

A. Immunotherapy

In some aspects, the methods comprise administration of a cancer immunotherapy. Cancer immunotherapy (sometimes called immuno-oncology, abbreviated 10) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies useful in the methods of the disclosure are described below.

1. Checkpoint Inhibitors and Combination Treatment

Aspects of the disclosure may include administration of immune checkpoint inhibitors (also referred to as checkpoint inhibitor therapy), which are further described below. The checkpoint inhibitor therapy may be a monotherapy, targeting only one cellular checkpoint proteins or may be combination therapy that targets at least two cellular checkpoint proteins. For example, the checkpoint inhibitor monotherapy may comprise one of: a PD-1, PD-L1, or PD-L2 inhibitor or may comprise one of a CTLA-4, B7-1, or B7-2 inhibitor. The checkpoint inhibitor combination therapy may comprise one of: a PD-1, PD-L1, or PD-L2 inhibitor and, in combination, may further comprise one of a CTLA-4, B7-1, or B7-2 inhibitor. The combination of inhibitors in combination therapy need not be in the same composition, but can be administered either at the same time, at substantially the same time, or in a dosing regimen that includes periodic administration of both of the inhibitors, wherein the period may be a time period described herein.

a. PD-1, PD-L1, and PD-L2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PD-L1 on epithelial cells and tumor cells. PD-L2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PD-L1 activity.

Alternative names for “PD-1” include CD279 and SLEB2. Alternative names for “PD-L1” include B7-H1, B7-4, CD274, and B7-H. Alternative names for “PD-L2” include B7-DC, Btdc, and CD273. In some aspects, PD-1, PD-L1, and PD-L2 are human PD-1, PD-L1 and PD-L2.

In some aspects, the PD-1 inhibitor is a molecule that inhibits the binding of PD-1 to its ligand binding partners. In a specific aspect, the PD-1 ligand binding partners are PD-L1 and/or PD-L2. In another aspect, a PD-L1 inhibitor is a molecule that inhibits the binding of PD-L1 to its binding partners. In a specific aspect, PD-L1 binding partners are PD-1 and/or B7-1. In another aspect, the PD-L2 inhibitor is a molecule that inhibits the binding of PD-L2 to its binding partners. In a specific aspect, a PD-L2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some aspects, the PD-L1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PD-L2-Fc fusion soluble receptor described in WO2010/027827 and WO2011/066342. Additional PD-1 inhibitors include MEDI0680, also known as AMP-514, and REGN2810.

In some aspects, the immune checkpoint inhibitor is a PD-L1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PD-L2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another aspect, the antibody competes for binding with and/or binds to the same epitope on PD-1, PD-L1, or PD-L2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

b. CTLA-4, B7-1, and B7-2 Inhibitors

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA-4 is a member of the immunoglobulin superfamily that is expressed on the surface of Helper T cells and transmits an inhibitory signal to T cells. CTLA-4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WOO 1/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

2. Inhibition of Co-Stimulatory Molecules

In some aspects, the immunotherapy comprises an inhibitor of a co-stimulatory molecule. In some aspects, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment, they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor.

4. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

5. Adoptive T-Cell Therapy

Adoptive T cell therapy is a form of passive immunization by the transfusion of T-cells (adoptive cell transfer). They are found in blood and tissue and usually activate when they find foreign pathogens. Specifically, they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Tumor targeted T cells can be generated through gene therapy. Tumor targeted T cells can be expanded by exposing the T cells to tumor antigens.

In some aspects, therapeutic cells used in adoptive cell therapies express chimeric antigen receptors (CARs). CARs are fusion proteins that are commonly composed of an extracellular antigen-binding domain (which may be an scFv), an extracellular spacer, a transmembrane domain, costimulatory signaling regions (the number of which varies depending on the specific CAR design), and a CD3-zeta signaling domain/endodomain.

In some aspects, therapeutic cells used in adoptive cell therapies express engineered T-cell receptors (TCRs), which are heterologous TCR molecules that target tumor antigens. Immune cells, including T cells and natural killer (NK) cells, can be engineered to express CARs or TCRs by a variety of methods known in the art, including viral transduction, DNA nucleofection, and RNA nucleofection. Binding of the CAR or TCR to the antigen target can activate human T cells expressing the CAR or TCR, which may result in killing of the cell bearing the antigen or some other immunological response.

In some aspects, the cells comprise a cancer-specific CAR or TCR. The term “cancer-specific” in the context of CAR or TCR polypeptides refers to a polypeptide that has an antigen binding specificity for a cancer-specific molecule, such as a cancer-specific antigen. In some aspects, the cancer-specific CAR and another CAR are on separate polypeptides.

B. Oncolytic Virus

In some aspects, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy.

C. Polysaccharides

In some aspects, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

D. Neoantigens

In some aspects, the additional therapy comprises targeting of neoantigen mutations. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

E. Chemotherapies

In some aspects, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some aspects, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain aspects. In some aspects, the amount of cisplatin delivered to the cell and/or subject in conjunction with the construct comprising an Egr-1 promoter operatively linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain aspects, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable aspect, the chemotherapeutic agent may be administered in an amount effective to cause arrest or regression of the cancer in a host, when the chemotherapy is administered with the construct. In other aspects, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

F. Radiotherapy

In some aspects, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some aspects, the amount of ionizing radiation is greater than 20 Gy and is administered in one dose. In some aspects, the amount of ionizing radiation is 18 Gy and is administered in three doses. In some aspects, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some aspects, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some aspects, the amount of IR may be presented as a total dose of IR, which is then administered in fractionated doses. For example, in some aspects, the total dose is 50 Gy administered in 10 fractionated doses of 5 Gy each. In some aspects, the total dose is 50-90 Gy, administered in 20-60 fractionated doses of 2-3 Gy each. In some aspects, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some aspects, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some aspects, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some aspects, at least, at most, or exactly 1, 2, 3, 4, 5, 6, 7, 8, 9, 10, 11, or 12 (or any derivable range therein) fractionated doses are administered per day. In some aspects, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

G. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

H. Other Agents

It is contemplated that other agents may be used in combination with certain aspects of the present aspects to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other aspects, cytostatic or differentiation agents can be used in combination with certain aspects of the present aspects to improve the anti-hyperproliferative efficacy of the treatments. Inhibitors of cell adhesion are contemplated to improve the efficacy of the present aspects. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present aspects to improve the treatment efficacy.

It is contemplated that a cancer treatment may exclude any of the cancer treatments described herein. Furthermore, aspects of the disclosure include patients that have been previously treated for a therapy described herein, are currently being treated for a therapy described herein, or have not been treated for a therapy described herein. In some aspects, the patient is one that has been determined to be resistant to a therapy described herein. In some aspects, the patient is one that has been determined to be sensitive to a therapy described herein.

### VII. Pharmaceutical Compositions

The present disclosure includes methods for treating disease and modulating immune responses in a subject in need thereof. The disclosure includes cells that may be in the form of a pharmaceutical composition that can be used to induce or modify an immune response.

Administration of the compositions according to the current disclosure will typically be via any common route. This includes, but is not limited to parenteral, orthotopic, intradermal, subcutaneous, orally, transdermally, intratumorally, intramuscular, intraperitoneal, intraperitoneally, intraorbitally, by implantation, by inhalation, intraventricularly, intracerebroventricularly, intranasally, intravenous injection, or into a tumor resection cavity.

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most about or at least about 3, 4, 5, 6, 7, 8, 9, 10 or more. The administrations may range from 2-day to 12-week intervals, more usually from one to two week intervals. The course of the administrations may be followed by assays for alloreactive immune responses and T cell activity.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

The pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (i.e. cells of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

The compositions and related methods of the present disclosure, particularly administration of a composition of the disclosure may also be used in combination with the administration of additional therapies such as the additional therapeutics described herein or in combination with other traditional therapeutics known in the art.

The therapeutic compositions and treatments disclosed herein may precede, be co-current with and/or follow another treatment or agent by intervals ranging from minutes to weeks. In aspects where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

The quantity to be administered, both according to number of treatments and unit dose, depends on the treatment effect desired. An effective dose is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some aspects, the therapeutically effective or sufficient amount of the immune checkpoint inhibitor, such as an antibody and/or microbial modulator, that is administered to a human will be in the range of about 0.01 to about 50 mg/kg of patient body weight whether by one or more administrations. In some aspects, the therapy used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one aspect, a therapy described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain aspects, the effective dose of the pharmaceutical composition is one which can provide a blood level of about 1 μM to 150 μM. In another aspect, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VIII. Therapeutic Methods

The compositions of the disclosure may be used for in vivo, in vitro, or ex vivo administration. The route of administration of the composition may be, for example, intracutaneous, subcutaneous, intravenous, local, topical, and intraperitoneal administrations.

In some aspects, the disclosed methods are directed to methods for treating cancer. The cancer may be a solid tumor, metastatic cancer, or non-metastatic cancer. In certain aspects, the cancer is glioblastoma and may be recurrent, metastatic, relapsed, or of a Stage I, II, III, or IV.

### IX. Sequences

The amino acid sequence of example chimeric polypeptides and CAR molecules useful in the methods and compositions of the present disclosure are provided in Table 1 below.

| | | SEQ |
|---|---|---|
| Description | Sequence | ID NO: |
| SP-IL-13Rα2.BBz | METDTLLLWVLLLWVPGSTGAGGSDYKDDDDKGGSS | 1 |
| CAR; (Murine kappa | PGPVPPSTALRYLIEELVNITQNQKAPLCNGSMVWSINL | |
| signal | TAGMYCAALESLINVSGCSAIEKTQRMLSGFCPHKVSA | |
| sequence_FLAG_SP | GQFSSLHVRDTKIEVAQFVKDLLLHLKKLFREGRFNES | |
| IL13 mutein_IgG4 | KYGPPCPPCPAPEFEGGPSVFLFPPKPKDTLMISRTPEVT | |
| (L235E, | CVVVDVSQEDPEVQFNWYVDGVEVHNAKTKPREEQF | |
| N297Q)_CD28tm_4- | QSTYRVVSVLTVLHQDWLNGKEYKCKVSNKGLPSSIE | |
| 1BB_CD3zeta) | KTISKAKGQPREPQVYTLPPSQEEMTKNQVSLTCLVKG | |
| | FYPSDIAVEWESNGQPENNYKTTPPVLDSDGSFFLYSRL | |
| | TVDKSRWQEGNVFSCSVMHEALHNHYTQKSLSLSLGK | |
| | MFWVLVVVGGVLACYSLLVTVAFIIFWVKRGRKKLLY | |
| | IFKQPFMRPVQTTQEEDGCSCRFPEEEEGGCELRVKFSR | |
| | SADAPAYQQGQNQLYNELNLGRREEYDVLDKRRGRDP | |
| | EMGGKPRRKNPQEGLYNELQKDKMAEAYSEIGMKGE | |
| | RRRGKGHDGLYQGLSTATKDTYDALHMQALPPR | |
| SP-IL-13Rα2.BBz | METDTLLLWVLLLWVSPGSTGSPGPVPPSTALRYLIEEL | 136 |
| CAR; (Murine kappa | VNITQNQKAPLCNGSMVWSINLTAGMYCAALESLINVS | |
| signal sequence_SP | GCSAIEKTQRMLSGFCPHKVSAGQFSSLHVRDTKIEVA | |
| IL13 mutein_IgG4 | QFVKDLLLHLKKLFREGRFNESKYGPPCPPCPAPEFEGG | |
| (L235E, | PSVFLFPPKPKDTLMISRTPEVTCVVVDVSQEDPEVQFN | |
| N297Q)_CD28tm_4- | WYVDGVEVHNAKTKPREEQFQSTYRVVSVLTVLHQD | |
| 1BB_CD3zeta) | WLNGKEYKCKVSNKGLPSSIEKTISKAKGQPREPQVYT | |
| | LPPSQEEMTKNQVSLTCLVKGFYPSDIAVEWESNGQPE | |
| | NNYKTTPPVLDSDGSFFLYSRLTVDKSRWQEGNVFSCS | |
| | VMHEALHNHYTQKSLSLSLGKMFWVLVVVGGVLACY | |
| | SLLVTVAFIIFWVKRGRKKLLYIFKQPFMRPVQTTQEED | |
| | GCSCRFPEEEEGGCELRVKFSRSADAPAYQQGQNQLYN | |
| | ELNLGRREEYDVLDKRRGRDPEMGGKPRRKNPQEGLY | |
| | NELQKDKMAEAYSEIGMKGERRRGKGHDGLYQGLST | |
| | ATKDTYDALHMQALPPR | |
| MK signal sequence | METDTLLLWVLLLWVPGSTG | 2 |
| FLAG | DYKDDDDK | 3 |
| SP IL13 mutein | SPGPVPPSTALRYLIEELVNITQNQKAPLCNGSMVWSIN | 4 |
| | LTAGMYCAALESLINVSGCSAIEKTORMLSGFCPHKVS | |
| | AGQFSSLHVRDTKIEVAQFVKDLLLHLKKLFREGRFN | |
| IgG4 (L235E, N297Q) | ESKYGPPCPPCPAPEFEGGPSVFLFPPKPKDTLMISRTPE | 5 |
| | VTCVVVDVSQEDPEVQFNWYVDGVEVHNAKTKPREE | |
| | QFQSTYRVVSVLTVLHQDWLNGKEYKCKVSNKGLPSS | |
| | IEKTISKAKGQPREPQVYTLPPSQEEMTKNQVSLTCLVK | |
| | GFYPSDIAVEWESNGQPENNYKTTPPVLDSDGSFFLYSR | |
| | LTVDKSRWQEGNVFSCSVMHEALHNHYTQKSLSLSLG | |
| | K | |
| CD28 transmembrane | MFWVLVVVGGVLACYSLLVTVAFIIFWV | 6 |
| domain | | |
| 4-1BB co-stimulatory | KRGRKKLLYIFKQPFMRPVQTTQEEDGCSCRFPEEEEG | 7 |
| | GCEL | |
| CD3 Zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| SP-IL-13Rα2/TGF- | METDTLLLWVLLLWVPGSTGAGGSDYKDDDDKGGSS | 9 |
| βBBz CAR; (Murine | PGPVPPSTALRYLIEELVNITQNQKAPLCNGSMVWSINL | |
| kappa signal | TAGMYCAALESLINVSGCSAIEKTQRMLSGFCPHKVSA | |
| sequence_FLAG_SP | GQFSSLHVRDTKIEVAQFVKDLLLHLKKLFREGRFNGG | |
| IL13 | GGSGGGGSGGGGSGGGGSQVQLVQSGAEVKKPGSSVK | |
| mutein_(G4S)x4_TGF- | VSCKASGYTFSSNVISWVRQAPGQGLEWMGGVIPIVDI | |
| β scFv_IgG4 | ANYAQRFKGRVTITADESTSTTYMELSSLRSEDTAVYY | |
| hinge_CD28tm_4- | CALPRAFVLDAMDYWGQGTLVTVSSGGGGSGGGGSG | |
| 1BB_CD3zeta) | GGGSETVLTQSPGTLSLSPGERATLSCRASQSLGSSYLA | |
| | WYQQKPGQAPRLLIYGASSRAPGIPDRFSGSGSGTDFTL | |
| | TISRLEPEDFAVYYCQQYADSPITFGQGTRLEIKESKYG | |
| | PPCPPCPMFWVLVVVGGVLACYSLLVTVAFIIFWVKRG | |
| | RKKLLYIFKQPFMRPVQTTQEEDGCSCRFPEEEEGGCEL | |
| | RVKFSRSADAPAYQQGQNQLYNELNLGRREEYDVLDK | |
| | RRGRDPEMGGKPRRKNPQEGLYNELQKDKMAEAYSEI | |
| | GMKGERRRGKGHDGLYQGLSTATKDTYDALHMQALP | |
| | PR | |
| SP-IL-13Rα2/TGF- | METDTLLLWVLLLWVPGSTGSPGPVPPSTALRYLIEEL | 137 |
| βBBz CAR; (Murine | VNITQNQKAPLCNGSMVWSINLTAGMYCAALESLINVS | |
| kappa signal sequence_ | GCSAIEKTQRMLSGFCPHKVSAGQFSSLHVRDTKIEVA | |
| SP IL13 | QFVKDLLLHLKKLFREGRFNGGGGSGGGGSGGGGSGG | |
| mutein_(G4S)x4_TGF- | GGSQVQLVQSGAEVKKPGSSVKVSCKASGYTFSSNVIS | |
| β scFv_IgG4 | WVRQAPGQGLEWMGGVIPIVDIANYAQRFKGRVTITA | |
| hinge_CD28tm_4- | DESTSTTYMELSSLRSEDTAVYYCALPRAFVLDAMDY | |
| 1BB_CD3zeta) | WGQGTLVTVSSGGGGSGGGGSGGGGSETVLTQSPGTL | |
| | SLSPGERATLSCRASQSLGSSYLAWYQQKPGQAPRLLI | |
| | YGASSRAPGIPDRFSGSGSGTDFTLTISRLEPEDFAVYYC | |
| | QQYADSPITFGQGTRLEIKESKYGPPCPPCPMFWVLVV | |
| | VGGVLACYSLLVTVAFIIFWVKRGRKKLLYIFKQPFMR | |
| | PVQTTQEEDGCSCRFPEEEEGGCELRVKFSRSADAPAY | |
| | QQGQNQLYNELNLGRREEYDVLDKRRGRDPEMGGKP | |
| | RRKNPQEGLYNELQKDKMAEAYSEIGMKGERRRGKG | |
| | HDGLYQGLSTATKDTYDALHMQALPPR | |
| MK signal sequence | METDTLLLWVLLLWVPGSTG | 2 |
| FLAG | DYKDDDDK | 3 |
| SP IL13 mutein | SPGPVPPSTALRYLIEELVNITQNQKAPLCNGSMVWSIN | 4 |
| | LTAGMYCAALESLINVSGCSAIEKTQRMLSGFCPHKVS | |
| | AGQFSSLHVRDTKIEVAQFVKDLLLHLKKLFREGRFN | |
| (G4S)x4 | GGGGSGGGGSGGGGSGGGGS | 10 |
| TGF-β scFv | QVQLVQSGAEVKKPGSSVKVSCKASGYTFSSNVISWVR | 11 |
| | QAPGQGLEWMGGVIPIVDIANYAQRFKGRVTITADEST | |
| | STTYMELSSLRSEDTAVYYCALPRAFVLDAMDYWGQG | |
| | TLVTVSSGGGGSGGGGSGGGGSETVLTQSPGTLSLSPG | |
| | ERATLSCRASQSLGSSYLAWYQQKPGQAPRLLIYGASS | |
| | RAPGIPDRFSGSGSGTDFTLTISRLEPEDFAVYYCQQYA | |
| | DSPITFGQGTRLEIK | |
| IgG4 hinge | ESKYGPPCPPCP | 12 |
| CD28 transmembrane | MFWVLVVVGGVLACYSLLVTVAFIIFWV | 6 |
| domain | | |
| 4-1BB co-stimulatory | KRGRKKLLYIFKQPFMRPVQTTQEEDGCSCRFPEEEEG | 7 |
| | GCEL | |
| CD3 Zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| SP-IL-13Rα2.BBz | METDTLLLWVLLLWVPGSTGAGGSDYKDDDDKGGSS | 13 |
| (KR) CAR; (Murine | PGPVPPSTALRYLIEELVNITQNQKAPLCNGSMVWSINL | |
| kappa signal | TAGMYCAALESLINVSGCSAIEKTQRMLSGFCPHKVSA | |
| sequence_FLAG_SP | GQFSSLHVRDTKIEVAQFVKDLLLHLKKLFREGRFNES | |
| IL13 mutein_IgG4 | KYGPPCPPCPAPEFEGGPSVFLFPPKPKDTLMISRTPEVT | |
| (L235E, | CVVVDVSQEDPEVQFNWYVDGVEVHNAKTKPREEQF | |
| N297Q)_CD28tm_4- | QSTYRVVSVLTVLHQDWLNGKEYKCKVSNKGLPSSIE | |
| 1BB(KR)_CD3zeta(KR | KTISKAKGQPREPQVYTLPPSQEEMTKNQVSLTCLVKG | |
| | FYPSDIAVEWESNGQPENNYKTTPPVLDSDGSFFLYSRL | |
| | TVDKSRWQEGNVFSCSVMHEALHNHYTQKSLSLSLGK | |
| | MFWVLVVVGGVLACYSLLVTVAFIIFWVRRGRRRLLYI | |
| | FRQPFMRPVQTTQEEDGCSCRFPEEEEGGCELRVRFSRS | |
| | ADAPAYQQGQNQLYNELNLGRREEYDVLDRRRGRDPE | |
| | MGGRPRRRNPQEGLYNELQRDRMAEAYSEIGMRGERR | |
| | RGRGHDGLYQGLSTATRDTYDALHMQALPPR | |
| SP-IL-13Rα2.BBz | METDTLLLWVLLLWVPGSTGSPGPVPPSTALRYLIEEL | 138 |
| (KR) CAR; (Murine | VNITQNQKAPLCNGSMVWSINLTAGMYCAALESLINVS | |
| kappa signal sequence_ | GCSAIEKTQRMLSGFCPHKVSAGQFSSLHVRDTKIEVA | |
| SP IL13 mutein_IgG4 | QFVKDLLLHLKKLFREGRFNESKYGPPCPPCPAPEFEGG | |
| (L235E, | PSVFLFPPKPKDTLMISRTPEVTCVVVDVSQEDPEVQFN | |
| N297Q)_CD28tm_4- | WYVDGVEVHNAKTKPREEQFQSTYRVVSVLTVLHQD | |
| 1BB(KR)_CD3zeta(KR | WLNGKEYKCKVSNKGLPSSIEKTISKAKGQPREPQVYT | |
| | LPPSQEEMTKNQVSLTCLVKGFYPSDIAVEWESNGQPE | |
| | NNYKTTPPVLDSDGSFFLYSRLTVDKSRWQEGNVFSCS | |
| | VMHEALHNHYTQKSLSLSLGKMFWVLVVVGGVLACY | |
| | SLLVTVAFIIFWVRRGRRRLLYIFRQPFMRPVQTTQEED | |
| | GCSCRFPEEEEGGCELRVRFSRSADAPAYQQGQNQLYN | |
| | ELNLGRREEYDVLDRRRGRDPEMGGRPRRRNPQEGLY | |
| | NELQRDRMAEAYSEIGMRGERRRGRGHDGLYQGLSTA | |
| | TRDTYDALHMQALPPR | |
| MK signal sequence | METDTLLLWVLLLWVPGSTG | 2 |
| FLAG | DYKDDDDK | 3 |
| SP IL13 mutein | SPGPVPPSTALRYLIEELVNITQNQKAPLCNGSMVWSIN | 4 |
| | LTAGMYCAALESLINVSGCSAIEKTQRMLSGFCPHKVS | |
| | AGQFSSLHVRDTKIEVAQFVKDLLLHLKKLFREGRFN | |
| IgG4 (L235E, N297Q) | ESKYGPPCPPCPAPEFEGGPSVFLFPPKPKDTLMISRTPE | 5 |
| | VTCVVVDVSQEDPEVQFNWYVDGVEVHNAKTKPREE | |
| | QFQSTYRVVSVLTVLHQDWLNGKEYKCKVSNKGLPSS | |
| | IEKTISKAKGQPREPQVYTLPPSQEEMTKNQVSLTCLVK | |
| | GFYPSDIAVEWESNGQPENNYKTTPPVLDSDGSFFLYSR | |
| | LTVDKSRWQEGNVFSCSVMHEALHNHYTQKSLSLSLG | |
| | K | |
| CD28 transmembrane | MFWVLVVVGGVLACYSLLVTVAFIIFWV | 6 |
| domain | | |
| 4-1BB(KR) co- | RRGRRRLLYIFRQPFMRPVQTTQEEDGCSCRFPEEEEGG | 14 |
| stimulatory | CEL | |
| CD3 Zeta(KR) | RVRFSRSADAPAYQQGQNQLYNELNLGRREEYDVLDR | 15 |
| | RRGRDPEMGGRPRRRNPQEGLYNELQRDRMAEAYSEI | |
| | GMRGERRRGRGHDGLYQGLSTATRDTYDALHMQALP | |
| | PR | |
| SP-IL-13Rα2/TGF- | METDTLLLWVLLLWVPGSTGAGGSDYKDDDDKGGSS | 16 |
| βBBZ (KR) CAR; | PGPVPPSTALRYLIEELVNITQNQKAPLCNGSMVWSINL | |
| (Murine kappa signal | TAGMYCAALESLINVSGCSAIEKTQRMLSGFCPHKVSA | |
| sequence_FLAG_SP | GQFSSLHVRDTKIEVAQFVKDLLLHLKKLFREGRFNGG | |
| IL13 | GGSGGGGSGGGGSGGGGSQVQLVQSGAEVKKPGSSVK | |
| mutein_(G4S)x4_TGF- | VSCKASGYTFSSNVISWVRQAPGQGLEWMGGVIPIVDI | |
| β scFv_IgG4 | ANYAQRFKGRVTITADESTSTTYMELSSLRSEDTAVYY | |
| hinge_CD28tm_4- | CALPRAFVLDAMDYWGQGTLVTVSSGGGGSGGGGSG | |
| 1BB(KR)_CD3zeta | GGGSETVLTQSPGTLSLSPGERATLSCRASQSLGSSYLA | |
| (KR)) | WYQQKPGQAPRLLIYGASSRAPGIPDRFSGSGSGTDFTL | |
| | TISRLEPEDFAVYYCQQYADSPITFGQGTRLEIKESKYG | |
| | PPCPPCPMFWVLVVVGGVLACYSLLVTVAFIIFWVRRG | |
| | RRRLLYIFRQPFMRPVQTTQEEDGCSCRFPEEEEGGCEL | |
| | RVRFSRSADAPAYQQGQNQLYNELNLGRREEYDVLDR | |
| | RRGRDPEMGGRPRRRNPQEGLYNELQRDRMAEAYSEI | |
| | GMRGERRRGRGHDGLYQGLSTATRDTYDALHMQALP | |
| | PR | |
| SP-IL-13Rα2/TGF- | METDTLLLWVLLLWVPGSTGSPGPVPPSTALRYLIEEL | 139 |
| βBBZ (KR) CAR; | VNITQNQKAPLCNGSMVWSINLTAGMYCAALESLINVS | |
| (Murine kappa signal | GCSAIEKTQRMLSGFCPHKVSAGQFSSLHVRDTKIEVA | |
| sequence_SP IL13 | QFVKDLLLHLKKLFREGRFNGGGGSGGGGSGGGGSGG | |
| mutein_(G4S)x4_TGF- | GGSQVQLVQSGAEVKKPGSSVKVSCKASGYTFSSNVIS | |
| β scFv_IgG4 | WVRQAPGQGLEWMGGVIPIVDIANYAQRFKGRVTITA | |
| hinge_CD28tm_4- | DESTSTTYMELSSLRSEDTAVYYCALPRAFVLDAMDY | |
| 1BB(KR)_CD3zeta | WGQGTLVTVSSGGGGSGGGGSGGGGSETVLTQSPGTL | |
| (KR)) | SLSPGERATLSCRASQSLGSSYLAWYQQKPGQAPRLLI | |
| | YGASSRAPGIPDRFSGSGSGTDFTLTISRLEPEDFAVYYC | |
| | QQYADSPITFGQGTRLEIKESKYGPPCPPCPMFWVLVV | |
| | VGGVLACYSLLVTVAFIIFWVRRGRRRLLYIFRQPFMRP | |
| | VQTTQEEDGCSCRFPEEEEGGCELRVRFSRSADAPAYQ | |
| | QGQNQLYNELNLGRREEYDVLDRRRGRDPEMGGRPRR | |
| | RNPQEGLYNELQRDRMAEAYSEIGMRGERRRGRGHDG | |
| | LYQGLSTATRDTYDALHMQALPPR | |
| MK signal sequence | METDTLLLWVLLLWVPGSTG | 2 |
| FLAG | DYKDDDDK | 3 |
| SP IL13 mutein | SPGPVPPSTALRYLIEELVNITQNQKAPLCNGSMVWSIN | 4 |
| | LTAGMYCAALESLINVSGCSAIEKTQRMLSGFCPHKVS | |
| | AGQFSSLHVRDTKIEVAQFVKDLLLHLKKLFREGRFN | |
| (G4S)×4 | GGGGSGGGGSGGGGSGGGGS | 10 |
| TGF-β scFv | QVQLVQSGAEVKKPGSSVKVSCKASGYTFSSNVISWVR | 11 |
| | QAPGQGLEWMGGVIPIVDIANYAQRFKGRVTITADEST | |
| | STTYMELSSLRSEDTAVYYCALPRAFVLDAMDYWGQG | |
| | TLVTVSSGGGGSGGGGSGGGGSETVLTQSPGTLSLSPG | |
| | ERATLSCRASQSLGSSYLAWYQQKPGQAPRLLIYGASS | |
| | RAPGIPDRFSGSGSGTDFTLTISRLEPEDFAVYYCQQYA | |
| | DSPITFGQGTRLEIK | |
| IgG4 hinge | ESKYGPPCPPCP | 12 |
| CD28 transmembrane | MFWVLVVVGGVLACYSLLVTVAFIIFWV | 6 |
| domain | | |
| 4-1BB(KR) co- | RRGRRRLLYIFRQPFMRPVQTTQEEDGCSCRFPEEEEGG | 14 |
| stimulatory | CEL | |
| CD3zeta(KR) | RVRFSRSADAPAYQQGQNQLYNELNLGRREEYDVLDR | 15 |
| | RRGRDPEMGGRPRRRNPQEGLYNELQRDRMAEAYSEI | |
| | GMRGERRRGRGHDGLYQGLSTATRDTYDALHMQALP | |
| | PR | |
| SP-IL-13Rα2.28z | METDTLLLWVLLLWVPGSTGAGGSDYKDDDDKGGSS | 17 |
| CAR; (Murine kappa | PGPVPPSTALRYLIEELVNITQNQKAPLCNGSMVWSINL | |
| signal | TAGMYCAALESLINVSGCSAIEKTQRMLSGFCPHKVSA | |
| sequence_FLAG_SP | GQFSSLHVRDTKIEVAQFVKDLLLHLKKLFREGRFNES | |
| IL13 mutein_IgG4 | KYGPPCPPCPAPEFEGGPSVFLFPPKPKDTLMISRTPEVT | |
| (L235E, | CVVVDVSQEDPEVQFNWYVDGVEVHNAKTKPREEQF | |
| N297Q)_CD28tm_CD2 | QSTYRVVSVLTVLHQDWLNGKEYKCKVSNKGLPSSIE | |
| 8cyto_CD3zeta) | KTISKAKGQPREPQVYTLPPSQEEMTKNQVSLTCLVKG | |
| | FYPSDIAVEWESNGQPENNYKTTPPVLDSDGSFFLYSRL | |
| | TVDKSRWQEGNVFSCSVMHEALHNHYTQKSLSLSLGK | |
| | MFWVLVVVGGVLACYSLLVTVAFIIFWVRSKRSRGGH | |
| | SDYMNMTPRRPGPTRKHYQPYAPPRDFAAYRSGGGRV | |
| | KFSRSADAPAYQQGQNQLYNELNLGRREEYDVLDKRR | |
| | GRDPEMGGKPRRKNPQEGLYNELQKDKMAEAYSEIG | |
| | MKGERRRGKGHDGLYQGLSTATKDTYDALHMQALPP | |
| | R | |
| SP-IL-13Rα2.28z | METDTLLLWVLLLWVPGSTGSPGPVPPSTALRYLIEEL | 140 |
| CAR; (Murine kappa | VNITQNQKAPLCNGSMVWSINLTAGMYCAALESLINVS | |
| signal sequence_ SP | GCSAIEKTQRMLSGFCPHKVSAGQFSSLHVRDTKIEVA | |
| IL13 mutein_IgG4 | QFVKDLLLHLKKLFREGRFNESKYGPPCPPCPAPEFEGG | |
| (L235E, | PSVFLFPPKPKDTLMISRTPEVTCVVVDVSQEDPEVQFN | |
| N297Q)_CD28tm_CD2 | WYVDGVEVHNAKTKPREEQFQSTYRVVSVLTVLHQD | |
| 8cyto_CD3zeta) | WLNGKEYKCKVSNKGLPSSIEKTISKAKGQPREPQVYT | |
| | LPPSQEEMTKNQVSLTCLVKGFYPSDIAVEWESNGQPE | |
| | NNYKTTPPVLDSDGSFFLYSRLTVDKSRWQEGNVFSCS | |
| | VMHEALHNHYTQKSLSLSLGKMFWVLVVVGGVLACY | |
| | SLLVTVAFIIFWVRSKRSRGGHSDYMNMTPRRPGPTRK | |
| | HYQPYAPPRDFAAYRSGGGRVKFSRSADAPAYQQGQN | |
| | QLYNELNLGRREEYDVLDKRRGRDPEMGGKPRRKNPQ | |
| | EGLYNELQKDKMAEAYSEIGMKGERRRGKGHDGLYQ | |
| | GLSTATKDTYDALHMQALPPR | |
| MK signal sequence | METDTLLLWVLLLWVPGSTG | 2 |
| FLAG | DYKDDDDK | 3 |
| SP IL13 mutein | SPGPVPPSTALRYLIEELVNITQNQKAPLCNGSMVWSIN | 4 |
| | LTAGMYCAALESLINVSGCSAIEKTORMLSGFCPHKVS | |
| | AGQFSSLHVRDTKIEVAQFVKDLLLHLKKLFREGRFN | |
| IgG4 (L235E, N297Q) | ESKYGPPCPPCPAPEFEGGPSVFLFPPKPKDTLMISRTPE | 5 |
| | VTCVVVDVSQEDPEVQFNWYVDGVEVHNAKTKPREE | |
| | QFQSTYRVVSVLTVLHQDWLNGKEYKCKVSNKGLPSS | |
| | IEKTISKAKGQPREPQVYTLPPSQEEMTKNQVSLTCLVK | |
| | GFYPSDIAVEWESNGQPENNYKTTPPVLDSDGSFFLYSR | |
| | LTVDKSRWQEGNVFSCSVMHEALHNHYTQKSLSLSLG | |
| | K | |
| CD28tm | MFWVLVVVGGVLACYSLLVTVAFIIFWV | 6 |
| CD28cyto | RSKRSRGGHSDYMNMTPRRPGPTRKHYQPYAPPRDFA | 18 |
| | AYRS | |
| CD3zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| SP-IL-13Rα2/TGF- | METDTLLLWVLLLWVPGSTGAGGSDYKDDDDKGGSS | 25 |
| β.28z CAR; (Murine | PGPVPPSTALRYLIEELVNITQNQKAPLCNGSMVWSINL | |
| kappa signal | TAGMYCAALESLINVSGCSAIEKTQRMLSGFCPHKVSA | |
| sequence_FLAG_SP | GQFSSLHVRDTKIEVAQFVKDLLLHLKKLFREGRFNGG | |
| IL13 | GGSGGGGSGGGGSGGGGSQVQLVQSGAEVKKPGSSVK | |
| mutein_(G4S)x4_TGF- | VSCKASGYTFSSNVISWVRQAPGQGLEWMGGVIPIVDI | |
| β scFv_IgG4 | ANYAQRFKGRVTITADESTSTTYMELSSLRSEDTAVYY | |
| hinge_CD28tm_ | CALPRAFVLDAMDYWGQGTLVTVSSGGGGSGGGGSG | |
| CD28cyto_CD3zeta) | GGGSETVLTQSPGTLSLSPGERATLSCRASQSLGSSYLA | |
| | WYQQKPGQAPRLLIYGASSRAPGIPDRFSGSGSGTDFTL | |
| | TISRLEPEDFAVYYCQQYADSPITFGQGTRLEIKESKYG | |
| | PPCPPCPMFWVLVVVGGVLACYSLLVTVAFIIFWVRSK | |
| | RSRGGHSDYMNMTPRRPGPTRKHYQPYAPPRDFAAYR | |
| | SGGGRVKFSRSADAPAYQQGQNQLYNELNLGRREEYD | |
| | VLDKRRGRDPEMGGKPRRKNPQEGLYNELQKDKMAE | |
| | AYSEIGMKGERRRGKGHDGLYQGLSTATKDTYDALH | |
| | MQALPPR | |
| SP-IL-13Rα2/TGF- | METDTLLLWVLLLWVPGSTGSPGPVPPSTALRYLIEEL | 141 |
| β.28z CAR; (Murine | VNITQNQKAPLCNGSMVWSINLTAGMYCAALESLINVS | |
| kappa signal sequence_ | GCSAIEKTQRMLSGFCPHKVSAGQFSSLHVRDTKIEVA | |
| SP IL13 | QFVKDLLLHLKKLFREGRFNGGGGSGGGGSGGGGSGG | |
| mutein_(G4S)x4_TGF- | GGSQVQLVQSGAEVKKPGSSVKVSCKASGYTFSSNVIS | |
| β scFv_IgG4 | WVRQAPGQGLEWMGGVIPIVDIANYAQRFKGRVTITA | |
| hinge_CD28tm | DESTSTTYMELSSLRSEDTAVYYCALPRAFVLDAMDY | |
| CD28cyto_CD3zeta | WGQGTLVTVSSGGGGSGGGGSGGGGSETVLTQSPGTL | |
| | SLSPGERATLSCRASQSLGSSYLAWYQQKPGQAPRLLI | |
| | YGASSRAPGIPDRFSGSGSGTDFTLTISRLEPEDFAVYYC | |
| | QQYADSPITFGQGTRLEIKESKYGPPCPPCPMFWVLVV | |
| | VGGVLACYSLLVTVAFIIFWVRSKRSRGGHSDYMNMT | |
| | PRRPGPTRKHYQPYAPPRDFAAYRSGGGRVKFSRSADA | |
| | PAYQQGQNQLYNELNLGRREEYDVLDKRRGRDPEMG | |
| | GKPRRKNPQEGLYNELQKDKMAEAYSEIGMKGERRRG | |
| | KGHDGLYQGLSTATKDTYDALHMQALPPR | |
| MK signal sequence | METDTLLLWVLLLWVPGST | 2 |
| FLAG | DYKDDDDK | 3 |
| SP IL13 mutein | SPGPVPPSTALRYLIEELVNITQNQKAPLCNGSMVWSIN | 4 |
| | LTAGMYCAALESLINVSGCSAIEKTQRMLSGFCPHKVS | |
| | AGQFSSLHVRDTKIEVAQFVKDLLLHLKKLFREGRFN | |
| (G4S)x4 | GGGGSGGGGSGGGGSGGGGS | 10 |
| TGF-β scFv | QVQLVQSGAEVKKPGSSVKVSCKASGYTFSSNVISWVR | 11 |
| | QAPGQGLEWMGGVIPIVDIANYAQRFKGRVTITADEST | |
| | STTYMELSSLRSEDTAVYYCALPRAFVLDAMDYWGQG | |
| | TLVTVSSGGGGSGGGGSGGGGSETVLTQSPGTLSLSPG | |
| | ERATLSCRASQSLGSSYLAWYQQKPGQAPRLLIYGASS | |
| | RAPGIPDRFSGSGSGTDFTLTISRLEPEDFAVYYCQQYA | |
| | DSPITFGQGTRLEIK | |
| IgG4 hinge | ESKYGPPCPPCP | 12 |
| CD28tm | MFWVLVVVGGVLACYSLLVTVAFIIFWV | 6 |
| CD28cyto | RSKRSRGGHSDYMNMTPRRPGPTRKHYQPYAPPRDFA | 18 |
| | AYRS | |
| CD3zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| Full IL-13Rα2.BBz | METDTLLLWVLLLWVPGSTGAGGSDYKDDDDKGGSL | 19 |
| CAR; (Murine kappa | TCLGGFASPGPVPPSTALRYLIEELVNITQNQKAPLCNG | |
| signal | SMVWSINLTAGMYCAALESLINVSGCSAIEKTQRMLSG | |
| sequence_FLAG_Full | FCPHKVSAGQFSSLHVRDTKIEVAQFVKDLLLHLKKLF | |
| IL13 mutein_IgG4 | REGRFNESKYGPPCPPCPAPEFEGGPSVFLFPPKPKDTL | |
| (L235E, | MISRTPEVTCVVVDVSQEDPEVQFNWYVDGVEVHNAK | |
| N297Q)_CD28tm_4- | TKPREEQFQSTYRVVSVLTVLHQDWLNGKEYKCKVSN | |
| 1BB_CD3zeta) | KGLPSSIEKTISKAKGQPREPQVYTLPPSQEEMTKNQVS | |
| | LTCLVKGFYPSDIAVEWESNGQPENNYKTTPPVLDSDG | |
| | SFFLYSRLTVDKSRWQEGNVFSCSVMHEALHNHYTQK | |
| | SLSLSLGKMFWVLVVVGGVLACYSLLVTVAFIIFWVKR | |
| | GRKKLLYIFKQPFMRPVQTTQEEDGCSCRFPEEEEGGC | |
| | ELRVKFSRSADAPAYQQGQNQLYNELNLGRREEYDVL | |
| | DKRRGRDPEMGGKPRRKNPQEGLYNELQKDKMAEAY | |
| | SEIGMKGERRRGKGHDGLYQGLSTATKDTYDALHMQ | |
| | ALPPR | |
| Full IL-13Rα2.BBz | METDTLLLWVLLLWVPGSTGLTCLGGFASPGPVPPSTA | 142 |
| CAR; (Murine kappa | LRYLIEELVNITQNQKAPLCNGSMVWSINLTAGMYCAA | |
| signal sequence_Full | LESLINVSGCSAIEKTQRMLSGFCPHKVSAGQFSSLHVR | |
| IL13 mutein_IgG4 | DTKIEVAQFVKDLLLHLKKLFREGRFNESKYGPPCPPCP | |
| (L235E, | APEFEGGPSVFLFPPKPKDTLMISRTPEVTCVVVDVSQE | |
| N297Q)_CD28tm_4- | DPEVQFNWYVDGVEVHNAKTKPREEQFQSTYRVVSVL | |
| 1BB_CD3zeta) | TVLHQDWLNGKEYKCKVSNKGLPSSIEKTISKAKGQPR | |
| | EPQVYTLPPSQEEMTKNQVSLTCLVKGFYPSDIAVEWE | |
| | SNGQPENNYKTTPPVLDSDGSFFLYSRLTVDKSRWQEG | |
| | NVFSCSVMHEALHNHYTQKSLSLSLGKMFWVLVVVG | |
| | GVLACYSLLVTVAFIIFWVKRGRKKLLYIFKQPFMRPV | |
| | QTTQEEDGCSCRFPEEEEGGCELRVKFSRSADAPAYQQ | |
| | GQNQLYNELNLGRREEYDVLDKRRGRDPEMGGKPRR | |
| | KNPQEGLYNELQKDKMAEAYSEIGMKGERRRGKGHD | |
| | GLYQGLSTATKDTYDALHMQALPPR | |
| MK signal sequence | METDTLLLWVLLLWVPGSTG | 2 |
| FLAG | DYKDDDDK | 3 |
| Full IL13 mutein | LTCLGGFASPGPVPPSTALRYLIEELVNITQNQKAPLCN | 20 |
| | GSMVWSINLTAGMYCAALESLINVSGCSAIEKTQRMLS | |
| | GFCPHKVSAGQFSSLHVRDTKIEVAQFVKDLLLHLKKL | |
| | FREGREN | |
| IgG4 (L235E, N297Q) | ESKYGPPCPPCPAPEFEGGPSVFLFPPKPKDTLMISRTPE | 5 |
| | VTCVVVDVSQEDPEVQFNWYVDGVEVHNAKTKPREE | |
| | QFQSTYRVVSVLTVLHQDWLNGKEYKCKVSNKGLPSS | |
| | IEKTISKAKGQPREPQVYTLPPSQEEMTKNQVSLTCLVK | |
| | GFYPSDIAVEWESNGQPENNYKTTPPVLDSDGSFFLYSR | |
| | LTVDKSRWQEGNVFSCSVMHEALHNHYTQKSLSLSLG | |
| | K | |
| CD28tm | MFWVLVVVGGVLACYSLLVTVAFIIFWV | 6 |
| 4-1BB | KRGRKKLLYIFKQPFMRPVQTTQEEDGCSCRFPEEEEG | 7 |
| | GCEL | |
| CD3zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| Full-IL-13Rα2/TGF- | METDTLLLWVLLLWVPGSTGAGGSDYKDDDDKGGSL | 21 |
| βBBz CAR; (Murine | TCLGGFASPGPVPPSTALRYLIEELVNITQNQKAPLCNG | |
| kappa signal | SMVWSINLTAGMYCAALESLINVSGCSAIEKTQRMLSG | |
| sequence_FLAG_Full | FCPHKVSAGQFSSLHVRDTKIEVAQFVKDLLLHLKKLF | |
| IL13 | REGRFNGGGGSGGGGSGGGGSGGGGSQVQLVQSGAE | |
| mutein_(G4S)x4_TGF- | VKKPGSSVKVSCKASGYTFSSNVISWVRQAPGQGLEW | |
| β scFv_IgG4 | MGGVIPIVDIANYAQRFKGRVTITADESTSTTYMELSSL | |
| hinge_CD28tm_4- | RSEDTAVYYCALPRAFVLDAMDYWGQGTLVTVSSGG | |
| 1BB_CD3zeta) | GGSGGGGSGGGGSETVLTQSPGTLSLSPGERATLSCRA | |
| | SQSLGSSYLAWYQQKPGQAPRLLIYGASSRAPGIPDRFS | |
| | GSGSGTDFTLTISRLEPEDFAVYYCQQYADSPITFGQGT | |
| | RLEIKESKYGPPCPPCPMFWVLVVVGGVLACYSLLVTV | |
| | AFIIFWVKRGRKKLLYIFKQPFMRPVQTTQEEDGCSCRF | |
| | PEEEEGGCELRVKFSRSADAPAYQQGQNQLYNELNLG | |
| | RREEYDVLDKRRGRDPEMGGKPRRKNPQEGLYNELQK | |
| | DKMAEAYSEIGMKGERRRGKGHDGLYQGLSTATKDT | |
| | YDALHMQALPPR | |
| Full IL-13Rα2/TGF- | METDTLLLWVLLLWVPGSTGLTCLGGFASPGPVPPSTA | 143 |
| βBBz CAR; (Murine | LRYLIEELVNITQNQKAPLCNGSMVWSINLTAGMYCAA | |
| kappa signal sequence | LESLINVSGCSAIEKTQRMLSGFCPHKVSAGQFSSLHVR | |
| Full IL13 | DTKIEVAQFVKDLLLHLKKLFREGRFNGGGGSGGGGS | |
| mutein_(G4S)x4_TGF- | GGGGSGGGGSQVQLVQSGAEVKKPGSSVKVSCKASGY | |
| β scFv_IgG4 | TFSSNVISWVRQAPGQGLEWMGGVIPIVDIANYAQRFK | |
| hinge_CD28tm_4- | GRVTITADESTSTTYMELSSLRSEDTAVYYCALPRAFVL | |
| 1BB_CD3zeta) | DAMDYWGQGTLVTVSSGGGGSGGGGSGGGGSETVLT | |
| | QSPGTLSLSPGERATLSCRASQSLGSSYLAWYQQKPGQ | |
| | APRLLIYGASSRAPGIPDRFSGSGSGTDFTLTISRLEPEDF | |
| | AVYYCQQYADSPITFGQGTRLEIKESKYGPPCPPCPMF | |
| | WVLVVVGGVLACYSLLVTVAFIIFWVKRGRKKLLYIFK | |
| | QPFMRPVQTTQEEDGCSCRFPEEEEGGCELRVKFSRSA | |
| | DAPAYQQGQNQLYNELNLGRREEYDVLDKRRGRDPE | |
| | MGGKPRRKNPQEGLYNELQKDKMAEAYSEIGMKGER | |
| | RRGKGHDGLYQGLSTATKDTYDALHMQALPPR | |
| MK signal sequence | METDTLLLWVLLLWVPGSTG | 2 |
| FLAG | DYKDDDDK | 3 |
| Full IL13 mutein | LTCLGGFASPGPVPPSTALRYLIEELVNITQNQKAPLCN | 20 |
| | GSMVWSINLTAGMYCAALESLINVSGCSAIEKTQRMLS | |
| | GFCPHKVSAGQFSSLHVRDTKIEVAQFVKDLLLHLKKL | |
| | FREGREN | |
| (G4S)x4 | GGGGSGGGGSGGGGSGGGGS | 10 |
| TGF-β scFv | QVQLVQSGAEVKKPGSSVKVSCKASGYTFSSNVISWVR | 11 |
| | QAPGQGLEWMGGVIPIVDIANYAQRFKGRVTITADEST | |
| | STTYMELSSLRSEDTAVYYCALPRAFVLDAMDYWGQG | |
| | TLVTVSSGGGGSGGGGSGGGGSETVLTQSPGTLSLSPG | |
| | ERATLSCRASQSLGSSYLAWYQQKPGQAPRLLIYGASS | |
| | RAPGIPDRFSGSGSGTDFTLTISRLEPEDFAVYYCQQYA | |
| | DSPITFGQGTRLEIK | |
| IgG4 hinge | ESKYGPPCPPCP | 12 |
| CD28tm | MFWVLVVVGGVLACYSLLVTVAFIIFWV | 6 |
| 4-1BB | KRGRKKLLYIFKQPFMRPVQTTQEEDGCSCRFPEEEEG | 7 |
| | GCEL | |
| CD3zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| Full-IL-13Rα2.28z | METDTLLLWVLLLWVPGSTGAGGSDYKDDDDKGGSL | 22 |
| CAR; (Murine kappa | TCLGGFASPGPVPPSTALRYLIEELVNITQNQKAPLCNG | |
| signal | SMVWSINLTAGMYCAALESLINVSGCSAIEKTQRMLSG | |
| sequence_FLAG_Full | FCPHKVSAGQFSSLHVRDTKIEVAQFVKDLLLHLKKLF | |
| IL13 mutein_IgG4 | REGRFNESKYGPPCPPCPAPEFEGGPSVFLFPPKPKDTL | |
| (L235E, | MISRTPEVTCVVVDVSQEDPEVQFNWYVDGVEVHNAK | |
| N297Q)_CD28tm_CD2 | TKPREEQFQSTYRVVSVLTVLHQDWLNGKEYKCKVSN | |
| 8cyto_CD3zeta) | KGLPSSIEKTISKAKGQPREPQVYTLPPSQEEMTKNQVS | |
| | LTCLVKGFYPSDIAVEWESNGQPENNYKTTPPVLDSDG | |
| | SFFLYSRLTVDKSRWQEGNVFSCSVMHEALHNHYTQK | |
| | SLSLSLGKMFWVLVVVGGVLACYSLLVTVAFIIFWVRS | |
| | KRSRGGHSDYMNMTPRRPGPTRKHYQPYAPPRDFAAY | |
| | RSGGGRVKFSRSADAPAYQQGQNQLYNELNLGRREEY | |
| | DVLDKRRGRDPEMGGKPRRKNPQEGLYNELQKDKMA | |
| | EAYSEIGMKGERRRGKGHDGLYQGLSTATKDTYDALH | |
| | MQALPPR | |
| Full-IL-13Rα2.28z | METDTLLLWVLLLWVPGSTGLTCLGGFASPGPVPPSTA | 144 |
| CAR; (Murine kappa | LRYLIEELVNITQNQKAPLCNGSMVWSINLTAGMYCAA | |
| signal sequence_ Full | LESLINVSGCSAIEKTQRMLSGFCPHKVSAGQFSSLHVR | |
| IL13 mutein_IgG4 | DTKIEVAQFVKDLLLHLKKLFREGRFNESKYGPPCPPCP | |
| (L235E, | APEFEGGPSVFLFPPKPKDTLMISRTPEVTCVVVDVSQE | |
| N297Q)_CD28tm_CD2 | DPEVQFNWYVDGVEVHNAKTKPREEQFQSTYRVVSVL | |
| 8cyto_CD3zeta) | TVLHQDWLNGKEYKCKVSNKGLPSSIEKTISKAKGQPR | |
| | EPQVYTLPPSQEEMTKNQVSLTCLVKGFYPSDIAVEWE | |
| | SNGQPENNYKTTPPVLDSDGSFFLYSRLTVDKSRWQEG | |
| | NVFSCSVMHEALHNHYTQKSLSLSLGKMFWVLVVVG | |
| | GVLACYSLLVTVAFIIFWVRSKRSRGGHSDYMNMTPRR | |
| | PGPTRKHYQPYAPPRDFAAYRSGGGRVKFSRSADAPA | |
| | YQQGQNQLYNELNLGRREEYDVLDKRRGRDPEMGGK | |
| | PRRKNPQEGLYNELQKDKMAEAYSEIGMKGERRRGKG | |
| | HDGLYQGLSTATKDTYDALHMQALPPR | |
| MK signal sequence | METDTLLLWVLLLWVPGSTG | 2 |
| FLAG | DYKDDDDK | 3 |
| Full IL13 mutein | LTCLGGFASPGPVPPSTALRYLIEELVNITQNQKAPLCN | 20 |
| | GSMVWSINLTAGMYCAALESLINVSGCSAIEKTQRMLS | |
| | GFCPHKVSAGQFSSLHVRDTKIEVAQFVKDLLLHLKKL | |
| | FREGREN | |
| IgG4 (L235E, N297Q) | ESKYGPPCPPCPAPEFEGGPSVFLFPPKPKDTLMISRTPE | 5 |
| | VTCVVVDVSQEDPEVQFNWYVDGVEVHNAKTKPREE | |
| | QFQSTYRVVSVLTVLHQDWLNGKEYKCKVSNKGLPSS | |
| | IEKTISKAKGQPREPQVYTLPPSQEEMTKNQVSLTCLVK | |
| | GFYPSDIAVEWESNGQPENNYKTTPPVLDSDGSFFLYSR | |
| | LTVDKSRWQEGNVFSCSVMHEALHNHYTQKSLSLSLG | |
| | K | |
| CD28tm | MFWVLVVVGGVLACYSLLVTVAFIIFWV | 6 |
| CD28cyto | RSKRSRGGHSDYMNMTPRRPGPTRKHYQPYAPPRDFA | 18 |
| | AYRS | |
| CD3zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| Full-IL-13Rα2/TGF- | METDTLLLWVLLLWVPGSTGAGGSDYKDDDDKGGSL | 23 |
| β.28z CAR; (Murine | TCLGGFASPGPVPPSTALRYLIEELVNITQNQKAPLCNG | |
| kappa signal | SMVWSINLTAGMYCAALESLINVSGCSAIEKTQRMLSG | |
| sequence_FLAG_Full | FCPHKVSAGQFSSLHVRDTKIEVAQFVKDLLLHLKKLF | |
| IL13 | REGRFNGGGGSGGGGSGGGGSGGGGSQVQLVQSGAE | |
| mutein_(G4S)x4_TGF- | VKKPGSSVKVSCKASGYTFSSNVISWVRQAPGQGLEW | |
| β scFv_IgG4 | MGGVIPIVDIANYAQRFKGRVTITADESTSTTYMELSSL | |
| hinge_CD28tm | RSEDTAVYYCALPRAFVLDAMDYWGQGTLVTVSSGG | |
| CD28cyto_CD3zeta) | GGSGGGGSGGGGSETVLTQSPGTLSLSPGERATLSCRA | |
| | SQSLGSSYLAWYQQKPGQAPRLLIYGASSRAPGIPDRFS | |
| | GSGSGTDFTLTISRLEPEDFAVYYCQQYADSPITFGQGT | |
| | RLEIKESKYGPPCPPCPMFWVLVVVGGVLACYSLLVTV | |
| | AFIIFWVRSKRSRGGHSDYMNMTPRRPGPTRKHYQPYA | |
| | PPRDFAAYRSGGGRVKFSRSADAPAYQQGQNQLYNEL | |
| | NLGRREEYDVLDKRRGRDPEMGGKPRRKNPQEGLYNE | |
| | LQKDKMAEAYSEIGMKGERRRGKGHDGLYQGLSTAT | |
| | KDTYDALHMQALPPR | |
| Full-IL-13Rα2/TGF- | METDTLLLWVLLLWVPGSTGLTCLGGFASPGPVPPSTA | 145 |
| β.28z CAR; (Murine | LRYLIEELVNITQNQKAPLCNGSMVWSINLTAGMYCAA | |
| kappa signal sequence_ | LESLINVSGCSAIEKTORMLSGFCPHKVSAGQFSSLHVR | |
| Full IL13 | DTKIEVAQFVKDLLLHLKKLFREGRFNGGGGSGGGGS | |
| mutein_(G4S)x4_TGF- | GGGGSGGGGSQVQLVQSGAEVKKPGSSVKVSCKASGY | |
| β scFv_IgG4 | TFSSNVISWVRQAPGQGLEWMGGVIPIVDIANYAQRFK | |
| hinge_CD28tm_ | GRVTITADESTSTTYMELSSLRSEDTAVYYCALPRAFVL | |
| CD28cyto_CD3zeta) | DAMDYWGQGTLVTVSSGGGGSGGGGSGGGGSETVLT | |
| | QSPGTLSLSPGERATLSCRASQSLGSSYLAWYQQKPGQ | |
| | APRLLIYGASSRAPGIPDRFSGSGSGTDFTLTISRLEPEDF | |
| | AVYYCQQYADSPITFGQGTRLEIKESKYGPPCPPCPMF | |
| | WVLVVVGGVLACYSLLVTVAFIIFWVRSKRSRGGHSD | |
| | YMNMTPRRPGPTRKHYQPYAPPRDFAAYRSGGGRVKF | |
| | SRSADAPAYQQGQNQLYNELNLGRREEYDVLDKRRGR | |
| | DPEMGGKPRRKNPQEGLYNELQKDKMAEAYSEIGMK | |
| | GERRRGKGHDGLYQGLSTATKDTYDALHMQALPPR | |
| MK signal sequence | METDTLLLWVLLLWVPGSTG | 2 |
| FLAG | DYKDDDDK | 3 |
| Full IL13 mutein | LTCLGGFASPGPVPPSTALRYLIEELVNITQNQKAPLCN | 20 |
| | GSMVWSINLTAGMYCAALESLINVSGCSAIEKTQRMLS | |
| | GFCPHKVSAGQFSSLHVRDTKIEVAQFVKDLLLHLKKL | |
| | FREGREN | |
| (G4S)x4 | GGGGSGGGGSGGGGSGGGGS | 10 |
| TGF-β scFv | QVQLVQSGAEVKKPGSSVKVSCKASGYTFSSNVISWVR | 11 |
| | QAPGQGLEWMGGVIPIVDIANYAQRFKGRVTITADEST | |
| | STTYMELSSLRSEDTAVYYCALPRAFVLDAMDYWGQG | |
| | TLVTVSSGGGGSGGGGSGGGGSETVLTQSPGTLSLSPG | |
| | ERATLSCRASQSLGSSYLAWYQQKPGQAPRLLIYGASS | |
| | RAPGIPDRFSGSGSGTDFTLTISRLEPEDFAVYYCQQYA | |
| | DSPITFGQGTRLEIK | |
| IgG4 hinge | ESKYGPPCPPCP | 12 |
| CD28tm | MFWVLVVVGGVLACYSLLVTVAFIIFWV | 6 |
| CD28cyto | RSKRSRGGHSDYMNMTPRRPGPTRKHYQPYAPPRDFA | 18 |
| | AYRS | |
| CD3zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| T2A | EGRGSLLTCGDVEENPGPR | 24 |
| (G4S)x3 | GGGGSGGGGSGGGGS | 28 |
| TGF-β scFv VH | QVQLVQSGAEVKKPGSSVKVSCKASGYTFSSNVISWVR | 29 |
| | QAPGQGLEWMGGVIPIVDIANYAQRFKGRVTITADEST | |
| | STTYMELSSLRSEDTAVYYCALPRAFVLDAMDYWGQG | |
| | TLVTVSS | |
| TGF-β scFv VL | ETVLTQSPGTLSLSPGERATLSCRASQSLGSSYLAWYQ | 30 |
| | QKPGQAPRLLIYGASSRAPGIPDRFSGSGSGTDFTLTISR | |
| | LEPEDFAVYYCQQYADSPITFGQGTRLEIK | |
| TGF-β scFv HCDR1 | SNVIS | 31 |
| TGF-β scFv HCDR2 | GVIPIVDIANYAQRFKG | 32 |
| TGF-β scFv HCDR3 | PRAFVLDAMDY | 33 |
| TGF-β scFv LCDR1 | RASQSLGSSYLA | 34 |
| TGF-β scFv LCDR2 | GASSRAP | 35 |
| TGF-β scFv LCDR3 | QQYADSPIT | 36 |
| IgG4 CH2 CH3 L235E | APEFEGGPSVFLFPPKPKDTLMISRTPEVTCVVVDVSQE | 37 |
| N297Q peptide spacer | DPEVQFNWYVDGVEVHNAKTKPREEQFQSTYRVVSVL | |
| | TVLHQDWLNGKEYKCKVSNKGLPSSIEKTISKAKGQPR | |
| | EPQVYTLPPSQEEMTKNQVSLTCLVKGFYPSDIAVEWE | |
| | SNGQPENNYKTTPPVLDSDGSFFLYSRLTVDKSRWQEG | |
| | NVFSCSVMHEALHNHYTQKSLSLSLGK | |
| EGFRVIII scFv | EIQLVQSGAEVKKPGESLRISCKGSGFNIEDYYIHWVRQ | 27 |
| | MPGKGLEWMGRIDPENDETKYGPIFQGHVTISADTSIN | |
| | TVYLQWSSLKASDTAMYYCAFRGGVYWGQGTTVTVS | |
| | SGSTSGSGKPGSGEGSTKGDVVMTQSPDSLAVSLGERA | |
| | TINCKSSQSLLDSDGKTYLNWLQQKPGQPPKRLISLVSK | |
| | LDSGVPDRFSGSGSGTDFTLTISSLQAEDVAVYYCWQG | |
| | THFPGTFGGGTKVEIK | |
| EGFRVIII scFv VH | EIQLVQSGAEVKKPGESLRISCKGSGFNIEDYYIHWVRQ | 38 |
| | MPGKGLEWMGRIDPENDETKYGPIFQGHVTISADTSIN | |
| | TVYLQWSSLKASDTAMYYCAFRGGVYWGQGTTVTVS | |
| | S | |
| EGFRVIII scFv VL | DVVMTQSPDSLAVSLGERATINCKSSQSLLDSDGKTYL | 39 |
| | NWLQQKPGQPPKRLISLVSKLDSGVPDRFSGSGSGTDF | |
| | TLTISSLQAEDVAVYYCWQGTHFPGTFGGGTKVEIK | |
| EGFRVIII scFv | DYYIH | 40 |
| HCDR1 | | |
| EGFRVIII scFv | RIDPENDETKYGPIFQG | 41 |
| HCDR2 | | |
| EGFRVIII scFv | RGGVY | 42 |
| HCDR3 | | |
| EGFRVIII scFv LCDR1 | KSSQSLLDSDGKTYLN | 43 |
| EGFRVIII scFv LCDR2 | LVSKLDS | 44 |
| EGFRVIII scFv LCDR3 | WQGTHFPGT | 45 |
| GD2 scFv | EVQLLQSGPELEKPGASVMISCKASGSSFTGYNMNWV | 26 |
| | RQNIGKSLEWIGAIDPYYGGTSYNQKFKGRATLTVDKS | |
| | SSTAYMHLKSLTSEDSAVYYCVSGMEYWGQGTSVTVS | |
| | SGSTSGSGKPGSGEGSTKGDVVMTQTPLSLPVSLGDQA | |
| | SISCRSSQSLVHRNGNTYLHWYLQKPGQSPKLLIHKVS | |
| | NRFSGVPDRFSGSGSGTDFTLKISRVEAEDLGVYFCSQS | |
| | THVPPLTFGAGTKLELKRA | |
| GD2 (14g2a scFv) VH | EVQLLQSGPELEKPGASVMISCKASGSSFTGYNMNWV | 46 |
| | RQNIGKSLEWIGAIDPYYGGTSYNQKFKGRATLTVDKS | |
| | SSTAYMHLKSLTSEDSAVYYCVSGMEYWGQGTSVTVS | |
| | S | |
| GD2 (14g2a scFv) VL | DVVMTQTPLSLPVSLGDQASISCRSSQSLVHRNGNTYL | 47 |
| | HWYLQKPGQSPKLLIHKVSNRFSGVPDRFSGSGSGTDF | |
| | TLKISRVEAEDLGVYFCSQSTHVPPLTFGAGTKLELKRA | |
| GD2scFv HCDR1 | GYNMN | 48 |
| GD2scFv HCDR2 | AIDPYYGGTSYNQKFKG | 49 |
| GD2scFv HCDR3 | GMEY | 50 |
| GD2scFv LCDR1 | RSSQSLVHRNGNTYLH | 51 |
| GD2scFv LCDR2 | KVSNRFS | 52 |
| GD2scFv LCDR3 | SQSTHVPPLT | 53 |
| TGF-β scFv VH #2 | EVOLVESGGGLVQPGGSLRLSCAASGYAFTNYLIEWVR | 54 |
| | QAPGKGLEWVGVINPGSGGSNYNEKFKGRATISADNS | |
| | KNTLYLQMNSLRAEDTAVYYCARSGGFYFDYWGQGT | |
| | LVTVSSASTKGPS | |
| TGF-β scFv VL #2 | DIQMTQSPSSLSASVGDRVTITCRASQSVLYSSNQKNYL | 55 |
| | AWYQQKPGKAPKLLIYWASTRESGVPSRFSGSGSGTDF | |
| | TLTISSLQPEDFATYYCHQYLSSDTFGQGTKVEIKRTVA | |
| TGF-β scFv #2 | GYAFTNYLIE | 56 |
| HCDR1 | | |
| TGF-β scFv #2 | VINPGSGGSNYNEKFKG | 57 |
| HCDR2 | | |
| TGF-β scFv #2 | SGGFYFDY | 58 |
| HCDR3 | | |
| TGF-β scFv #2 LCDR1 | RASQSVLYSSNQKNYLA | 59 |
| TGF-β scFv #2 LCDR2 | WASTRES | 60 |
| TGF-β scFv #2 LCDR3 | HQYLSSDT | 61 |
| TGF-β scFv VH #3 | EVOLVESGGGVVQPGRSLRLSCAASGFTFSSYGMHWV | 62 |
| | RQAPGKELEWVAVISYDGSIKYYADSVKGRFTISRDNS | |
| | KNTLYLQMNSLRAEDTAVYYCARTGEYSGYDTDPQYS | |
| | WGQGTTVTVSS | |
| TGF-β scFv VL #3 | EIVLTQSPSSLSASVGDRVTITCRSSQGIGDDLGWYQQK | 63 |
| | PGKAPILLIYGTSTLQSGVPSRFSGSGSGTDFTLTINSLQP | |
| | EDFATYYCLQDSNYPLTFGGGTRLEIK | |
| TGF-β scFv #3 | SYGMH | 64 |
| HCDR1 | | |
| TGF-β scFv #3 | VISYDGSIKYYADSVKG | 65 |
| HCDR2 | | |
| TGF-β scFv #3 | TGEYSGYDTDPQYS | 66 |
| HCDR3 | | |
| TGF-β scFv #3 LCDR1 | RSSQGIGDDLG | 67 |
| TGF-β scFv #3 LCDR2 | GTSTLQS | 68 |
| TGF-β scFv #3 LCDR3 | LQDSNYPLT | 69 |

### X. Examples

The following examples are included to demonstrate preferred aspects and embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. The Examples should not be construed as limiting in any way. The contents of all cited references (including literature references, issued patents, published patent applications, and GenBank Accession numbers as cited throughout this application) are hereby expressly incorporated by reference. When definitions of terms in documents that are incorporated by reference herein conflict with those used herein, the definitions used herein govern.

### Example 1: Treatment of Glioblastoma Multiforme with Multispecific Chimeric Antigen Receptors

Glioblastoma multiforme (GBM) is the most common type of primary brain tumors in adults, and the median survival period has remained at 12-16 months from the time of diagnosis over the past few decades. Conventional therapies such as surgery, chemotherapy, and radiation almost invariably fail to eradicate tumor, resulting in relapse within weeks or months. Consequently, GBM has been an active area of research for new treatment options such as adoptive T-cell therapy. Two major challenges have limited the efficacy of T-cell therapy for GBM thus far. First, the GBM tumor microenvironment is strongly immunosuppressive, characterized by a high level of transforming growth factor beta (TGF-β), which simultaneously promote tumor growth and potently suppress the function of T cells. Second, GBM tumors are highly heterogeneous in antigen expression, thus T cells engineered to target a single antigen are generally unable to recognize and eradicate all tumor cells present.

The inventors propose to overcome the two main challenges of adoptive T-cell therapy against GBM through the use of bispecific chimeric antigen receptor (CAR)-T cells that can simultaneously target a GBM-associated surface antigen and convert TGF-β from an immunosuppressive cytokine into a potent stimulant for the engineered T cells. Importantly, the TGF-β CAR can both inhibit endogenous TGF-β signaling (by competing against endogenous TGF-β receptors for binding to TGF-β ligands) and trigger T-cell activation in the presence of both soluble and immobilized TGF-β. The concept is that the TGF-β conversion function of the CAR-T cells could modify the tumor microenvironment, thus promoting the anti-tumor function of both the engineered T cells and endogenous immune cells.

The inventors have built a series of bispecific CARs that simultaneously respond to TGF-β plus IL-13Rα2, and antigen found on the surface of brain-tumor cells. Specifically, they have constructed the following bispecific CARs:

| | | SEQ |
|---|---|---|
| CAR | Sequence | ID NO: |
| SP-IL- | METDTLLLWVLLLWVPGSTGAGGSDYKDDDDKGGSSPGPV | 1 |
| 13Rα2.BBz | PPSTALRYLIEELVNITQNQKAPLCNGSMVWSINLTAGMYCA | |
| CAR; (Murine | ALESLINVSGCSAIEKTQRMLSGFCPHKVSAGQFSSLHVRDT | |
| kappa signal | KIEVAQFVKDLLLHLKKLFREGRFNESKYGPPCPPCPAPEFEG | |
| sequence_FLAG | GPSVFLFPPKPKDTLMISRTPEVTCVVVDVSQEDPEVQFNWY | |
| SP IL13 | VDGVEVHNAKTKPREEQFQSTYRVVSVLTVLHQDWLNGKE | |
| mutein_IgG4 | YKCKVSNKGLPSSIEKTISKAKGQPREPQVYTLPPSQEEMTK | |
| (L235E, | NQVSLTCLVKGFYPSDIAVEWESNGQPENNYKTTPPVLDSDG | |
| N297Q)_CD28t | SFFLYSRLTVDKSRWQEGNVFSCSVMHEALHNHYTQKSLSL | |
| m_4- | SLGKMFWVLVVVGGVLACYSLLVTVAFIIFWVKRGRKKLLY | |
| 1BB_CD3zeta) | IFKQPFMRPVQTTQEEDGCSCRFPEEEEGGCELRVKFSRSAD | |
| | APAYQQGQNQLYNELNLGRREEYDVLDKRRGRDPEMGGKP | |
| | RRKNPQEGLYNELQKDKMAEAYSEIGMKGERRRGKGHDGL | |
| | YQGLSTATKDTYDALHMQALPPR | |
| SP-IL- | METDTLLLWVLLLWVPGSTGAGGSDYKDDDDKGGSSPGPV | 9 |
| 13Rα2/TGF- | PPSTALRYLIEELVNITQNQKAPLCNGSMVWSINLTAGMYCA | |
| β.BBz CAR; | ALESLINVSGCSAIEKTQRMLSGFCPHKVSAGQFSSLHVRDT | |
| (Murine kappa | KIEVAQFVKDLLLHLKKLFREGRFNGGGGSGGGGSGGGGSG | |
| signal | GGGSQVQLVQSGAEVKKPGSSVKVSCKASGYTFSSNVISWV | |
| sequence_FLAG | RQAPGQGLEWMGGVIPIVDIANYAQRFKGRVTITADESTSTT | |
| SP IL13 | YMELSSLRSEDTAVYYCALPRAFVLDAMDYWGQGTLVTVS | |
| mutein_(G4S)x4 | SGGGGSGGGGSGGGGSETVLTQSPGTLSLSPGERATLSCRAS | |
| TGF-β | QSLGSSYLAWYQQKPGQAPRLLIYGASSRAPGIPDRFSGSGS | |
| scFv_IgG4 | GTDFTLTISRLEPEDFAVYYCQQYADSPITFGQGTRLEIKESK | |
| hinge_CD28tm_4 | YGPPCPPCPMFWVLVVVGGVLACYSLLVTVAFIIFWVKRGR | |
| -1BB_CD3zeta) | KKLLYIFKQPFMRPVQTTQEEDGCSCRFPEEEEGGCELRVKF | |
| | SRSADAPAYQQGQNQLYNELNLGRREEYDVLDKRRGRDPE | |
| | MGGKPRRKNPQEGLYNELQKDKMAEAYSEIGMKGERRRGK | |
| | GHDGLYQGLSTATKDTYDALHMQALPPR | |
| SP-IL- | METDTLLLWVLLLWVPGSTGAGGSDYKDDDDKGGSSPGPV | 13 |
| 13Rα2.BBz (KR) | PPSTALRYLIEELVNITQNQKAPLCNGSMVWSINLTAGMYCA | |
| CAR; (Murine | ALESLINVSGCSAIEKTQRMLSGFCPHKVSAGQFSSLHVRDT | |
| kappa signal | KIEVAQFVKDLLLHLKKLFREGRFNESKYGPPCPPCPAPEFEG | |
| sequence_FLAG | GPSVFLFPPKPKDTLMISRTPEVTCVVVDVSQEDPEVQFNWY | |
| _SP IL13 | VDGVEVHNAKTKPREEQFQSTYRVVSVLTVLHQDWLNGKE | |
| mutein_IgG4 | YKCKVSNKGLPSSIEKTISKAKGQPREPQVYTLPPSQEEMTK | |
| (L235E, | NQVSLTCLVKGFYPSDIAVEWESNGQPENNYKTTPPVLDSDG | |
| N297Q)_CD28t | SFFLYSRLTVDKSRWQEGNVFSCSVMHEALHNHYTQKSLSL | |
| m_4- | SLGKMFWVLVVVGGVLACYSLLVTVAFIIFWVRRGRRRLLY | |
| 1BB(KR)_CD3ze | IFRQPFMRPVQTTQEEDGCSCRFPEEEEGGCELRVRFSRSADA | |
| ta(KR)) | PAYQQGQNQLYNELNLGRREEYDVLDRRRGRDPEMGGRPR | |
| | RRNPQEGLYNELQRDRMAEAYSEIGMRGERRRGRGHDGLY | |
| | QGLSTATRDTYDALHMQALPPR | |
| SP-IL- | METDTLLLWVLLLWVPGSTGAGGSDYKDDDDKGGSSPGPV | 16 |
| 13Rα2/TGF- | PPSTALRYLIEELVNITQNQKAPLCNGSMVWSINLTAGMYCA | |
| β.BBz (KR) | ALESLINVSGCSAIEKTQRMLSGFCPHKVSAGQFSSLHVRDT | |
| CAR; (Murine | KIEVAQFVKDLLLHLKKLFREGRFNGGGGSGGGGSGGGGSG | |
| kappa signal | GGGSQVQLVQSGAEVKKPGSSVKVSCKASGYTFSSNVISWV | |
| sequence_FLAG | RQAPGQGLEWMGGVIPIVDIANYAQRFKGRVTITADESTSTT | |
| SP IL13 | YMELSSLRSEDTAVYYCALPRAFVLDAMDYWGQGTLVTVS | |
| mutein_(G4S)x4 | SGGGGSGGGGSGGGGSETVLTQSPGTLSLSPGERATLSCRAS | |
| TGF-β | QSLGSSYLAWYQQKPGQAPRLLIYGASSRAPGIPDRFSGSGS | |
| scFv_IgG4 | GTDFTLTISRLEPEDFAVYYCQQYADSPITFGQGTRLEIKESK | |
| hinge_ | YGPPCPPCPMFWVLVVVGGVLACYSLLVTVAFIIFWVRRGR | |
| CD28tm_4- | RRLLYIFRQPFMRPVQTTQEEDGCSCRFPEEEEGGCELRVRFS | |
| 1BB(KR)_CD3ze | RSADAPAYQQGQNQLYNELNLGRREEYDVLDRRRGRDPEM | |
| ta(KR)) | GGRPRRRNPQEGLYNELQRDRMAEAYSEIGMRGERRRGRG | |
| | HDGLYQGLSTATRDTYDALHMQALPPR | |
| SP-IL-13Rα2.28z | METDTLLLWVLLLWVPGSTGAGGSDYKDDDDKGGSSPGPV | 17 |
| CAR; (Murine | PPSTALRYLIEELVNITQNQKAPLCNGSMVWSINLTAGMYCA | |
| kappa signal | ALESLINVSGCSAIEKTQRMLSGFCPHKVSAGQFSSLHVRDT | |
| sequence_FLAG | KIEVAQFVKDLLLHLKKLFREGRFNESKYGPPCPPCPAPEFEG | |
| SP IL13 | GPSVFLFPPKPKDTLMISRTPEVTCVVVDVSQEDPEVQFNWY | |
| mutein_IgG4 | VDGVEVHNAKTKPREEQFQSTYRVVSVLTVLHQDWLNGKE | |
| (L235E, | YKCKVSNKGLPSSIEKTISKAKGQPREPQVYTLPPSQEEMTK | |
| N297Q)_CD28t | NQVSLTCLVKGFYPSDIAVEWESNGQPENNYKTTPPVLDSDG | |
| m_CD28cyto_C | SFFLYSRLTVDKSRWQEGNVFSCSVMHEALHNHYTQKSLSL | |
| D3zeta) | SLGKMFWVLVVVGGVLACYSLLVTVAFIIFWVRSKRSRGGH | |
| | SDYMNMTPRRPGPTRKHYQPYAPPRDFAAYRSGGGRVKFSR | |
| | SADAPAYQQGQNQLYNELNLGRREEYDVLDKRRGRDPEMG | |
| | GKPRRKNPQEGLYNELQKDKMAEAYSEIGMKGERRRGKGH | |
| | DGLYQGLSTATKDTYDALHMQALPPR | |
| SP-IL- | METDTLLLWVLLLWVPGSTGAGGSDYKDDDDKGGSSPGPV | 25 |
| 13Rα2/TGF- | PPSTALRYLIEELVNITQNQKAPLCNGSMVWSINLTAGMYCA | |
| β.28z CAR; | ALESLINVSGCSAIEKTQRMLSGFCPHKVSAGQFSSLHVRDT | |
| (Murine kappa | KIEVAQFVKDLLLHLKKLFREGRFNGGGGSGGGGSGGGGSG | |
| signal | GGGSQVQLVQSGAEVKKPGSSVKVSCKASGYTFSSNVISWV | |
| sequence_FLAG | RQAPGQGLEWMGGVIPIVDIANYAQRFKGRVTITADESTSTT | |
| SP IL13 | YMELSSLRSEDTAVYYCALPRAFVLDAMDYWGQGTLVTVS | |
| mutein_(G4S)x4 | SGGGGSGGGGSGGGGSETVLTQSPGTLSLSPGERATLSCRAS | |
| TGF-β | QSLGSSYLAWYQQKPGQAPRLLIYGASSRAPGIPDRFSGSGS | |
| scFv_IgG4 | GTDFTLTISRLEPEDFAVYYCQQYADSPITFGQGTRLEIKESK | |
| hinge_CD28tm_ | YGPPCPPCPMFWVLVVVGGVLACYSLLVTVAFIIFWVRSKRS | |
| CD28cyto_CD3z | RGGHSDYMNMTPRRPGPTRKHYQPYAPPRDFAAYRSGGGR | |
| eta) | VKFSRSADAPAYQQGQNQLYNELNLGRREEYDVLDKRRGR | |
| | DPEMGGKPRRKNPQEGLYNELQKDKMAEAYSEIGMKGERR | |
| | RGKGHDGLYQGLSTATKDTYDALHMQALPPR | |
| Murine kappa | METDTLLLWVLLLWVPGSTGAGGSDYKDDDDKGGSSPGPV | 131 |
| signal | PPSTALRYLIEELVNITQNQKAPLCNGSMVWSINLTAGMYCA | |
| sequence_FLAG | ALESLINVSGCSAIEKTQRMLSGFCPHKVSAGQFSSLHVRDT | |
| SP IL13 | KIEVAQFVKDLLLHLKKLFREGRFNESKYGPPCPPCPAPEFEG | |
| mutein_IgG4 | GPSVFLFPPKPKDTLMISRTPEVTCVVVDVSQEDPEVQFNWY | |
| (L235E, N297Q)_ | VDGVEVHNAKTKPREEQFQSTYRVVSVLTVLHQDWLNGKE | |
| CD28tm_4- | YKCKVSNKGLPSSIEKTISKAKGQPREPQVYTLPPSQEEMTK | |
| 1BB_CD3zeta_T | NQVSLTCLVKGFYPSDIAVEWESNGQPENNYKTTPPVLDSDG | |
| 2A_HA_TGFBR | SFFLYSRLTVDKSRWQEGNVFSCSVMHEALHNHYTQKSLSL | |
| 2tr(DNR) | SLGKMFWVLVVVGGVLACYSLLVTVAFIIFWVKRGRKKLLY | |
| | IFKQPFMRPVQTTQEEDGCSCRFPEEEEGGCELRVKFSRSAD | |
| | APAYQQGQNQLYNELNLGRREEYDVLDKRRGRDPEMGGKP | |
| | RRKNPQEGLYNELQKDKMAEAYSEIGMKGERRRGKGHDGL | |
| | YQGLSTATKDTYDALHMQALPPRLEGGGEGRGSLLTCGDVE | |
| | ENPGPRMGRGLLRGLWPLHIVLWTRIASTIPPYPYDVPDYAH | |
| | VQKSVNNDMIVTDNNGAVKFPQLCKFCDVRFSTCDNQKSC | |
| | MSNCSITSICEKPQEVCVAVWRKNDENITLETVCHDPKLPYH | |
| | DFILEDAASPKCIMKEKKKPGETFFMCSCSSDECNDNIIFSEEY | |
| | NTSNPDLLLVIFQVTGISLLPPLGVAISVIIIFYCYRVNRQQKLS | |
| | S | |
| Full IL- | METDTLLLWVLLLWVPGSTGAGGSDYKDDDDKGGSLTCLG | 19 |
| 13Rα2.BBz | GFASPGPVPPSTALRYLIEELVNITQNQKAPLCNGSMVWSINL | |
| CAR; (Murine | TAGMYCAALESLINVSGCSAIEKTQRMLSGFCPHKVSAGQFS | |
| kappa signal | SLHVRDTKIEVAQFVKDLLLHLKKLFREGRFNESKYGPPCPP | |
| sequence_FLAG | CPAPEFEGGPSVFLFPPKPKDTLMISRTPEVTCVVVDVSQEDP | |
| Full IL13 | EVQFNWYVDGVEVHNAKTKPREEQFQSTYRVVSVLTVLHQ | |
| mutein_IgG4 | DWLNGKEYKCKVSNKGLPSSIEKTISKAKGQPREPQVYTLPP | |
| (L235E, | SQEEMTKNQVSLTCLVKGFYPSDIAVEWESNGQPENNYKTT | |
| N297Q)_CD28tm_ | PPVLDSDGSFFLYSRLTVDKSRWQEGNVFSCSVMHEALHNH | |
| 4-1BB_ | YTQKSLSLSLGKMFWVLVVVGGVLACYSLLVTVAFIIFWVK | |
| CD3zeta) | RGRKKLLYIFKQPFMRPVQTTQEEDGCSCRFPEEEEGGCELR | |
| | VKFSRSADAPAYQQGQNQLYNELNLGRREEYDVLDKRRGR | |
| | DPEMGGKPRRKNPQEGLYNELQKDKMAEAYSEIGMKGERR | |
| | RGKGHDGLYQGLSTATKDTYDALHMQALPPR | |
| Full-IL- | METDTLLLWVLLLWVPGSTGAGGSDYKDDDDKGGSLTCLG | 21 |
| 13Rα2/TGF- | GFASPGPVPPSTALRYLIEELVNITQNQKAPLCNGSMVWSINL | |
| β.BBz CAR; | TAGMYCAALESLINVSGCSAIEKTQRMLSGFCPHKVSAGQFS | |
| (Murine kappa | SLHVRDTKIEVAQFVKDLLLHLKKLFREGRFNGGGGSGGGG | |
| signal | SGGGGSGGGGSQVQLVQSGAEVKKPGSSVKVSCKASGYTFS | |
| sequence_FLAG | SNVISWVRQAPGQGLEWMGGVIPIVDIANYAQRFKGRVTITA | |
| Full IL13 | DESTSTTYMELSSLRSEDTAVYYCALPRAFVLDAMDYWGQG | |
| mutein_(G4S)x4 | TLVTVSSGGGGSGGGGSGGGGSETVLTQSPGTLSLSPGERAT | |
| TGF-β | LSCRASQSLGSSYLAWYQQKPGQAPRLLIYGASSRAPGIPDR | |
| scFv_IgG4 | FSGSGSGTDFTLTISRLEPEDFAVYYCQQYADSPITFGQGTRL | |
| hinge_CD28tm_4 | EIKESKYGPPCPPCPMFWVLVVVGGVLACYSLLVTVAFIIFW | |
| -1BB_CD3zeta) | VKRGRKKLLYIFKQPFMRPVQTTQEEDGCSCRFPEEEEGGCE | |
| | LRVKFSRSADAPAYQQGQNQLYNELNLGRREEYDVLDKRR | |
| | GRDPEMGGKPRRKNPQEGLYNELQKDKMAEAYSEIGMKGE | |
| | RRRGKGHDGLYQGLSTATKDTYDALHMQALPPR | |
| Murine kappa | METDTLLLWVLLLWVPGSTGAGGSDYKDDDDKGGSLTCLG | 132 |
| signal | GFASPGPVPPSTALRYLIEELVNITQNQKAPLCNGSMVWSINL | |
| sequence_FLAG | TAGMYCAALESLINVSGCSAIEKTQRMLSGFCPHKVSAGQFS | |
| Full IL13 | SLHVRDTKIEVAQFVKDLLLHLKKLFREGRFNESKYGPPCPP | |
| mutein_IgG4 | CPAPEFEGGPSVFLFPPKPKDTLMISRTPEVTCVVVDVSQEDP | |
| (L235E, | EVQFNWYVDGVEVHNAKTKPREEQFQSTYRVVSVLTVLHQ | |
| N297Q)_CD28t | DWLNGKEYKCKVSNKGLPSSIEKTISKAKGQPREPQVYTLPP | |
| m_4-1BB | SQEEMTKNQVSLTCLVKGFYPSDIAVEWESNGQPENNYKTT | |
| (KR)_CD3zeta | PPVLDSDGSFFLYSRLTVDKSRWQEGNVFSCSVMHEALHNH | |
| (KR) | YTQKSLSLSLGKMFWVLVVVGGVLACYSLLVTVAFIIFWVR | |
| | RGRRRLLYIFRQPFMRPVQTTQEEDGCSCRFPEEEEGGCELR | |
| | VRFSRSADAPAYQQGQNQLYNELNLGRREEYDVLDRRRGR | |
| | DPEMGGRPRRRNPQEGLYNELQRDRMAEAYSEIGMRGERRR | |
| | GRGHDGLYQGLSTATRDTYDALHMQALPPR | |
| Murine kappa | METDTLLLWVLLLWVPGSTGAGGSDYKDDDDKGGSLTCLG | 133 |
| signal | GFASPGPVPPSTALRYLIEELVNITQNQKAPLCNGSMVWSINL | |
| sequence_FLAG | TAGMYCAALESLINVSGCSAIEKTQRMLSGFCPHKVSAGQFS | |
| Full IL13 | SLHVRDTKIEVAQFVKDLLLHLKKLFREGRFNGGGGSGGGG | |
| mutein_(G4S)x4 | SGGGGSGGGGSQVQLVQSGAEVKKPGSSVKVSCKASGYTFS | |
| TGF-β | SNVISWVRQAPGQGLEWMGGVIPIVDIANYAQRFKGRVTITA | |
| scFv_IgG4 | DESTSTTYMELSSLRSEDTAVYYCALPRAFVLDAMDYWGQG | |
| hinge_CD28tm_ | TLVTVSSGGGGSGGGGSGGGGSETVLTQSPGTLSLSPGERAT | |
| 4-1BB | LSCRASQSLGSSYLAWYQQKPGQAPRLLIYGASSRAPGIPDR | |
| (KR)_CD3zeta | FSGSGSGTDFTLTISRLEPEDFAVYYCQQYADSPITFGQGTRL | |
| (KR) | EIKESKYGPPCPPCPMFWVLVVVGGVLACYSLLVTVAFIIFW | |
| | VRRGRRRLLYIFRQPFMRPVQTTQEEDGCSCRFPEEEEGGCE | |
| | LRVRFSRSADAPAYQQGQNQLYNELNLGRREEYDVLDRRRG | |
| | RDPEMGGRPRRRNPQEGLYNELQRDRMAEAYSEIGMRGERR | |
| | RGRGHDGLYQGLSTATRDTYDALHMQALPPR | |
| Full-IL- | METDTLLLWVLLLWVPGSTGAGGSDYKDDDDKGGSLTCLG | 22 |
| 13Rα2.28z CAR; | GFASPGPVPPSTALRYLIEELVNITQNQKAPLCNGSMVWSINL | |
| (Murine kappa | TAGMYCAALESLINVSGCSAIEKTQRMLSGFCPHKVSAGQFS | |
| signal | SLHVRDTKIEVAQFVKDLLLHLKKLFREGRFNESKYGPPCPP | |
| sequence_FLAG | CPAPEFEGGPSVFLFPPKPKDTLMISRTPEVTCVVVDVSQEDP | |
| Full IL13 | EVQFNWYVDGVEVHNAKTKPREEQFQSTYRVVSVLTVLHQ | |
| mutein_IgG4 | DWLNGKEYKCKVSNKGLPSSIEKTISKAKGQPREPQVYTLPP | |
| (L235E, | SQEEMTKNQVSLTCLVKGFYPSDIAVEWESNGQPENNYKTT | |
| N297Q)_CD28t | PPVLDSDGSFFLYSRLTVDKSRWQEGNVFSCSVMHEALHNH | |
| m_CD28cyto_C | YTQKSLSLSLGKMFWVLVVVGGVLACYSLLVTVAFIIFWVR | |
| D3zeta) | SKRSRGGHSDYMNMTPRRPGPTRKHYQPYAPPRDFAAYRSG | |
| | GGRVKFSRSADAPAYQQGQNQLYNELNLGRREEYDVLDKR | |
| | RGRDPEMGGKPRRKNPQEGLYNELQKDKMAEAYSEIGMKG | |
| | ERRRGKGHDGLYQGLSTATKDTYDALHMQALPPR | |
| Full-IL- | METDTLLLWVLLLWVPGSTGAGGSDYKDDDDKGGSLTCLG | 23 |
| 13Rα2/TGF- | GFASPGPVPPSTALRYLIEELVNITQNQKAPLCNGSMVWSINL | |
| β.28z CAR; | TAGMYCAALESLINVSGCSAIEKTQRMLSGFCPHKVSAGQFS | |
| (Murine kappa | SLHVRDTKIEVAQFVKDLLLHLKKLFREGRFNGGGGSGGGG | |
| signal | SGGGGSGGGGSQVQLVQSGAEVKKPGSSVKVSCKASGYTFS | |
| sequence_FLAG | SNVISWVRQAPGQGLEWMGGVIPIVDIANYAQRFKGRVTITA | |
| Full IL13 | DESTSTTYMELSSLRSEDTAVYYCALPRAFVLDAMDYWGQG | |
| mutein_(G4S)x4 | TLVTVSSGGGGSGGGGSGGGGSETVLTQSPGTLSLSPGERAT | |
| TGF-β | LSCRASQSLGSSYLAWYQQKPGQAPRLLIYGASSRAPGIPDR | |
| scFv_IgG4 | FSGSGSGTDFTLTISRLEPEDFAVYYCQQYADSPITFGQGTRL | |
| hinge_CD28tm | EIKESKYGPPCPPCPMFWVLVVVGGVLACYSLLVTVAFIIFW | |
| CD28cyto_CD3z | VRSKRSRGGHSDYMNMTPRRPGPTRKHYQPYAPPRDFAAYR | |
| eta) | SGGGRVKFSRSADAPAYQQGQNQLYNELNLGRREEYDVLD | |
| | KRRGRDPEMGGKPRRKNPQEGLYNELQKDKMAEAYSEIGM | |
| | KGERRRGKGHDGLYQGLSTATKDTYDALHMQALPPR | |
| Murine kappa | METDTLLLWVLLLWVPGSTGAGGSDYKDDDDKGGSLTCLG | 134 |
| signal | GFASPGPVPPSTALRYLIEELVNITQNQKAPLCNGSMVWSINL | |
| sequence_FLAG | TAGMYCAALESLINVSGCSAIEKTQRMLSGFCPHKVSAGQFS | |
| Full IL13 | SLHVRDTKIEVAQFVKDLLLHLKKLFREGRFNESKYGPPCPP | |
| mutein_IgG4 | CPAPEFEGGPSVFLFPPKPKDTLMISRTPEVTCVVVDVSQEDP | |
| (L235E, N297Q). | EVQFNWYVDGVEVHNAKTKPREEQFQSTYRVVSVLTVLHQ | |
| CD28tm_4- | DWLNGKEYKCKVSNKGLPSSIEKTISKAKGQPREPQVYTLPP | |
| 1BB_CD3zeta_T2A_ | SQEEMTKNQVSLTCLVKGFYPSDIAVEWESNGQPENNYKTT | |
| HA_TGFBR | PPVLDSDGSFFLYSRLTVDKSRWQEGNVFSCSVMHEALHNH | |
| 2tr(DNR) | YTQKSLSLSLGKMFWVLVVVGGVLACYSLLVTVAFIIFWVK | |
| | RGRKKLLYIFKQPFMRPVQTTQEEDGCSCRFPEEEEGGCELR | |
| | VKFSRSADAPAYQQGQNQLYNELNLGRREEYDVLDKRRGR | |
| | DPEMGGKPRRKNPQEGLYNELQKDKMAEAYSEIGMKGERR | |
| | RGKGHDGLYQGLSTATKDTYDALHMQALPPRLEGGGEGRG | |
| | SLLTCGDVEENPGPRMGRGLLRGLWPLHIVLWTRIASTIPPYP | |
| | YDVPDYAHVQKSVNNDMIVTDNNGAVKFPQLCKFCDVRFS | |
| | TCDNQKSCMSNCSITSICEKPQEVCVAVWRKNDENITLETVC | |
| | HDPKLPYHDFILEDAASPKCIMKEKKKPGETFFMCSCSSDEC | |
| | NDNIIFSEEYNTSNPDLLLVIFQVTGISLLPPLGVAISVIIIFYCY | |
| | RVNRQQKLSS | |
| Murine kappa | METDTLLLWVLLLWVPGSTGAGGSDYKDDDDKGGSSPGPV | 135 |
| signal | PPSTALRYLIEELVNITQNQKAPLCNGSMVWSINLTAGMYCA | |
| sequence_FLAG | ALESLINVSGCSAIEKTQRMLSGFCPHKVSAGQFSSLHVRDT | |
| SP IL13 | KIEVAQFVKDLLLHLKKLFREGRFNESKYGPPCPPCPAPEFEG | |
| mutein_IgG4 | GPSVFLFPPKPKDTLMISRTPEVTCVVVDVSQEDPEVQFNWY | |
| (L235E, N297Q). | VDGVEVHNAKTKPREEQFQSTYRVVSVLTVLHQDWLNGKE | |
| CD28tm_CD28cyto_ | YKCKVSNKGLPSSIEKTISKAKGQPREPQVYTLPPSQEEMTK | |
| CD3zeta_T2A_ | NQVSLTCLVKGFYPSDIAVEWESNGQPENNYKTTPPVLDSDG | |
| HA_TGFBR2tr | SFFLYSRLTVDKSRWQEGNVFSCSVMHEALHNHYTQKSLSL | |
| (DNR) | SLGKMFWVLVVVGGVLACYSLLVTVAFIIFWVRSKRSRGGH | |
| | SDYMNMTPRRPGPTRKHYQPYAPPRDFAAYRSGGGRVKFSR | |
| | SADAPAYQQGQNQLYNELNLGRREEYDVLDKRRGRDPEMG | |
| | GKPRRKNPQEGLYNELQKDKMAEAYSEIGMKGERRRGKGH | |
| | DGLYQGLSTATKDTYDALHMQALPPRLEGGGEGRGSLLTCG | |
| | DVEENPGPRMGRGLLRGLWPLHIVLWTRIASTIPPYPYDVPD | |
| | YAHVQKSVNNDMIVTDNNGAVKFPQLCKFCDVRFSTCDNQ | |
| | KSCMSNCSITSICEKPQEVCVAVWRKNDENITLETVCHDPKL | |
| | PYHDFILEDAASPKCIMKEKKKPGETFFMCSCSSDECNDNIIF | |
| | SEEYNTSNPDLLLVIFQVTGISLLPPLGVAISVIIIFYCYRVNRQ | |
| | QKLSS | |

All of the constructs contain, from N terminal to C terminal of the protein, the following components: murine kappa chain signal sequence, binding domains each separated by (Gly4 Ser1)×4, IgG4 hinge, CD28 transmembrane domain, and CD3ζ cytoplasmic domain. Some CARs contain CD28 cytoplasmic domain between the transmembrane and CD3ζ domains; others contain 4-1BB cytoplasmic domain instead of CD28 cytoplasmic domain.

Constructs 7, 14, and 15 above co-express the single-input IL-13Rα2 CAR with a dominant-negative TGF-β receptor (DNR), which is TGF-β receptor chain 2 missing its cytoplasmic domain. These constructs are built as controls to compare against the IL-13Rα2/TGF-β bispecific CARs.

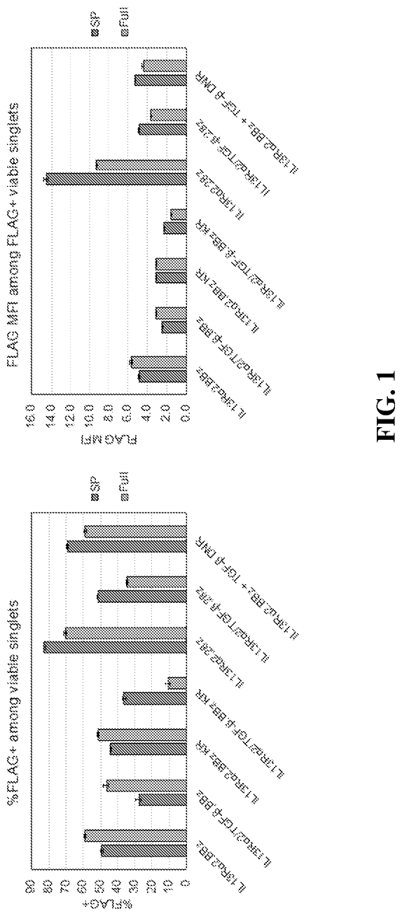

T cells were transduced with a panel of single-input IL-13Rα2 or bispecific IL-13Rα2/TGF-β CARs, bearing either an SP dipeptide or LTCLGGFASP (“Full”) polypeptide at the N-terminus of the IL-13 mutein. Each CAR was fused to an N-terminal FLAG tag to enable surface detection by antibody staining. On day 7 of culture, transduced T cells were stained for surface expression of FLAG-tagged CARs. The relative strength of CAR expression for IL-13 muteins with an N-terminal SP versus full N-terminus was construct-dependent. Averages of triplicates are shown, with error bars representing ±1 standard deviation. (FIG. 1).

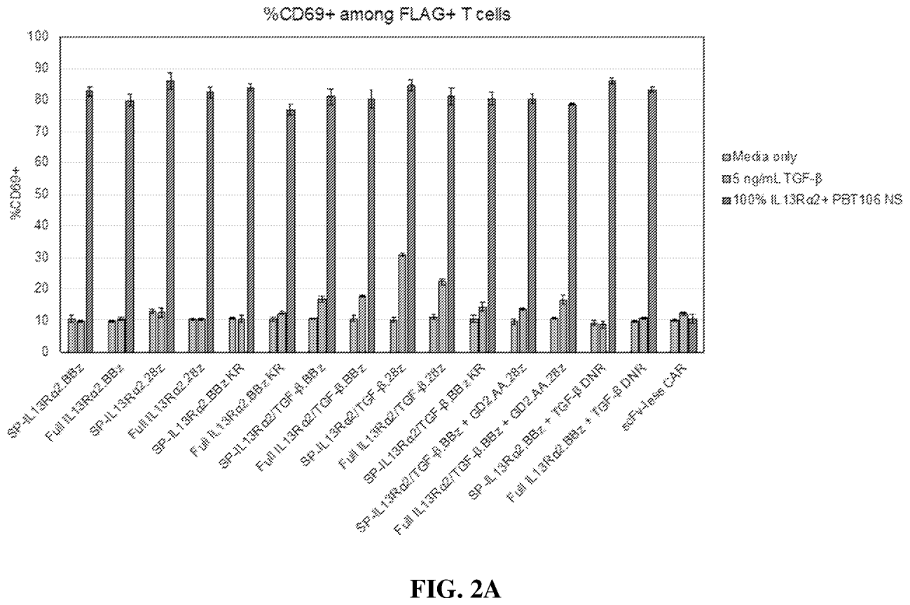

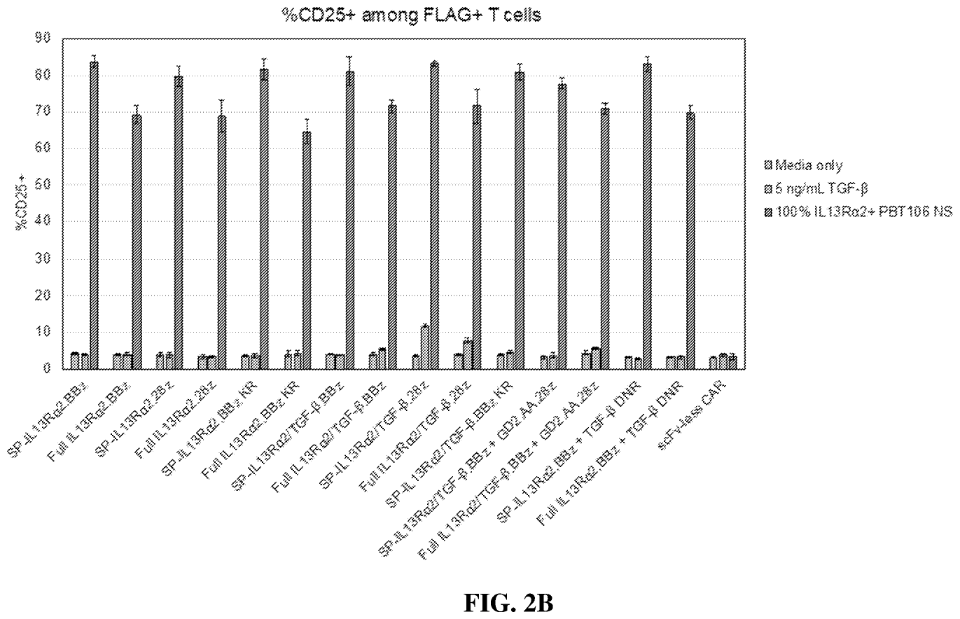

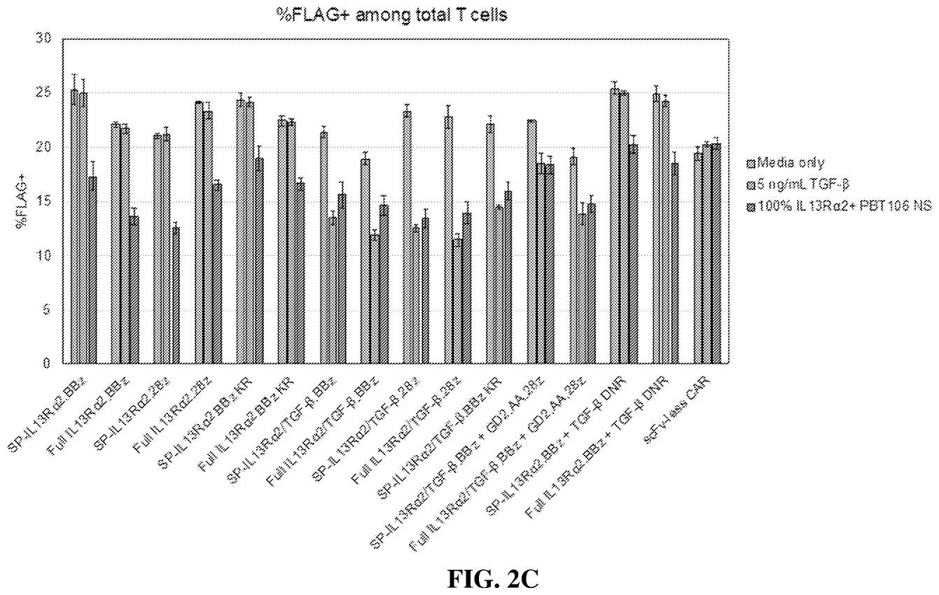

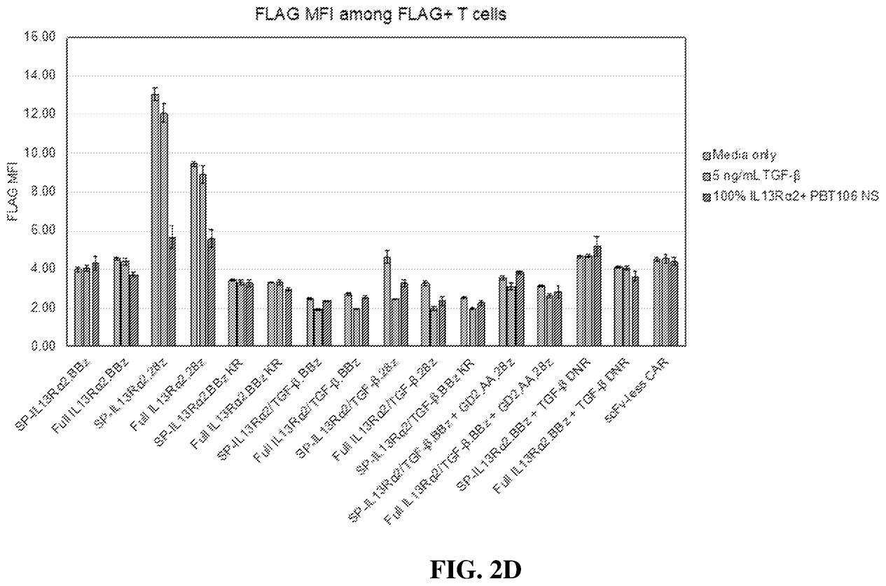

To evaluate whether CARs signal in response to antigen, CAR-T cells were cultured for 21 hours in either media alone, or in the presence of 5 ng/mL recombinant human TGF-β1 or IL-13Rα2+ PBT106 neurospheres, respectively. T cells were subsequently stained for surface expression of CD69 (FIG. 2A), CD25 (FIG. 2B), and FLAG (FIG. 2C,D). Both single-input IL-13Rα2 and bispecific IL-13Rα2/TGF-β CAR-T cells are strongly activated by antigen-expressing PBT106 tumor cells, while only bispecific IL-13Rα2/TGF-β CARs are activated by TGF-β, as evidenced by upregulation of CD69 and CD25, and downregulation of surface FLAG expression (FIG. 2A-D). Moreover, CARs with an N-terminal SP conferred greater CD25 upregulation compared to CARs with the full IL-13 mutein N-terminus, suggesting that the shorter SP N-terminus of the IL-13 mutein confers greater functionality compared to the full N-terminus. Averages of triplicates are shown, with error bars representing ±1 standard deviation.

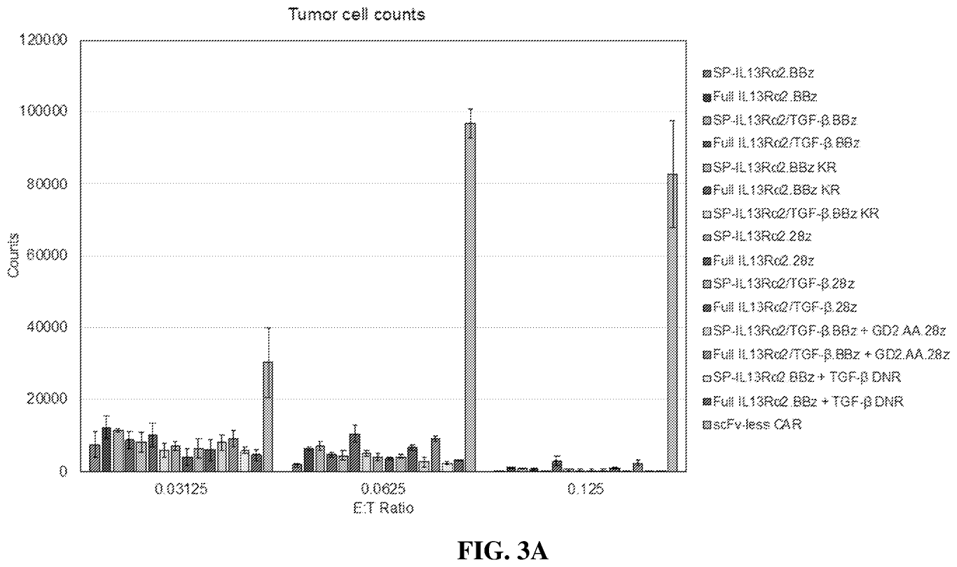

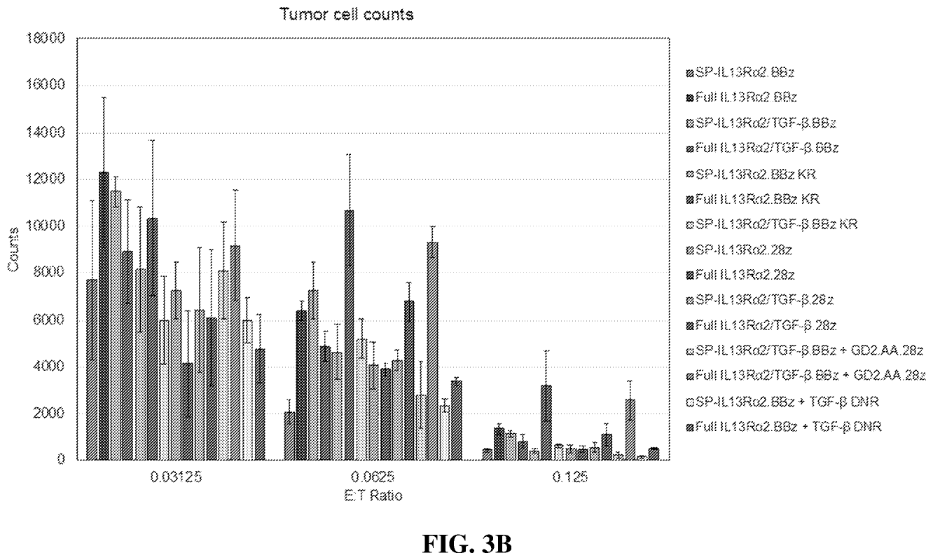

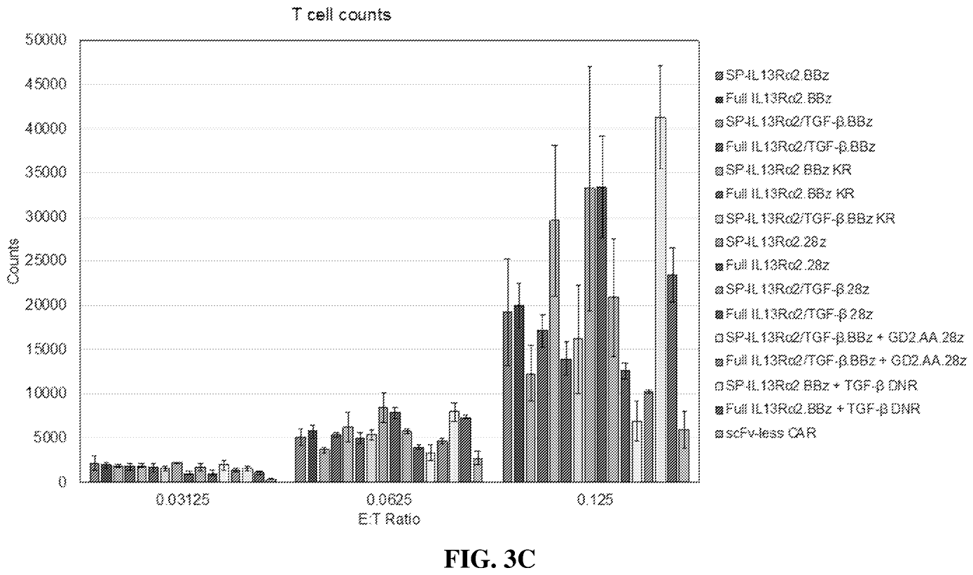

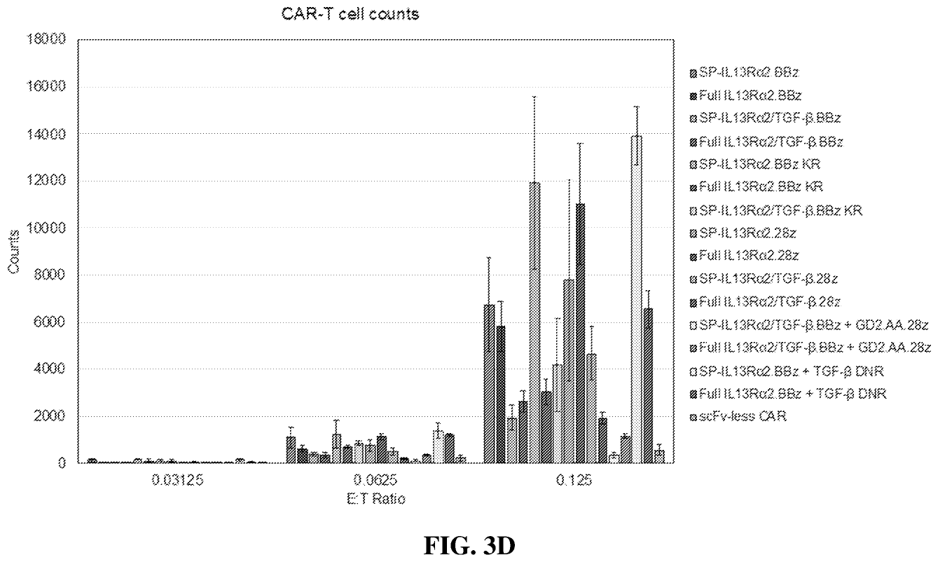

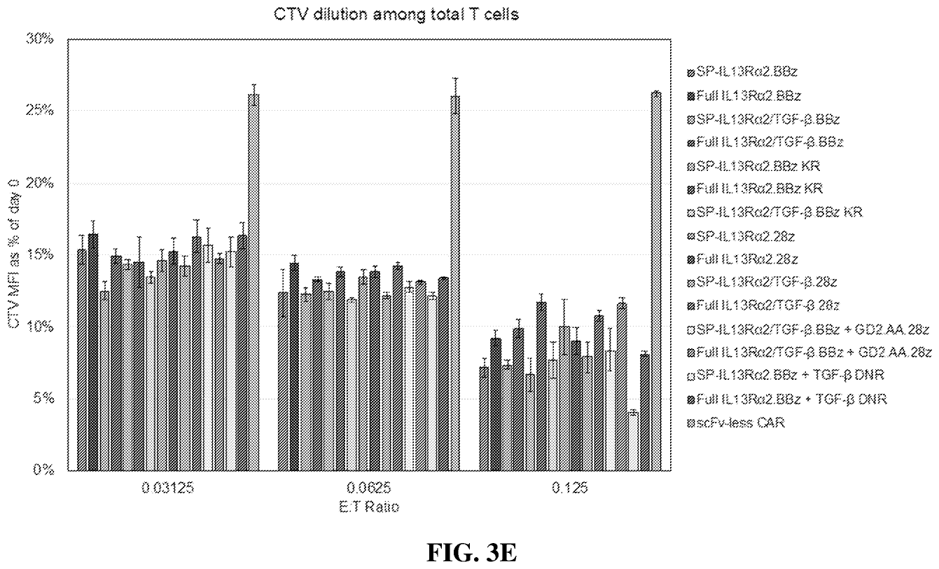

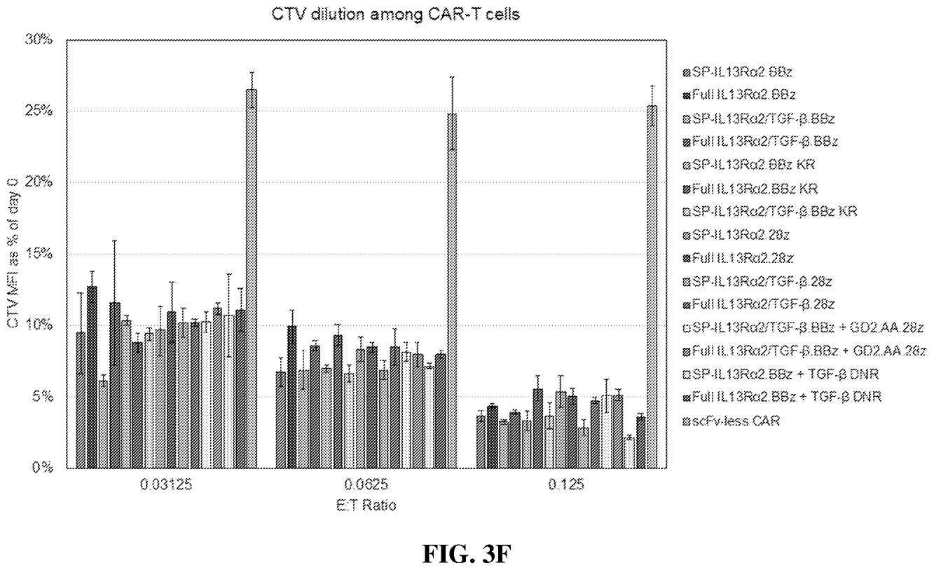

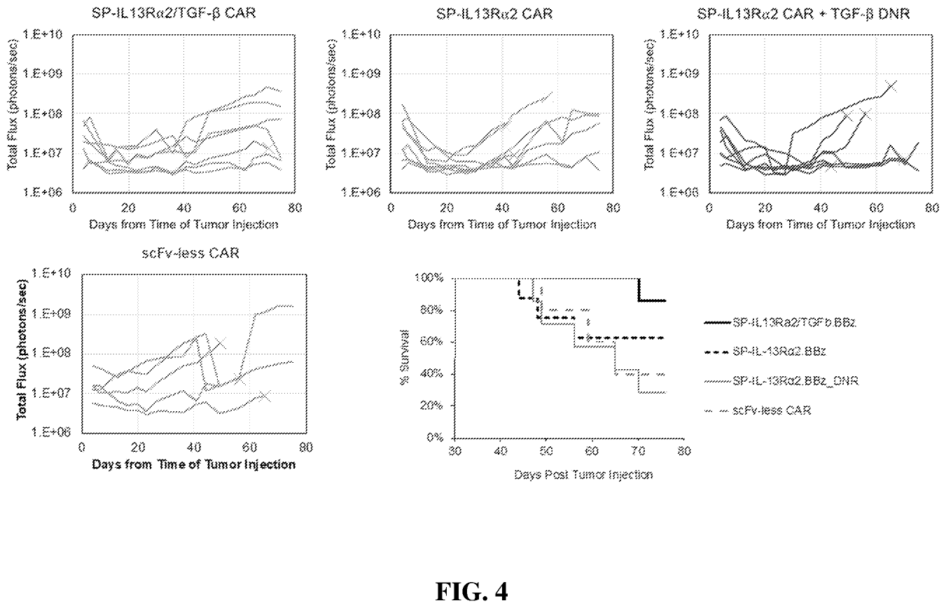

To assess anti-tumor function in vitro, CAR-T cells were labeled with CellTraceViolet (CTV) dye and co-cultured for 4 days with IL13Rα2+ PBT106 neurospheres at the indicated E:T ratios. Flow cytometry was performed to quantify viable tumor-cell count (FIG. 3A,B), viable T-cell count (FIG. 3C), viable CAR+ T-cell count (FIG. 3D), CTV dilution among all T cells (FIG. 3E), and CTV dilution among CAR+ T cells (FIG. 3F). FIG. 3B shows the same data as FIG. 3A, but with the scFv-less CAR condition removed to enable clear visualization. CARs bearing an N-terminal SP for the IL-13 mutein conferred more potent tumor-cell killing and greater T-cell proliferation (as assessed by CTV dilution) compared to CARs bearing the full IL-13 mutein N-terminus. Averages of triplicates are shown, with error bars representing ±1 standard deviation (FIG. 3).

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

The references recited in the application, to the extent that they provide exemplary procedural or other details supplementary to those set forth herein, are specifically incorporated herein by reference.

### REFERENCES

The following references and the publications referred to throughout the specification, to the extent that they provide exemplary procedural or other details supplementary to those set forth herein, are specifically incorporated herein by reference.
Brown C. E. et al. (2015). Bioactivity and safety of IL13Rα2-redirected chimeric antigen receptor CD8+ T cells in patients with recurrent glioblastoma. Clinical Cancer Research, 21: 4062-72.
Brown, C. E. et al. (2016). Regression of Glioblastoma after Chimeric Antigen Receptor T-Cell Therapy. New England Journal of Medicine, 375(26):2561-2596.
Chang, Z. L., Hou, A. J., and Chen, Y. Y. (2020). Engineering primary T cells with chimeric antigen receptors for rewired responses to soluble ligands. Nature Protocols, Epub ahead of print.
Chang, Z. L., Lorenzini, M. H., Zah, E., Tran, U., Chen, Y. Y. (2018). Rewiring T-cell responses to soluble factors with chimeric antigen receptors. Nature Chemical Biology, 14(3):317-324.
Debinski, W. and Thompson J. P. (1999). Retargeting interleukin 13 for radioimmunodetection and radioimmunotherapy of human high-grade gliomas. Clinical Cancer Research, 5:3143s-7s.
Hou, A. J., Chang, Z. L., Lorenzini, M. H., Zah, E., and Chen, Y. Y. (2018). TGF-β-responsive CAR-T cells promote anti-tumor immune function. Bioengineering and Translational Medicine, 3(2):75-86.
Kahlon, K. S. et al. (2004). Specific recognition and killing of glioblastoma multiforme by interleukin 13-zetakine redirected cytolytic T cells. Cancer Research, 64:9160-6.

What is claimed is:

1. A polypeptide comprising a multi-specific chimeric antigen receptor comprising an IL13 polypeptide with the amino acid sequence of SEQ ID NO:4 or 20, a glioblastoma antigen binding region, a peptide spacer, a transmembrane domain, and a cytoplasmic region comprising a co-stimulatory region and a primary intracellular signaling domain; wherein the glioblastoma antigen binding region comprises a GD2 or EGFRvIII binding region.

2. The polypeptide of claim 1, wherein the glioblastoma antigen binding region comprises a GD2 binding region.

3. The polypeptide of claim 2, wherein the GD2 binding region comprises an anti-GD2 scFv having a variable heavy (VH) and variable light (VL) region, wherein the VH region comprises the HCDR1, HCDR2; and HCDR3 from the VH of SEQ ID NO:46 and the VL region comprises LCDR1, LCDR2; and LCDR3 from the VL of SEQ ID NO:47.

4. The polypeptide of claim 2 or 3, wherein the GD2 binding region comprises an anti-GD2 scFv having a variable heavy (VH) and variable light (VL) region, wherein the VH region comprises SEQ ID NO:48 (HCDR1), SEQ ID NO:49 (HCDR2); and SEQ ID NO:50 (HCDR3) and the VL region comprises SEQ ID NO:51 (LCDR1), SEQ ID NO:52 (LCDR2); and SEQ ID NO:53 (LCDR3).

5. The polypeptide of any one of claims 2-4, wherein the GD2 binding region comprises a VH with an amino acid sequence having at least 80% sequence identity to SEQ ID NO:46 and/or a VL with an amino acid sequence having at least 80% sequence identity to SEQ ID NO:47.

6. The polypeptide of claim 5, wherein the GD2 binding region comprises a VH with the amino acid sequence of SEQ ID NO:46 and/or a VL with the amino acid sequence of SEQ ID NO:47.

7. The polypeptide of any one of claims 1-6, wherein the GD2 binding region comprises an anti-GD2 scFv having an amino acid sequence with at least 80% sequence identity to SEQ ID NO:26.

8. The polypeptide of claim 7, wherein the GD2 binding region comprises an anti-GD2 scFv having the amino acid sequence of SEQ ID NO:26.

9. The polypeptide of claim 1, wherein the glioblastoma antigen binding region comprises an EGFRvIII binding region.

10. The polypeptide of claim 9, wherein the EGFRvIII binding region comprises an anti-EGFRvIII scFv having a variable heavy (VH) and variable light (VL) region, wherein the VH region comprises the HCDR1, HCDR2; and HCDR3 from the VH of SEQ ID NO:38 and the VL region comprises LCDR1, LCDR2; and LCDR3 from the VL of SEQ ID NO:39.

11. The polypeptide of claim 9 or 10, wherein the EGFRvIII binding region comprises an anti-EGFRvIII scFv having a variable heavy (VH) and variable light (VL) region, wherein the VH region comprises SEQ ID NO:40 (HCDR1), SEQ ID NO:41 (HCDR2); and SEQ ID NO:42 (HCDR3) and the VL region comprises SEQ ID NO:43 (LCDR1), SEQ ID NO:44 (LCDR2); and SEQ ID NO:45 (LCDR3).

12. The polypeptide of any one of claims 9-11, wherein the EGFRvIII binding region comprises a VH with an amino acid sequence having at least 80% sequence identity to SEQ ID NO:38 and/or a VL with an amino acid sequence having at least 80% sequence identity to SEQ ID NO:39.

13. The polypeptide of claim 12, wherein the EGFRvIII binding region comprises a VH with the amino acid sequence of SEQ ID NO:38 and/or a VL with the amino acid sequence of SEQ ID NO:39.

14. The polypeptide of any one of claims 9-13, wherein the EGFRvIII binding region comprises an anti-EGFRvIII scFv having an amino acid sequence with at least 80% sequence identity to SEQ ID NO:27.

15. The polypeptide of claim 14, wherein the EGFRvIII binding region comprises an anti-EGFRvIII scFv having the amino acid sequence of SEQ ID NO:27.

16. The polypeptide of any one of claims 1-15, wherein the CAR comprises in order from amino-proximal end to carboxy-proximal end: an IL13 polypeptide, a glioblastoma antigen binding region, a peptide spacer, a transmembrane domain, and a cytoplasmic region comprising a co-stimulatory region and a primary intracellular signaling domain.

17. The polypeptide of any one of claims 1-16, wherein the polypeptide comprises a linker between the IL13 polypeptide and the glioblastoma antigen binding region.

18. The polypeptide of any one of claims 1-17, wherein the polypeptide comprises a tri-specific CAR comprising a TGF-β binding region.

19. The polypeptide of claim 18, wherein the CAR comprises in order from amino-proximal end to carboxy-proximal end: an IL13 polypeptide, a glioblastoma antigen binding region, a TGF-β binding region, a peptide spacer, a transmembrane domain, and a cytoplasmic region comprising a co-stimulatory region and a primary intracellular signaling domain.

20. The polypeptide of claim 18 or 19, wherein the polypeptide comprises a linker between the glioblastoma antigen binding region or the IL13 polypeptide and the TGF-β binding region.

21. The polypeptide of any one of claims 17-20, wherein the linker comprises glycine and serine amino acids.

22. The polypeptide of claim 21, wherein the linker comprises or consists of a polypeptide with the amino acid sequence of SEQ ID NO:10 or 28.

23. A polypeptide comprising a multi-specific chimeric antigen receptor (CAR) comprising an IL13 polypeptide with the amino acid sequence of SEQ ID NO:4 or 20, a TGF-β binding region, a peptide spacer, a transmembrane domain, and a cytoplasmic region comprising a co-stimulatory region and a primary intracellular signaling domain.

24. A polypeptide comprising a multi-specific chimeric antigen receptor (CAR) comprising a glioblastoma antigen binding region, a TGF-β binding region, a peptide spacer, a transmembrane domain, and a cytoplasmic region comprising a co-stimulatory region and a primary intracellular signaling domain; wherein the glioblastoma antigen binding region comprises an anti-GD2 scFv having a variable heavy (VH) and variable light (VL) region, wherein the VH region comprises the HCDR1, HCDR2; and HCDR3 from the VH of SEQ ID NO:46 and the VL region comprises LCDR1, LCDR2; and LCDR3 from the VL of SEQ ID NO:47.

25. The polypeptide of claim 24, wherein the anti-GD2 scFv comprises a variable heavy (VH) and variable light (VL) region, wherein the VH region comprises SEQ ID NO:48 (HCDR1), SEQ ID NO:49 (HCDR2); and SEQ ID NO:50 (HCDR3) and the VL region comprises SEQ ID NO:51 (LCDR1), SEQ ID NO:52 (LCDR2); and SEQ ID NO:53 (LCDR3).

26. The polypeptide of claim 24 or 25, wherein the GD2 binding region comprises a VH with an amino acid sequence having at least 80% sequence identity to SEQ ID NO:46 and/or a VL with an amino acid sequence having at least 80% sequence identity to SEQ ID NO:47.

27. The polypeptide of claim 26, wherein the GD2 binding region comprises a VH with the amino acid sequence of SEQ ID NO:46 and/or a VL with the amino acid sequence of SEQ ID NO:47.

28. The polypeptide of any one of claims 24-27, wherein the GD2 binding region comprises an anti-GD2 scFv having an amino acid sequence with at least 80% sequence identity to SEQ ID NO:26.

29. The polypeptide of claim 28, wherein the GD2 binding region comprises an anti-GD2 scFv having the amino acid sequence of SEQ ID NO:26.

30. A polypeptide comprising a multi-specific chimeric antigen receptor (CAR) comprising a glioblastoma antigen binding region, a TGF-β binding region, a peptide spacer, a transmembrane domain, and a cytoplasmic region comprising a co-stimulatory region and a primary intracellular signaling domain; wherein the glioblastoma antigen binding region comprises an EGFRvIII binding region.

31. The polypeptide of claim 30, wherein the EGFRvIII binding region comprises an anti-EGFRvIII scFv having a variable heavy (VH) and variable light (VL) region, wherein the VH region comprises the HCDR1, HCDR2; and HCDR3 from the VH of SEQ ID NO:38 and the VL region comprises LCDR1, LCDR2; and LCDR3 from the VL of SEQ ID NO:39.

32. The polypeptide of claim 30 or 31, wherein the EGFRvIII binding region comprises an anti-EGFRvIII scFv having a variable heavy (VH) and variable light (VL) region, wherein the VH region comprises SEQ ID NO:40 (HCDR1), SEQ ID NO:41 (HCDR2); and SEQ ID NO:42 (HCDR3) and the VL region comprises SEQ ID NO:43 (LCDR1), SEQ ID NO:44 (LCDR2); and SEQ ID NO:45 (LCDR3).

33. The polypeptide of any one of claims 30-32, wherein the EGFRvIII binding region comprises a VH with an amino acid sequence having at least 80% sequence identity to SEQ ID NO:38 and/or a VL with an amino acid sequence having at least 80% sequence identity to SEQ ID NO:39.

34. The polypeptide of claim 33, wherein the EGFRvIII binding region comprises a VH with the amino acid sequence of SEQ ID NO:38 and/or a VL with the amino acid sequence of SEQ ID NO:39.

35. The polypeptide of any one of claims 30-34, wherein the EGFRvIII binding region comprises an anti-EGFRvIII scFv having an amino acid sequence with at least 80% sequence identity to SEQ ID NO:27.

36. The polypeptide of claim 35, wherein the EGFRvIII binding region comprises an anti-EGFRvIII scFv having the amino acid sequence of SEQ ID NO:27.

37. The polypeptide of any one of claims 18-36, wherein the TGF-β binding region comprises a scFv having a variable heavy (VH) and variable light (VL) region, wherein the VH region comprises the HCDR1, HCDR2; and HCDR3 from the VH of SEQ ID NO:29 and the VL region comprises LCDR1, LCDR2; and LCDR3 from the VL of SEQ ID NO:30.

38. The polypeptide of any one of claims 18-37, wherein the TGF-β binding region comprises a scFv having a variable heavy (VH) and variable light (VL) region, wherein the VH region comprises SEQ ID NO:31 (HCDR1), SEQ ID NO:32 (HCDR2); and SEQ ID NO:33 (HCDR3) and the VL region comprises SEQ ID NO:34 (LCDR1), SEQ ID NO:35 (LCDR2); and SEQ ID NO:36 (LCDR3).

39. The polypeptide of any one of claims 18-38, wherein the scFv comprises a linker between the VH and VL regions.

40. The polypeptide of claim 39, wherein the linker comprises glycine and serine amino acid residues.

41. The polypeptide of claim 40, wherein the linker comprises or consists of the amino acid sequence of SEQ ID NO:10 or 28.

42. The polypeptide of any one of claims 23-41, wherein the TGF-β binding region comprises a VH with an amino acid sequence having at least 80% sequence identity to SEQ ID NO:29 and/or a VL with an amino acid sequence having at least 80% sequence identity to SEQ ID NO:30.

43. The polypeptide of claim 42, wherein the TGF-β binding region comprises a VH with the amino acid sequence of SEQ ID NO:29 and/or a VL with the amino acid sequence of SEQ ID NO:30.

44. The polypeptide of any one of claims 23-43, wherein the TGF-β binding region comprises an anti-TGF-β scFv having an amino acid sequence with at least 80% sequence identity to SEQ ID NO:11.

45. The polypeptide of claim 44, wherein the TGF-β binding region comprises an anti-TGF-β scFv having the amino acid sequence of SEQ ID NO:11.

46. The polypeptide of any one of claims 1-45, wherein the polypeptide further comprises a second chimeric antigen receptor comprising at least one antigen binding region, a second peptide spacer, a second transmembrane domain, and a second cytoplasmic region comprising a second co-stimulatory region and a second primary intracellular signaling domain.

47. The polypeptide of claim 46, wherein the second CAR is a mono-specific or multi-specific CAR.

48. The polypeptide of claim 46 or 47, wherein the second CAR comprises an antigen binding region to TGF-β.

49. The polypeptide of claim 48, wherein the TGF-β binding region comprises a scFv having a variable heavy (VH) and variable light (VL) region, wherein the VH region comprises the HCDR1, HCDR2; and HCDR3 from the VH of SEQ ID NO:29 and the VL region comprises LCDR1, LCDR2; and LCDR3 from the VL of SEQ ID NO:30.

50. The polypeptide of claim 48 or 49, wherein the TGF-β binding region comprises a scFv having a variable heavy (VH) and variable light (VL) region, wherein the VH region comprises SEQ ID NO:31 (HCDR1), SEQ ID NO:32 (HCDR2); and SEQ ID NO:33 (HCDR3) and the VL region comprises SEQ ID NO:34 (LCDR1), SEQ ID NO:35 (LCDR2); and SEQ ID NO:36 (LCDR3).

51. The polypeptide of claim 49 or 50, wherein the scFv comprises a linker between the VH and VL regions.

52. The polypeptide of claim 51, wherein the linker comprises glycine and serine amino acid residues.

53. The polypeptide of claim 52, wherein the linker comprises or consists of the amino acid sequence of SEQ ID NO:10.

54. The polypeptide of any one of claims 48-53, wherein the TGF-β binding region comprises a VH with an amino acid sequence having at least 80% sequence identity to SEQ ID NO:29 and/or a VL with an amino acid sequence having at least 80% sequence identity to SEQ ID NO:30.

55. The polypeptide of claim 54, wherein the TGF-β binding region comprises a VH with the amino acid sequence of SEQ ID NO:29 and/or a VL with the amino acid sequence of SEQ ID NO:30.

56. The polypeptide of any one of claims 48-55, wherein the TGF-β binding region comprises an anti-TGF-β scFv having an amino acid sequence with at least 80% sequence identity to SEQ ID NO:11.

57. The polypeptide of claim 56, wherein the TGF-β binding region comprises an anti-TGF-β scFv having the amino acid sequence of SEQ ID NO:11.

58. The polypeptide of any one of claims 46-56, wherein the first CAR and the second CAR are separated by one or more peptide cleavage site(s).

59. The polypeptide of claim 58, wherein the wherein the one or more cleavage sites comprise a 2A cleavage site.

60. The polypeptide of claim 59, wherein the 2A cleavage site comprises one or more of a P2A, F2A, E2A, or T2A cleavage site.

61. The polypeptide of claim 60, wherein the cleavage site comprise a T2A cleavage site with an amino acid sequence of SEQ ID NO:24 or with an amino acid sequence with at least 80% sequence identity to SEQ ID NO:24.

62. The polypeptide of any one of claims 1-61, wherein the peptide spacer is between the antigen binding domains and the transmembrane domain and/or the second peptide spacer is between the antigen binding domains and the second transmembrane domain of the second CAR.

63. The polypeptide of any one of claims 1-62, wherein the peptide spacer or second peptide spacer comprises an IgG4 hinge region.

64. The peptide spacer of claim 63, wherein the IgG4 hinge region comprises a polypeptide having an amino acid sequence with at least 80% sequence identity to SEQ ID NO:12 or 5.

65. The peptide spacer of claim 64, wherein the IgG4 hinge region comprises a polypeptide having the amino acid sequence of SEQ ID NO:12 or 5.

66. The polypeptide of any one of claims 1-65, wherein the peptide spacer or second peptide spacer comprises or further comprises an IgG4 CH2 and CH3 region.

67. The polypeptide of claim 66, wherein the IgG4 CH2 and CH3 region comprises a polypeptide having an amino acid sequence with at least 80% sequence identity to SEQ ID NO:37.

68. The peptide spacer of claim 64, wherein the IgG4 CH2 and CH3 region comprises a polypeptide having the amino acid sequence of SEQ ID NO:37.

69. The polypeptide of any one of claims 1-68, wherein the transmembrane domain or second transmembrane domain comprises the transmembrane domain from the CD28 protein.

70. The polypeptide of any one of claims 1-69, wherein the transmembrane domain or second transmembrane domain comprises a transmembrane domain having an amino acid sequence with at least 80% sequence identity to SEQ ID NO:6.

71. The polypeptide of any one of claims 1-70, wherein the transmembrane domain or second transmembrane domain comprises a transmembrane domain having the amino acid sequence of SEQ ID NO:6.

72. The polypeptide of any one of claims 1-71, wherein the co-stimulatory region or second co-stimulatory region comprises the co-stimulatory region from the 4-1BB protein or from the CD28 protein.

73. The polypeptide of any one of claims 1-72, wherein the co-stimulatory region or second co-stimulatory region comprises a co-stimulatory region having an amino acid sequence with at least 80% sequence identity to SEQ ID NO:7, 14, or 18.

74. The polypeptide of any one of claims 1-73, wherein the co-stimulatory region or second co-stimulatory region comprises a co-stimulatory region having the amino acid sequence of SEQ ID NO:7, 14, or 18.

75. The polypeptide of any one of claims 1-74, wherein the primary intracellular signaling domain or second primary intracellular signaling domain comprises an intracellular signaling domain from the CD3ζ protein.

76. The polypeptide of any one of claims 1-75, wherein the primary intracellular signaling domain or second primary intracellular signaling domain comprises an intracellular signaling domain having an amino acid sequence with at least 80% sequence identity to SEQ ID NO:8 or 15.

77. The polypeptide of any one of claims 1-76, wherein the primary intracellular signaling domain or second primary intracellular signaling domain comprises an intracellular signaling domain having the amino acid sequence of SEQ ID NO:8 or 15.

78. The polypeptide of any one of claims 1-77, wherein the polypeptide further comprises one or more molecular tag(s).

79. The polypeptide of claim 78, wherein the one or more molecular tags comprise FLAG and/or HA tag.

80. The polypeptide of any one of claims 1-79, wherein the CAR and/or second CAR comprises a torsional linker between the transmembrane domain and the cytoplasmic region.

81. The polypeptide of claim 80, wherein the torsional linker comprises 1, 2, 3, 4, 5, 6, 7, 8, 9, or 10 amino acid residues.

82. The polypeptide of claim 81, wherein the amino acid residues comprise or consist of alanine residues.

83. The polypeptide of claim 82, wherein the torsional linker consists of 2 or 4 alanine residues.

84. The polypeptide of any one of claims 1-83, wherein the polypeptide comprises one of SEQ ID NOS:136-147 or an amino acid sequence having at least 80% sequence identity to one of SEQ ID NOS:136-147.

85. The polypeptide of any one of claims 1-84, wherein the polypeptide further comprises one or more signal sequence(s).

86. The polypeptide of claim 85, wherein the signal sequence(s) comprise an amino acid sequence with at least 80% sequence identity to SEQ ID NO:2.

87. The polypeptide of claim 86, wherein the signal sequence(s) comprise the amino acid sequence of SEQ ID NO:2.

88. An isolated nucleic acid encoding the polypeptide of any one of claims 1-87.

89. The nucleic acid of claim 88, wherein the nucleic acid is an expression construct.

90. The nucleic acid of claim 89, wherein the expression construct is a viral vector.

91. The nucleic acid of claim 90, wherein the viral vector comprises a retroviral vector a vector derived from a retrovirus.

92. The nucleic acid of claim 91, wherein the viral vector is a lentiviral vector or a vector derived from a lentivirus.

93. A lentivirus vector comprising a sequence encoding the polypeptide of any one of claims 1-87.

94. A cell comprising the nucleic acid of any of claims 88-93.

95. The cell of claim 94, wherein the viral vector has integrated into the cell's genome.

96. The cell of claim 94 or 95, wherein the cell further comprises a second nucleic acid encoding a second CAR.

97. The cell of claim 96, wherein the second CAR is a mono-specific or multi-specific CAR.

98. The cell of claim 96 or 97, wherein the second CAR comprises an antigen binding region to TGF-β.

99. The cell of claim 98, wherein the TGF-β binding region comprises a scFv having a variable heavy (VH) and variable light (VL) region, wherein the VH region comprises the HCDR1, HCDR2; and HCDR3 from the VH of SEQ ID NO:29 and the VL region comprises LCDR1, LCDR2; and LCDR3 from the VL of SEQ ID NO:30.

100. The cell of claim 98 or 99, wherein the TGF-β binding region comprises a scFv having a variable heavy (VH) and variable light (VL) region, wherein the VH region comprises SEQ ID NO:31 (HCDR1), SEQ ID NO:32 (HCDR2); and SEQ ID NO:33 (HCDR3) and the VL region comprises SEQ ID NO:34 (LCDR1), SEQ ID NO:35 (LCDR2); and SEQ ID NO:36 (LCDR3).

101. The cell of claim 99 or 100, wherein the scFv comprises a linker between the VH and VL regions.

102. The cell of claim 101, wherein the linker comprises glycine and serine amino acid residues.

103. The cell of claim 102, wherein the linker comprises or consists of the amino acid sequence of SEQ ID NO:10 or 28.

104. The cell of any one of claims 98-103, wherein the TGF-β binding region comprises a VH with an amino acid sequence having at least 80% sequence identity to SEQ ID NO:29 and/or a VL with an amino acid sequence having at least 80% sequence identity to SEQ ID NO:30.

105. The cell of any one of claims 98-104, wherein the TGF-β binding region comprises a VH with the amino acid sequence of SEQ ID NO:29 and/or a VL with the amino acid sequence of SEQ ID NO:30.

106. The cell of any one of claims 98-105, wherein the TGF-β binding region comprises an anti-TGF-β scFv having an amino acid sequence with at least 80% sequence identity to SEQ ID NO:11.

107. The cell of claim 106, wherein the TGF-β binding region comprises an anti-TGF-β scFv having the amino acid sequence of SEQ ID NO:11.

108. The cell of any one of claims 96-107, wherein the second CAR comprises an antigen binding region to EGFRvIII.

109. The cell of any one of claims 96-108, wherein the second CAR comprises an antigen binding region to GD2.

110. The cell of any one of claims 94-99, wherein the cell is ex vivo.

111. A cell expressing the polypeptide of any of claims 1-87.

112. The cell of claim 111, wherein the cell further comprises a second polypeptide comprising a second CAR.

113. The cell of claim 112, wherein the second CAR is a mono-specific or multi-specific CAR.

114. The cell of claim 112 or 113, wherein the second CAR comprises an antigen binding region to TGF-β.

115. The cell of claim 114, wherein the TGF-β binding region comprises a scFv having a variable heavy (VH) and variable light (VL) region, wherein the VH region comprises the HCDR1, HCDR2; and HCDR3 from the VH of SEQ ID NO:29 and the VL region comprises LCDR1, LCDR2; and LCDR3 from the VL of SEQ ID NO:30.

116. The cell of claim 114 or 115, wherein the TGF-β binding region comprises a scFv having a variable heavy (VH) and variable light (VL) region, wherein the VH region comprises SEQ ID NO:31 (HCDR1), SEQ ID NO:32 (HCDR2); and SEQ ID NO:33 (HCDR3) and the VL region comprises SEQ ID NO:34 (LCDR1), SEQ ID NO:35 (LCDR2); and SEQ ID NO:36 (LCDR3).

117. The cell of claim 115 or 116, wherein the scFv comprises a linker between the VH and VL regions.

118. The cell of claim 117, wherein the linker comprises glycine and serine amino acid residues.

119. The cell of claim 118, wherein the linker comprises or consists of the amino acid sequence of SEQ ID NO:10 or 28.

120. The cell of any one of claims 114-119, wherein the TGF-β binding region comprises a VH with an amino acid sequence having at least 80% sequence identity to SEQ ID NO:29 and/or a VL with an amino acid sequence having at least 80% sequence identity to SEQ ID NO:30.

121. The cell of any one of claims 114-120, wherein the TGF-β binding region comprises a VH with the amino acid sequence of SEQ ID NO:29 and/or a VL with the amino acid sequence of SEQ ID NO:30.

122. The cell of any one of claims 114-121, wherein the TGF-β binding region comprises an anti-TGF-β scFv having an amino acid sequence with at least 80% sequence identity to SEQ ID NO:11.

123. The cell of claim 122, wherein the TGF-β binding region comprises an anti-TGF-β scFv having the amino acid sequence of SEQ ID NO:11.

124. The cell of any one of claims 112-123, wherein the second CAR comprises an antigen binding region to EGFRvIII.

125. The cell of any one of claims 112-124, wherein the second CAR comprises an antigen binding region to GD2.

126. The cell of any of claims 94-125, wherein the cell is a T cell, a natural killer (NK) cell, a natural killer T cell (NKT), an invariant natural killer T cell (iNKT), stem cell, lymphoid progenitor cell, peripheral blood mononuclear cell (PBMC), bone marrow cell, fetal liver cell, embryonic stem cell, hematopoietic stem or progenitor cell (HSPC), cord blood cell, or induced pluripotent stem cell (iPS cell).

127. The cell of claim 126, wherein the cell is a T cell or an NK cell.

128. The cell of claim 127, wherein the T cell comprises a naïve memory T cell.

129. The cell of claim 128, wherein the naïve memory T cell comprises a CD4+ or CD8+ T cell.

130. A population of cell comprising any of the cells of claims 123-129.

131. The population of cells of claim 130, wherein the population comprises 10³-10⁸ cells.

132. A composition comprising the population of cells of claim 130 or 131, wherein the composition is a pharmaceutically acceptable formulation.

133. A method of making a cell that expresses a polypeptide comprising introducing into a cell the nucleic acid of any of claims 88-93.

134. The method of claim 133, wherein the cell is infected with a virus encoding the polypeptide.

135. The method of claim 134, wherein the virus comprises lentivirus or a lentiviral-derived virus or vector.

136. The method of any one of claims 133-135, wherein the cell is a T cell, a natural killer (NK) cell, a natural killer T cell (NKT), an invariant natural killer T cell (iNKT), stem cell, lymphoid progenitor cell, peripheral blood mononuclear cell (PBMC), bone marrow cell, fetal liver cell, embryonic stem cell, cord blood cell, induced pluripotent stem cell (iPS cell).

137. The method of claim 136, wherein the cell is a T cell or an NK cell.

138. The method of claim 137, wherein the T cell comprises a naïve memory T cell.

139. The method of claim 138, wherein the naïve memory T cell comprises a CD4+ or CD8+ T cell.

140. The method of any one of claims 133-139, wherein the cell is not yet a T cell or NK cell, the method further comprising culturing the cell under conditions that promote the differentiation of the cell into a T cell or an NK cell.

141. The method of any of claims 133-140, further comprising culturing the cell under conditions to expand the cell before and or after introducing the nucleic acid into the cell.

142. The method of claim 141, wherein the cell is cultured with serum-free medium.

143. A method of treating a subject with glioblastoma comprising administering to the subject an effective amount of the composition of claim 132.

144. The method of claim 143, wherein the method further comprises administering an additional therapy to the subject.

145. The method of claim 144, wherein the additional therapy comprises an immunotherapy.

146. The method of any one of claims 143-145, wherein the composition is administered intraventricularly, intracerebroventricularly, intratumorally, intravenously, or into a tumor resection cavity.

147. A method for stimulating an immune response or for treating cancer in a subject, the method comprising administering to the subject an effective amount of the composition of claim 132.

148. The method of claim 147, wherein stimulating an immune response comprises increasing expression and/or secretion of immune stimulating cytokines and/or molecules.

149. The method of claim 147 or 148, wherein the immune stimulating cytokines and/or molecules are one or more of TNF-α, IFN-β, IFN-γ, IL-1, IL-2, IL-4, IL-6, IL-8, IL-10, IL-12, IL-18 and granulocyte-macrophage colony stimulating factor.

150. The method of any one of claims 147-149, wherein stimulating an immune response comprises increasing proliferation of immune cells.

151. The method of claim 150, wherein the immune cells are T cells.

152. The method of any one of claims 147-151, wherein the cell is in vivo in a subject in need of immune stimulation.

153. The method of claim 152, wherein the subject is one that produces endogenous TGF-β.

154. The method of any one of claims 147-153, wherein the cancer comprises glioblastoma.

155. The method of any one of claims 147-154 wherein the wherein the subject is a human subject.

156. The method of any one of claims 147-155, wherein the method further comprises administering TGF-β to the subject.

157. A method for expanding therapeutic T cells in vitro, the method comprising contacting the in vitro T cell of any one of claims 127-129 with a composition comprising TGF-β.

158. The method of claim 157, wherein the composition comprises 1-50 ng/mL of TGF-β.

159. The method of claim 157 or 158, wherein the composition further comprises IL-2.

160. The method of claim 159, wherein the composition comprises 20-400 U/mL of IL-2 and/or 0.1-10 ng/ml IL-15.

161. The method of any one of claims 157-160, wherein the method further comprises contacting the cells with feeder cells.

162. The method of claim 161, wherein the feeder cells are irradiated.

163. The method of any one of claims 157-162, wherein the method excludes contact of the T cells with feeder cells.
